(19) United States
(12) Patent Application Publication
(10) Pub. No.: US 2008/0125418 A1
(43) Pub. Date: May 29, 2008

(54) Benzimidazole derivatives and their use as kdr kinase protein inhibitors

(75) Inventors: Didier Babin, Montigny (FR); Alain Le Brun, Vigneux (FR); Laurence Gauzy-Lazo, Paris (FR); Herve Bouchard, Thiais (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 11/943,008

(22) Filed: Nov. 20, 2007

Related U.S. Application Data
(63) Continuation of application No. 10/828,012, filed on Apr. 20, 2004, now abandoned.
Application No. 10/828,012 is a continuation of application No. PCT/FR02/03647, filed on Oct. 24, 2002.

(30) Foreign Application Priority Data
Oct. 26, 2001 (FR) 0113867

(51) Int. Cl. A61K 31/541; A61K 31/5377; A61K 31/496; A61K 31/454; A61K 31/4196; A61K 31/4184; C07D 417/02; C07D 413/02; C07D 403/02

(52) U.S. Cl. 514/227.8; 514/383; 514/394; 514/322; 514/233.2; 514/254.06; 544/60; 544/139; 544/368; 548/266.4; 548/305.1; 546/199

(57) ABSTRACT

The invention discloses and claims benzimidazole compounds of formula (I):

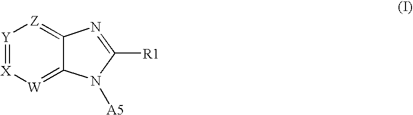

wherein X is C—R2; Y is C—R2 or C—R3; W and Z are each C—R3; R1 is an optionally substituted aryl, heteroaryl or a saturated 5- or 6-membered monocyclic heterocyclic radical or a bicyclic heterocyclic radical; and A5 is H or alkyl; or a stereoisomer, a racemate, an enantiomer or a diastereoisomer of said compound of formula (I) or a pharmaceutically acceptable salt thereof; the use of compounds of formula (I) for the treatment of a disorder of proliferation of blood vessels, uncontrolled angiogenesis, a fibrotic disorder, a disorder of proliferation of mesangial cells, a metabolic disorder, allergy, asthma, thrombosis, a disease of the nervous system, retinopathy, psoriasis, rheumatoid arthritis, diabetes, muscle degeneration, solid tumors and cancers, pharmaceutical compositions comprising a compound of formula (I) and one or more pharmaceutically acceptable adjuvants or diluents and pharmaceutical compositions comprising a compound of formula (I) and one or more. antimitotic agents.

### BACKGROUND OF THE INVENTION

1. Field of the Invention

The present invention relates to novel benzimidazole derivatives, to a process for preparing them, to the novel intermediates obtained, to their use as medicinal products, to pharmaceutical compositions containing them and to the novel use of such benzimidazole derivatives.

2. Description of the Art

One subject of the invention is thus novel benzimidazole derivatives with inhibitory effects towards kinase proteins.

The benzimidazoles of the present invention may thus especially be used for preventing or treating diseases that may be modulated by the inhibition of kinase proteins.

Such kinase proteins belong especially to the following group: EGFR, Fak, FLK-1, FGFR1, FGFR2, FGFR3, FGFR4, FGFR5, flt-1, IGF-1R¹, KDR, PDGFR, tie2 and VEGFR.

Mention is made more particularly of the kinase protein KDR.

Mention is also made particularly of the kinase protein Tie-2.

Kinase proteins are a family of enzymes that catalyse the phosphorylation of hydroxyl groups of specific residues of proteins, such as tyrosine, serine or threonine residues. Such phosphorylations may greatly modify the function of the proteins; thus, kinase proteins play an important role in regulating a wide variety of cell processes including, especially, metabolism, cell proliferation, cell differentiation or cell survival. Among the various cellular functions in which the activity of a kinase protein is involved, certain processes represent attractive targets for treating certain diseases. As an example, mention may be made especially of angiogenesis and the control of the cell cycle, in which kinase proteins can play an essential role. These processes are essential for the growth of solid tumours and also for other diseases.

Angiogenesis is the process in which new vessels are formed from already existing vessels. Should the need arise, the vascular system has the potential to generate a network of new vessels so as to maintain the correct functioning of the tissues and organs.

Angiogenesis is a complex multi-step process involving activation, migration, proliferation and survival of endothelial cells.

In adults, angiogenesis is fairly limited, appearing mainly only in the processes of repair after an injury or of vascularization of the endometrium (Merenmies et al., Cell Growth & Differentiation, 8, 3-10, 1997). However, uncontrolled angiogenesis is found in certain pathologies such as retinopathy, psoriasis, rheumatoid arthritis, diabetes, muscle degeneration or cancer (solid tumours) (Folkman, Nature Med., 1, 27-31, 1995). The kinase proteins whose involvement it has been possible to demonstrate in the angiogenesis process include three members of the family of growth factor receptor tyrosine kinases: VEGF-R2 (vascular endothelial growth factor receptor 2, also known as KDR, kinase insert domain receptor, or FLK-1), FGF-R (fibroblast growth factor receptor) and TEK (also known as Tie-2).

In conjunction with other systems, the Vascular Endothelial Growth Factor receptors (VEGFRs) transmit signals involved in the migration, proliferation and survival of endothelial cells. The family VEGFR includes VEGFR-1 (Flt-1), VEGFR-2 (KDR) and VEGFR3 (Flt4).

The receptor VEGF-R2, which is expressed only in the endothelial cells, binds to the angiogenic growth factor VEGF, and thus serves as a transduction signal mediator via the activation of its intracellular kinase domain. Thus, the direct inhibition of the kinase activity of VEGF-R2 makes it possible to reduce the phenomenon of angiogenesis in the presence of exogenous VEGF (Strawn et al., Cancer Research, 56, 3540-3545, 1996), this process being demonstrated especially with the aid of VEGF-R2 mutants (Millauer et al., Cancer Research, 56, 1615-1620, 1996). The VEGF-R2 receptor appears to have no other function in adults than that associated with the angiogenic activity of VEGF. Thus, a selective inhibitor of the kinase activity of VEGF-R2 should show only little toxicity.

In addition to this central role in the dynamic angiogenic process, recent results suggest that the expression of VEGF contributes towards the survival of tumoral cells after chemotherapy and radiotherapy, underlining the potential synergism of KDR inhibitors with other agents (Lee C. G., Heijn M. et al., (2000), Cancer Research, 60 (19), 5565-70).

The KDR inhibitors thus especially constitute anti-angiogenic agents.

Angiogenesis inhibitors might thus be used as a first line treatment against the emergence or regrowth of malignant tumours.

The inhibition or regulation of VEGFR-2 (KDR) thus provides a powerful new mechanism of action for the treatment of a large number of solid tumours.

The present invention thus relates particularly to novel inhibitors of the VEGFR-2 (KDR) receptor that may be used especially for anti-angiogenic treatment in oncology.

The products of the present invention as KDR inhibitors may be used especially for the treatment or prevention of diseases chosen from the following group: cancers, especially breast, colon, lung and prostate cancer, atherosclerosis, degenerative muscle diseases, obesity, congestive heart failure, Parkinson's, depression, schizophrenia, stroke, head trauma, spinal cord injury, Alzheimer's, neuropathic pain syndrome, amyotrophic lateral sclerosis, cachexia, osteoporosis and fibrotic diseases of the viscera.

### SUMMARY OF THE INVENTION

One subject of the present invention is thus the products of formula (I):

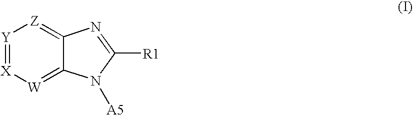

in which:
X represents C—R2 and W, Y and Z, which may be identical or different, represent CH or CR3;
R1 represents aryl or heteroaryl chosen from pyrazolyl, triazolyl, imidazolyl, indolyl, indazolyl, thienopyrazolyl, tetrahydroindazolyl, tetrahydrocyclopentapyrazolyl, dihydrofuropyrazolyl, oxodihydropyridazinyl, tetrahydropyrrolopyrazolyl, oxotetrahydropyrrolopyrazolyl, tetrahydropyranopyrazolyl, tetrahydropyridinopyrazolyl, and oxodihydropyridinopyrazolyl radicals, all these radicals being optionally substituted with one or more radicals X1, X2 or X3 chosen from H, halogen, haloalkyl, OH, R4, NO₂, CN, S(O)ₙR4, OR4, NY1Y2, COR4, —C(═O)NY1Y2, —C(═O)OR4, —C(═O)OH, —N(R6)C(═O)R4, —N(R6)SO₂R4, —N(R6)C(═O)NY1Y2, —N(R6)C(═O)OR4, —S(O)ₙOR4, —S(O)ₙNY1Y2, —OC(═O)NY1Y2, —OS(O)ₙR4, —OC(═O)R4 and optionally substituted thienyl,
R2 and R3 are such that:
either R2 and R3, which may be identical or different, represent H, R4, halogen, haloalkyl, OH, NO₂, CN, OR4, COR4, S(O)ₙR4, —C(═O)NY1Y2, —C(═O)OR4, —C(═O)OH, —NY1Y2, —N(R6)C(═O)R4, —N(R6)SO₂R4, —N(R6)C(═O)NY1Y2, —N(R6)C(═O)OR4, —S(O)ₙOR4, —S(O)ₙNY1Y2, —OC(═O)NY1Y2 and —OC(═O)R4
or R2 represents H, R4, halogen, haloalkyl, OH, NO₂, CN, OR4, COR4, S(O)ₙR4, —C(═O)NY1Y2, —C(═O)OR4, —C(═O)OH, —NY1Y2, —N(R6)C(═O)R4, —N(R6)SO₂R4, —N(R6)C(═O)NY1Y2, —N(R6)C(═O)OR4, —S(O)ₙOR4, —S(O)ₙNY1Y2, —OC(═O)NY1Y2 and —OC(═O)R4
and R3 represents alkyl, haloalkyl, halogen and OR6
or R2 and R3 together form a 5- to 6-membered carbon-based ring containing one or more hetero atoms, which may be identical or different, chosen from O, N and S,
R4 represents alkyl, alkenyl, alkynyl, cycloalkyl, aryl, heteroaryl, cycloalkylalkyl, heterocycloalkyl, heteroarylalkyl and arylalkyl, all these radicals being optionally substituted with one or more radicals chosen from aryl (optionally substituted), halogen, alkyl, hydroxyalkyl, OH, ORS, C(═O)NY3Y4, NY3Y4, alk-NY3Y4 and C(═O)OR6,
R5 represents alkyl, alkenyl, cycloalkyl, heterocycloalkyl, aryl, heteroaryl, arylalkyl, cycloalkylalkyl, heteroarylalkyl and heterocycloalkylalkyl.

Y1 and Y2 are such that: either Y1 and Y2, which may be identical or different, represent H and optionally substituted alkyl, alkenyl, cycloalkyl, heterocycloalkyl, heterocycloalkylalkyl, aryl, arylalkyl, heteroaryl and heteroarylalkyl,

or Y1 and Y2 form, together with the nitrogen atom to which they are attached, a cyclic amino radical,
Y3 and Y4 are such that: either Y3 and Y4, which may be identical or different, represent hydrogen, alkenyl, alkyl, aryl, arylalkyl, cycloalkyl, heteroaryl or heteroarylalkyl or Y3 and Y4 form, together with the nitrogen atom to which they are attached, an optionally substituted cyclic amino radical,
A5 represents H or alkyl,
R6 is chosen from the values of R5,
all the alkyl (or alk, which represents alkyl), alkenyl, cycloalkyl, heterocycloalkyl, aryl, arylalkyl, heteroaryl and heteroarylalkyl radicals present in the above radicals furthermore being optionally substituted with one or more radicals chosen from halogen atoms and hydroxyl, cyano, alkyl, alkoxy, acylamino (NH—COalk), —C(═O)OR6, acyl —C(═O)R6, hydroxyalkyl, carboxyalkyl, S(O)n-alk, S(O)n-NH₂, S(O)n-NH(alk), S(O)n-N(alk)2, CF3, OCF3, NO2, arylalkoxy, aryl, heteroaryl, aryloxy, aryloxyalkyl, —C(═O)—NY3Y4 and NY3Y4 radicals,
the latter radicals containing alkyl, aryl and heteroaryl being themselves optionally substituted with one or more radicals chosen from halogen atoms and alkyl radicals, free, salified (ionized or salt form) or esterified carboxyl radicals and acylamino radicals NH—C(O)R5,
the phenyl radicals furthermore being optionally substituted with a dioxole (methylenedioxy) radical,
n represents an integer from 0 to 2,
it being understood that when R1 represents an indazolyl radical
to give the products of formula (I) below:

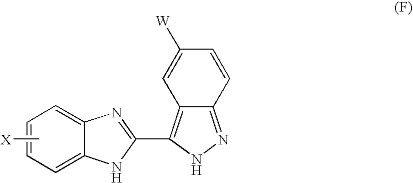

with X representing H, R2 or R3 as defined above, then W necessarily represents H or unsubstituted alkyl,
the said products of formula (I) being in any possible racemic, enantiomeric or diastereoisomeric isomer form, and also the addition salts with mineral and organic acids or with mineral bases.

One subject of the present invention is thus the products of formula (I) as defined above corresponding to the formula (Ia):

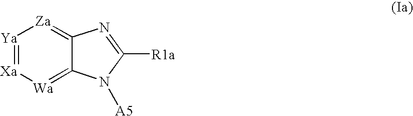

in which:
Xa represents C—R2a and Wa, Ya and Za, which may be identical or different, represent CH or CR3a;
R1a represents aryl or heteroaryl chosen from pyrazolyl, triazolyl and indazolyl radicals, all these radicals being optionally substituted with one or more radicals X1a, X2a or X3a chosen from H, halogen, OH, R4a, OR4a, NY1aY2a, S(O)nR4a, —C(═O)NY1aY2a, —C(═O)OR4a, —N(R6a)C(═O)R4a, —N(R6a)SO2R4a, —N(R6a)C(═O)NY1aY2a, —N(R6a)C(═O)OR4a, —OC(═O)NY1aY2a, —OC(═O)R4a, —OS(O)nR4a and thienyl optionally substituted with an alkyl radical, R2a and R3a are such that:
either R2a and R3a, which may be identical or different, represent H, R4a, halogen, OH, OR4a, C(═O)NY1aY2a, —C(═O)OR4a and —C(═O)OH, and R3a represents alkyl, halogen and OR6a,
or R2a represents H, R4a, halogen, OH, OR4a, C(═O)NY1aY2a, —C(═O)OR4a and —C(═O)OH, and R3a represents alkyl, halogen and OR6a,
or R2a and R3a together form an —O—CH2—O— or —O—CH2—CH2-O— ring,
R4a represents alkyl, alkenyl, cycloalkyl, aryl, heteroaryl, cycloalkylalkyl, heterocycloalkyl, heteroarylalkyl and arylalkyl, all these radicals being optionally substituted with one or more radicals chosen from aryl (optionally substituted), halogen, alkyl, hydroxyalkyl, OH, OR5a, C(═O)NY3aY4a, NY3aY4a, alk-NY3aY4a and C(═O)OR6a,
R5a represents alkyl, alkenyl, cycloalkyl, heterocycloalkyl, aryl, heteroaryl, arylalkyl, cycloalkylalkyl, heteroarylalkyl and heterocycloalkylalkyl, all these radicals being optionally substituted,
Y1a and Y2a are such that: either Y1a and Y2a, which may be identical or different, represent H, alkyl, alkoxyalkyl, aryloxyalkyl, arylalkyl, heteroarylalkyl, heterocycloalkylalkyl, cycloalkyl, aryl and heteroaryl, all these radicals being optionally substituted, or Y1a and Y2a form, together with the nitrogen atom to which they are attached, an optionally substituted cyclic amino radical,
Y3a and Y4a are such that: either Y3a and Y4a, which may be identical or different, represent hydrogen, alkyl, aryl, arylalkyl, cycloalkyl, heteroaryl or heteroarylalkyl, or Y3a and Y4a form, together with the nitrogen atom to which they are attached, a cyclic amino radical,
A5 represents H or alkyl,
all the alkyl, alkenyl, cycloalkyl, heterocycloalkyl, aryl, arylalkyl, heteroaryl and heteroarylalkyl radicals present in the above radicals furthermore being optionally substituted with one or more radicals chosen from halogen atoms and hydroxyl, cyano, alkyl, alkoxy, acylamino (NH—C(O)R6a), —C(═O)OR6a, acyl —C(═O)R6a, hydroxyalkyl, carboxyalkyl, S(O)n-alk, S(O)n-NH₂, S(O)n-NH(alk), S(O)n—N(alk)2, CF3, OCF3, NO2, arylalkoxy, aryl, heteroaryl, aryloxy, aryloxyalkyl, —C(═O)—NY3aY4a and NY3aY4a radicals,
the latter radicals containing alkyl, aryl and heteroaryl themselves being optionally substituted with one or more radicals chosen from halogen atoms and alkyl radicals, alkoxy radicals, free, salified or esterified carboxyl radicals and acylamino radicals NH—C(O)R6a,
the phenyl radicals furthermore being optionally substituted with a dioxole radical,
R6a is chosen from the values of R5a,
n represents an integer from 0 to 2,
the said products of formula (Ia) being in any possible racemic, enantiomeric or diastereoisomeric isomer form, and also the addition salts with mineral and organic acids or with mineral bases.

One subject of the present invention is thus the products of formula (I):

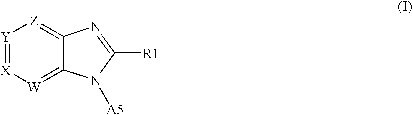

in which:
X represents C—R2 and W, Y and Z, which may be identical or different, represent CH or CR3;
R1 represents aryl or heteroaryl chosen from pyrazolyl, triazolyl, imidazolyl, indolyl, indazolyl, thienopyrazolyl, tetrahydroindazolyl, tetrahydrocyclopentapyrazolyl, dihydrofuropyrazolyl, oxodihydropyridazinyl, tetrahydropyrrolopyrazolyl, oxotetrahydropyrrolopyrazolyl, tetrahydropyranopyrazolyl, tetrahydropyridinopyrazolyl, and oxodihydropyridinopyrazolyl radicals, all these radicals optionally being substituted with one or more radicals X1, X2 or X3 chosen from H, halogen, haloalkyl, OH, R4, NO2, CN, S(O)nR4, OR4, NY1Y2, COR4, —C(═O)NY1Y2, —C(═O)OR4, —C(═O)OH, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2, —OS(O)nR4, —OC(═O)R4 and optionally substituted thienyl,
R2 and R3 are such that:
either R2 and R3, which may be identical or different, represent H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2 and —OC(═O)R4
or R2 represents H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2 and —OC(═O)R4
and R3 represents alkyl, haloalkyl, halogen and OR6
or R2 and R3 together form a 5- to 6-membered carbon-based ring containing one or more hetero atoms, which may be identical or different, chosen from O, N and S,
R4 represents alkyl, alkenyl, cycloalkyl, aryl, heteroaryl, cycloalkylalkyl, heterocycloalkyl, heteroarylalkyl and arylalkyl, all these radicals being optionally substituted with one or more radicals chosen from aryl, OH, OR5, C(═O)NY3Y4, NY3Y4 and C(═O)OR6,
R5 represents alkyl, alkenyl, cycloalkyl, heterocycloalkyl, aryl, heteroaryl, arylalkyl, cycloalkylalkyl, heteroarylalkyl and heterocycloalkylalkyl.
R6 represents H and C1-C4 alkyl,
n represents an integer from 0 to 2
Y1 and Y2 are such that: either Y1 and Y2, which may be identical or different, represent H, alkyl, alkenyl, cycloalkyl, aryl, arylalkyl, heteroaryl or heteroarylalkyl, all these radicals being optionally substituted with one or more radicals chosen from hydroxyl, —C(═O)—NY3Y4, —C(═O)OR6 and NY3Y4,
or Y1 and Y2 form, together with the nitrogen atom to which they are attached, a cyclic amino radical,
Y3 and Y4 are such that: either Y3 and Y4, which may be identical or different, represent hydrogen, alkenyl, alkyl, aryl, arylalkyl, cycloalkyl, heteroaryl or heteroarylalkyl or Y3 and Y4 form, together with the nitrogen atom to which they are attached, a cyclic amino radical, A5 represents H or alkyl,
it being understood that when R1 represents an indazolyl radical
to give the products of formula (I) below:

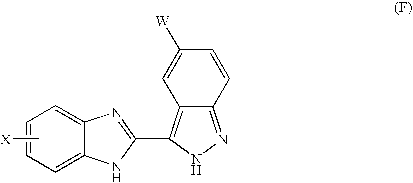

with X representing H, R2 or R3 as defined above, then W necessarily represents H or unsubstituted alkyl,
the said products of formula (I) being in any possible racemic, enantiomeric or diastereoisomeric isomer form, and also the addition salts with mineral and organic acids or with mineral bases.

It is obvious that, according to the ring represented by R1 and its number of members, R1 can comprise one, two or three substituents represented by X1, X2 and X3.

In the products of formula (I) and in the text hereinbelow:
the term “alkyl radical” denotes the linear and, where required, branched methyl, ethyl, propyl, isopropyl, butyl, isobutyl, sec-butyl, tert-butyl, pentyl, isopentyl, hexyl and isohexyl and also heptyl, octyl, nonyl and decyl radicals, and also the linear or branched positional isomers thereof,
the term “hydroxyalkyl radical” denotes the alkyl radicals indicated above substituted with at least one hydroxyl radical,
the term “alkenyl” denotes linear or branched radicals containing not more than 10 carbon atoms and containing one or more double bonds: mention may be made especially of vinyl, 1-propenyl, allyl, butenyl and 3-methyl-2-butenyl radicals, but also, for example, septa-, octa-, nona- or deca-dienyl radicals, such as, for example, the octa-2,6-dienyl radical,
the term “alkynyl” denotes linear or branched radicals containing not more than 10 carbon atoms: mention may be made especially of the alkyl radicals described above containing 2 to 10 carbon atoms and containing one or two triple bonds,
the term “alkylthio” denotes linear or branched radicals containing not more than 6 carbon atoms such as, especially, methylthio, ethylthio, propylthio, isopropylthio, butylthio, isobutylthio, sec-butylthio, tert-butylthio, pentylthio, isopentylthio, hexylthio or isohexylthio radicals, and also the linear or branched positional isomers thereof: among these alkylthio radicals, among those mentioned above, the ones preferably chosen are those containing not more than 4 carbon atoms,
the term “alkoxy radical” denotes the linear and, where required, branched methoxy, ethoxy, propoxy, isopropoxy, linear, secondary or tertiary butoxy, pentoxy or hexoxy radicals containing not more than 10 carbon atoms, and also the linear or branched positional isomers thereof,
the term “alkenyloxy radical” denotes the linear and branched —O-alkenyl radicals with alkenyl as defined above,
the terms “NH(alk)” and “N(alk) (alk)” denote an amino radical substituted, respectively, with one or two alkyl radicals, such alkyl radicals being linear or branched and chosen from alkyl radicals as defined above, preferably containing not more than 4 carbon atoms,
the term “acyl” denotes a radical R—C(O)— in which R represents a radical chosen from a hydrogen atom, linear or branched alkyl radicals containing not more than 6 carbon atoms; optionally substituted amino as defined above, aryl, heteroaryl, cycloalkyl or heterocycloalkyl radicals, for example phenyl or pyrrolidinyl radicals: the term “acyl” thus especially denotes, for example, formyl radicals and acetyl, propionyl, butanoyl, pentanoyl, hexanoyl, benzoyl and pyrrolidinylcarbonyl radicals,
the term “acylamino” denotes —C(O)—NH2, —C(O)—NH(alk) and —C(O)—N(alk)(alk) radicals: in these radicals, NH(alk) and N(alk)(alk) have the meanings given above,
the term “halogen atom” denotes chlorine, bromine, iodine or fluorine atoms and preferably the chlorine, bromine or fluorine atom,
the terms “aryl” and “heteroaryl” denote unsaturated radicals that are, respectively, carbocyclic and heterocyclic containing one or more hetero atoms, monocyclic or bicyclic that are not more than 12-membered,
the term “saturated or unsaturated carbocyclic or heterocyclic, monocyclic or bicyclic radical that is not more than 12-membered, containing one or more hetero atoms, which may be identical or different, chosen from O, N, NH and S, and which may contain a —C(O) member” includes the definitions which follow:
the term “saturated carbocyclic radical” especially denotes a cycloalkyl radical,
the term “cycloalkyl radical” denotes cyclopropyl, cyclobutyl, cyclopentyl and cyclohexyl radicals and most particularly cyclopentyl and cyclohexyl radicals,
the term “monocyclic heterocyclic radical” denotes a saturated or unsaturated 5- or 6-membered radical such that one or more of the members represents an oxygen, sulphur or nitrogen atom: such a heterocyclic or heterocycloalkyl radical thus denotes a carbocyclic radical interrupted with one or more hetero atoms chosen from oxygen, nitrogen and sulphur atoms, it being understood that the heterocyclic radicals can contain one or more hetero atoms chosen from oxygen, nitrogen and sulphur atoms and that, when these heterocyclic radicals contain more than one hetero atom, the hetero atoms of these heterocyclic radicals may be identical or different. Mention may be made especially of the dioxolane, dioxane, dithiolane, thiooxolane, thiooxane, morpholinyl, piperazinyl, piperazinyl substituted with a linear or branched alkyl radical containing not more than 4 carbon atoms, piperidyl, morpholinyl, thienyl such as 2-thienyl and 3-thienyl, furyl such as 2-furyl and 3-furyl, pyrimidinyl, pyridyl such as 2-pyridyl, 3-pyridyl and 4-pyridyl, pyrimidyl, pyrazolinyl, pyrrolyl, thiazolyl, isothiazolyl, diazolyl, thiadiazolyl, triazolyl, free or salified tetrazolyl, thiadiazolyl, thiatriazolyl, oxazolyl, oxadiazolyl or 3- or 4-isoxazolyl radical. Mention may be made most particularly of morpholinyl, thienyl such as 2-thienyl and 3-thienyl, furyl such as 2-furyl, tetrahydrofuryl, thienyl, tetrahydrothienyl, hexahydropyran, pyrrolyl, pyrrolinyl, pyrazolinyl, isoxazolyl, pyridyl, pyrrolidinyl, imidazolyl, pyrazolyl, pyridazinyl and oxodihydropyridazinyl radicals,
the term “bicyclic heterocyclic radical” denotes a saturated or unsaturated 8- to 12-membered radical such that one or more of the members represents an oxygen, sulphur or nitrogen atom, and especially fused heterocyclic groups containing at least one hetero atom chosen from sulphur, nitrogen and oxygen, for example benzothienyl such as 3-benzothienyl, benzothiazolyl, quinolyl, isoquinolyl, tetralone, benzofuryl, dihydrobenzofuran, ethylenedioxyphenyl, thianthrenyl, benzopyrrolyl, benzimidazolyl, benzoxazolyl, thionaphthyl, indolyl, purinyl, indazolyl, thienopyrazolyl, tetrahydroindazolyl, tetrahydrocyclopentapyrazolyl, dihydrofuropyrazolyl, tetrahydropyrrolopyrazolyl, oxotetrahydropyrrolopyrazolyl, tetrahydropyranopyrazolyl, tetrahydropyridinopyrazolyl or oxodihydropyridino-pyrazolyl,
the term “unsaturated carbocyclic radical” especially denotes phenyl and naphthyl radicals and more particularly a phenyl radical. It may be noted that a carbocyclic radical containing a —C(O) member is, for example, a tetralone radical,
the term “alkylphenyl” denotes a phenyl radical substituted with one or more linear or branched alkyl radicals as defined above, preferably containing not more than 4 carbon atoms.

The carboxyl radical(s) in the products of formula (I) may be salified (in ionized or salt form) or esterified with various reagents known to those skilled in the art, among which mention may be made, for example, of:

among the salification compounds, mineral bases such as, for example, one equivalent of sodium, potassium, lithium, calcium, magnesium or ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine and N-methylglucamine,

among the esterification compounds, alkyl radicals to form alkoxycarbonyl groups such as, for example, methoxycarbonyl, ethoxycarbonyl, tert-butoxycarbonyl or benzyloxycarbonyl, these alkyl radicals possibly being substituted with radicals chosen, for example, from halogen atoms, hydroxyl, alkoxy, acyl, acyloxy, alkylthio, amino or aryl radicals, such as, for example, chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may be, for example, the salts formed with hydrochloric acid, hydrobromic acid, hydriodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid and α,β-ethanedisulphonic acid, arylmonosulphonic acids such as benzenesulphonic acid, and aryldisulphonic acids.

It may be recalled that stereoisomerism may be defined in its broad sense as the isomerism of compounds having the same structural formulae, but whose various groups are arranged differently in space, such as especially in monosubstituted cyclohexanes in which the substituent may be in an axial or equatorial position, and the various possible rotational conformations of ethane derivatives. However, there is another type of stereoisomerism, due to the different spatial arrangements of attached substituents, either on double bonds or on rings, which is often referred to as geometrical isomerism or cis-trans isomerism. The term “stereoisomers” is used in the present invention in its broadest sense and thus concerns all of the compounds indicated above.

One subject of the present invention is thus the products of formula (I) as defined above corresponding to the formula (Ia):

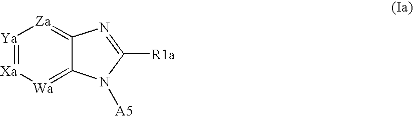

in which:
Xa represents C—R2a and Wa, Ya and Za, which may be identical or different, represent CH or CR3a;
R1a represents aryl or heteroaryl chosen from pyrazolyl, triazolyl or indazolyl radicals, all these radicals being optionally substituted with one or more radicals X1a, X2a or X3a chosen from H, halogen, OH, R4a, OR4a, NY1aY2a, S(O)nR4, —C(═O)NY1aY2a, —C(═O)OR4a, —N(R6)C(═O)R4a, —N(R6)SO2R4a, —N(R6)C(═O)NY1aY2a, —N(R6)C(═O)OR4a, —OC(═O)NY1aY2a and —OC(═O)R4a, —OS(O)nR4 and thienyl optionally substituted with an alkyl radical,
R2a and R3a are such that:
either R2a and R3a, which may be identical or different, represent H, R4a, halogen, OH, OR4a, C(═O)NY1Y2, —C(═O)OR4a, —C(═O)OH, and R3a represents alkyl, halogen and OR6,
or R2a represents H, R4a, halogen, OH, OR4a, C(═O)NY1Y2, —C(═O)OR4a, —C(═O)OH, and R3a represents alkyl, halogen and OR6,
or R2a and R3a together form an —O—CH2—O or —O—CH2—CH2-O— ring,
R4a represents alkyl, cycloalkyl, aryl, heteroaryl, heterocycloalkyl, heteroarylalkyl or arylalkyl, all these radicals being optionally substituted with one or more radicals chosen from aryl, OH, OR5a, C(═O)NY3aY4a, NY3aY4a and C(═O)OR6,
R5a represents alkyl, alkenyl, cycloalkyl, heterocycloalkyl, aryl, heteroaryl, arylalkyl, cycloalkylalkyl, heteroarylalkyl and heterocycloalkylalkyl,
R6 represents H and C1-C4 alkyl,
n represents an integer from 0 to 2,
Y1a and Y2a are such that: either Y1a and Y2a, which may be identical or different, represent H, alkyl, cycloalkyl, aryl and heteroaryl, all these radicals being optionally substituted with one or more radicals chosen from hydroxyl, —C(═O)—NY3aY4a, —C(═O)OR6 and NY3aY4a, or Y1a and Y2a form, together with the nitrogen atom to which they are attached, a cyclic amino radical,
Y3a and Y4a are such that: either Y3a and Y4a, which may be identical or different, represent hydrogen, alkyl, aryl, arylalkyl, cycloalkyl, heteroaryl or heteroarylalkyl, or Y3a and Y4a form, together with the nitrogen atom to which they are attached, a cyclic amino radical, A5 represents H or alkyl,
the said products of formula (Ia) being in any possible racemic, enantiomeric or diastereoisomeric isomer form, and also the addition salts with mineral and organic acids or with mineral bases.

One subject of the present invention is thus the products of formula (I) as defined above corresponding to the formula (IA):

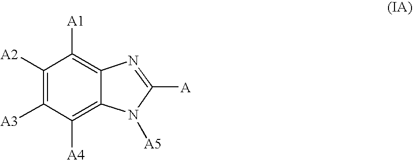

in which A represents a saturated heterocyclic radical which is either a 5- or 6-membered monocyclic radical or a bicyclic radical that is not more than 10-membered, these members being such that at least two of them represent a nitrogen atom and the others, which may be identical or different, represent a carbon member or a heterocycle member chosen from O, N and S, this heterocycle A being optionally substituted with one or more radicals XA1, XA2 or XA3 chosen from the values indicated in claim 1 for the radicals X1, X2 or X3,
   A1, A2, A3 and A4, which may be identical or different, are chosen from a hydrogen atom, halogen atoms and hydroxyl, alkyl, alkenyl, alkoxy, nitro, cyano, aryl, heteroaryl and aryloxy radicals, a carboxyl radical which is free, salified, esterified with an alkyl radical or amidated with a radical NA6A7 such that either A6 and A7, which may be identical or different, are chosen from a hydrogen atom and optionally substituted alkyl, alkoxyalkyl, phenoxyalkyl, aryl, arylalkyl, cycloalkyl, cycloalkylalkyl, heterocycloalkylalkyl and heteroarylalkyl radicals, or A6 and A7 form, together with the nitrogen atom to which they are attached, an optionally substituted 5- or 6-membered cyclic radical,
it being understood that two adjacent radicals among A1, A2, A3 and A4 can form, with the benzimidazole radical to which they are attached, a 5- to 6-membered carbon-based ring containing one or more hetero atoms, which may be identical or different, chosen from O, N and S,
   A5 represents a hydrogen atom or an alkyl radical,
   R6b represents hydrogen, alkyl, alkenyl, cycloalkyl, phenyl, phenylalkyl and cycloalkylalkyl,
   all the alkyl, alkenyl, aryl, heteroaryl, aryloxy, cycloalkyl and heterocycloalkyl radicals present in the above radicals being optionally substituted with one or more radicals chosen from halogen atoms and hydroxyl, cyano, alkyl, alkoxy, amino, alkylamino, dialkylamino, phenylamino, phenylalkylamino, acylamino (NH—COR6), —C(═O)OR6b, acyl —C(═O)R6b, hydroxyalkyl, carboxyalkyl, phenoxyalkyl, S(O)n-alk, S(O)n—NH2, S(O)n—NH(alk), S(O)n—N(alk)2, CF3, OCF3, NO2, CN, phenyl, itself optionally substituted with one or more halogen atoms, thienyl, phenoxy, phenylalkoxy, —C(═O)—NH2, —C(═O)—NH(alk) and C(═O)—N(alk)2 radicals,
   all the above alkyl, alkenyl, alkoxy and alkylthio radicals being linear or branched and containing not more than 4 carbon atoms,
   all the phenyl radicals of the above radicals furthermore being optionally substituted with a dioxole radical,
   n represents an integer from 0 to 2,
   the said products of formula (IA) being in any possible racemic, enantiomeric or diastereoisomeric isomer form, and also the addition salts with mineral and organic acids or with mineral and organic bases of the said products of formula (IA).

A subject of the present invention is thus the products of formula (I) as defined above, corresponding to the formula (IAa):

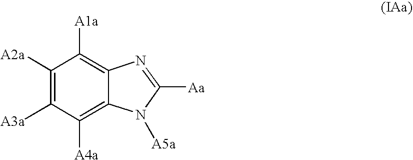

in which Aa represents a pyrazolyl, triazolyl or indazolyl radical, this heterocycle Aa being optionally substituted with one or more radicals XA1, XA2 or XA3 chosen from the values indicated in claim 1 for the radicals X1, X2 or X3,
A1a, A2a, A3a and A4a, which may be identical or different, are chosen from a hydrogen atom, halogen atoms, hydroxyl, alkyl, alkoxy, nitro, cyano, phenyl and phenoxy radicals, and a carboxyl radical which is free, salified, esterified with an alkyl radical or amidated with a radical NA6aA7a such that either A6a and A7a, which may be identical or different, are chosen from a hydrogen atom and alkyl, phenyl, phenylalkyl, cycloalkylalkyl, cycloalkyl, furylalkyl, thienylalkyl and pyridylalkyl radicals, or A6a and A7a form, together with the nitrogen atom to which they are attached, a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted,
it being understood that two adjacent radicals from among A1a, A2a, A3a and A4a may form, with the benzimidazole radical to which they are attached, an optionally substituted 5- to 6-membered carbon-based ring containing one or two oxygen atoms,
A5a represents a hydrogen atom or an alkyl radical, the phenyl and phenoxy radicals above being optionally substituted with one or more radicals chosen from halogen atoms and hydroxyl, cyano, trifluoromethyl, trifluoromethoxy, alkyl, alkoxy, amino, alkylamino, dialkylamino, phenylamino, phenylalkylamino, free, salified or esterified carboxyl, and dioxole radicals,
all the alkyl, alkoxy and alkylthio radicals above being linear or branched and containing not more than 6 carbon atoms,
   the said products of formula (IAa) being in any possible racemic, enantiomeric or diastereoisomeric isomer form, and also the addition salts with mineral and organic acids or with mineral and organic bases of the said products of formula (IAa).

The substituents X1, X2 and X3 as defined above are in particular such that one represents a hydrogen atom and the other two, which may be identical or different, are chosen from halogen atoms and OH, R4a, OR4a, CF3, OCF3, NO2, CN, NY1aY2a, acylamino (NH—COR6b), S(O)n-alk, S(O)n—NH2, S(O)n—NH(alk), S(O)n—N(alk)2, —C(═O)—NH2, —C(═O)—NH(alk), C(═O)—N(alk)2, -, —C(═O)OR4a, —N(R6b)C(═O)R4a, —N(R6b)SO2R4a, —N(R6b)C(═O)NY1aY2a, —N(R6b)C(═O)OR4a, —OC(═O)NY1aY2a and thienyl radicals, the thienyl radical being optionally substituted with an alkyl radical,

R4a, Y1a, Y2a and R6b having the values defined above and alk representing a linear or branched alkyl radical including not more than 6 carbon atoms and optionally substituted as indicated above.

Tables I, II and III described below give examples of products illustrating the present invention, with in particular substituents chosen from the values of X1, X2 and X3 as defined above.

All the alkylthio radicals are such that the sulphur atom is optionally oxidized to sulphone or sulphoxide with one or two oxygen atoms.

The subject of the present invention is thus the products of formula (I) as defined above in which the substituents of the said products of formula (I) have the values indicated in any one of the preceding claims and in which the aryl radicals represent the phenyl and naphthyl radicals; the heteroaryl radicals represent the furyl, thienyl, benzothienyl, thianthrenyl, pyridyl, pyrazolyl, benzimidazolyl, benzofuran, isobenzofuran and dihydrobenzofuran radicals; the cycloalkyl radicals represent a cyclopropyl, cyclobutyl, cyclopentyl or cyclohexyl radical; the heterocycloalkyl radicals represent the hexahydropyran, piperidyl or morpholino radicals; the heterocycloalkylalkyl radicals represent the hexahydropyranalkyl, piperidylalkyl and morpholinoalkyl radicals; the arylalkyl radicals represent the phenylalkyl, ethylenedioxyphenylalkyl and naphthylalkyl radicals; the heteroarylalkyl radicals represent the thienylalkyl, pyridylalkyl, furylalkyl, pyrazolylalkyl, benzothienylalkyl, dihydrobenzofuranalkyl and benzimidazolalkyl radicals; the aryloxy radicals represent the phenoxy and naphthyloxy radicals; the arylalkoxy radicals represent the phenylalkoxy and naphthylalkoxy radical; and the aryloxyalkyl radicals represent the phenoxyalkyl radical; all these radicals being optionally substituted as indicated in any one of the preceding claims.

One subject of the present invention is, more particularly, the products of formula (I) as defined above corresponding to the formula (IA):

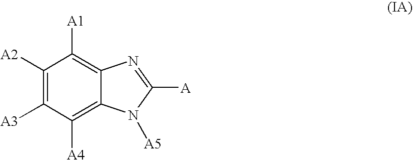

in which A represents a saturated heterocyclic radical which is either a 5- or 6-membered monocyclic radical or a bicyclic radical that is not more than 10-membered, these members being such that at least two of them represent a nitrogen atom and the others, which may be identical or different, represent a carbon member or a heterocycle member chosen from O, N and S, this heterocycle A optionally being substituted with one or more radicals XA1, XA2 or XA3 chosen from halogen atoms, alkyl, alkoxy or alkylthio radicals or thienyl radicals optionally substituted with an alkyl radical,
A1, A2, A3 and A4, which may be identical or different, are chosen from a hydrogen atom, halogen atoms and hydroxyl, alkyl, alkoxy, nitro, cyano, phenyl and phenoxy radicals, a carboxyl radical which is free, salified, esterified with an alkyl radical or amidated with a radical NA6A7 such that either A6 and A7, which may be identical or different, are chosen from a hydrogen atom and alkyl, phenyl, phenylalkyl, cycloalkylalkyl, cycloalkyl and heteroarylalkyl radicals, or A6 and A7 form, together with the nitrogen atom to which they are attached, a 5- or 6-membered cyclic radical,
it being understood that two adjacent radicals among A1, A2, A3 and A4 can form, with the benzimidazole radical to which they are attached, a 5- to 6-membered carbon-based ring containing one or more hetero atoms, which may be identical or different, chosen from O, N and S,
A5 represents a hydrogen atom or an alkyl radical,
all the phenyl, phenoxy, cycloalkyl and heteroarylalkyl radicals above being optionally substituted with one or more radicals chosen from halogen atoms and hydroxyl, cyano, trifluoromethyl, trifluoromethoxy, alkyl, alkoxy, amino, alkylamino, dialkylamino, phenylamino, phenylalkylamino, free, salified or esterified carboxyl, and dioxole radicals,
all the alkyl, alkoxy and alkylthio radicals above being linear or branched and containing not more than 6 carbon atoms,
the said products of formula (IA) being in any possible racemic, enantiomeric or diastereoisomeric isomer form, and also the addition salts with mineral and organic acids or with mineral and organic bases of the said products of formula (IA).

A subject of the present invention is also, more particularly, the products of formula (I) as defined above, corresponding to the formula (IAb):

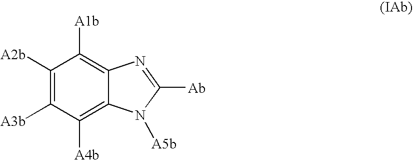

in which Ab represents a pyrazolyl or indazolyl radical optionally substituted with one or two radicals chosen from halogen atoms and OH, alkyl, alkynyl, —OR6b (including alkoxy), —COR6b, —O—COR6b, —OS(O)nR6b, —O(CH2)n—CO—R6b, phenyl, phenylalkyl, CF3, OCF3, NO2, CN, NY1bY2b, —NH—C(═O)NY1bY2b, acylamino (NH—CO—R6b), S(O)n-alk, S(O)n—NY1bY2b, —C(═O)—NY1bY2b, —C(═O)OR6b, —NH—C(═O)R6b, —NH—S(O)nR6b, —NH—C(═O)OR6b, —N(R6b)C(═O)NY1bY2b, —OC(═O)NY1bY2b and thienyl radicals, all these radicals being optionally substituted,
   with NY1bY2b such that either Y1b and Y2b, which may be identical or different, are chosen from hydrogen and optionally substituted alkyl, cycloalkyl, cycloalkylalkyl, phenyl, naphthyl, phenoxy, phenylalkyl, phenylalkylthio and naphthylalkyl or Y1b and Y2b form, together with the nitrogen atom to which they are attached, a piperidyl, hexahydrofuran, morpholinyl or morpholinylalkyl radical,
A1b, A2b, A3b and A4b, which may be identical or different, are chosen from a hydrogen atom, halogen atoms, hydroxyl, alkyl, alkenyl, —OR6b (including alkoxy), —CO—R6b, —O—COR6b, —OS(O)nR6b, —O(CH2)n—CO—R6b, nitro, cyano, furyl, thienyl, benzothienyl, naphthyl, thianthrenyl, phenyl and phenoxy radicals and a carboxyl radical which is free, salified, esterified with an alkyl radical or amidated with a radical NA6bA7b such that either A6b and A7b, which may be identical or different, are chosen from hydrogen and alkyl, alkoxyalkyl, phenoxyalkyl, phenyl, phenylalkyl, cycloalkylalkyl, cycloalkyl, furylalkyl, naphthylalkyl, thienylalkyl, piperidylalkyl, pyridylalkyl, benzothienylalkyl, pyrazolylalkyl, dihydrobenzofuranalkyl, hexahydropyranalkyl, ethylenedioxyphenylalkyl and benzimidazolylalkyl radicals, all these radicals being optionally substituted, or A6b and A7b form, together with the nitrogen atom to which they are attached, a pyrrolidinyl, morpholino or piperazinyl radical, the piperazinyl radical being optionally substituted on the second nitrogen atom with an alkyl radical itself optionally substituted,
it being understood that two adjacent radicals among A1b, A2b, A3b and A4b can form, with the benzimidazole radical to which they are attached, an optionally substituted 4,5-ethylenedioxybenzimidazole radical or an optionally substituted 4,5-methylenedioxybenzimidazole radical,
A5b represents a hydrogen atom,
   all the above radicals containing alkyl, alkenyl, phenyl, phenoxy, furyl, thienyl, piperidyl, pyridyl, pyrazolyl and benzimidazolyl being optionally substituted with one or more radicals chosen from halogen atoms and hydroxyl, cyano, alkyl, alkoxy, amino, alkylamino, dialkylamino, phenylamino, phenylalkylamino, acylamino (NH—COR6b), —C(═O)OR6b, acyl —C(═O)R6b, hydroxyalkyl, carboxyalkyl, phenoxyalkyl, S(O)n-alk, S(O)n—NH2, S(O)n—NH(alk), S(O)n—N(alk)2, CF3, OCF3, NO2, CN, phenyl, itself optionally substituted with one or more halogen atoms, thienyl, phenoxy, phenylalkoxy, —C(═O)—NH2, —C(═O)—NH(alk) and C(═O)—N(alk)2 radicals,
   with n represents an integer from 0 to 2,
   and R6b represents hydrogen, alkyl, alkenyl, cycloalkyl, phenyl, pyridyl, thienyl, naphthyl, isoxazole, adamentyl, quinoline, quinolone, dihydroquinolone, —NH-phenyl, phenylalkyl and cycloalkylalkyl, all these radicals being optionally substituted with a morpholino, piperidyl or phenyl radical itself optionally substituted with one or more radicals chosen from halogen atoms and the cyano, CF3, OCF3, alkyl, phenyl-S(O)n-alk-phenyl, alkoxy, NH2, NHalk, N(alk)2, SO2NH2; SO2Nalk or SO2N(alk)2 radical,
all the alkyl, alkenyl, alkoxy and alkylthio radicals above being linear or branched and containing not more than 10 carbon atoms, all the phenyl radicals of the above radicals furthermore being optionally substituted with a dioxole radical,
   the said products of formula (IAb) being in any possible racemic, enantiomeric or diastereomeric isomer form, and also the addition salts with mineral and organic acids or with mineral and organic bases of the said products of formula (IAb)).

One subject of the present invention is thus in particular the products of formula (I) as defined above corresponding to the formula (IAb) in which Ab represents a pyrazolyl or indazolyl radical optionally substituted with one or two radicals chosen from halogen atoms and OH, alkyl, alkynyl, alkoxy, phenyl, phenylalkyl, CF3, OCF3, NO2, CN, NY1bY2b, —NH—C(═O)NY1bY2b, acylamino (NH—CO—R6b), S(O)n-alk, S(O)n—NY1bY2b, —C(═O)—NY1bY2b, —C(═O)OR6b, —NH—C(═O)R6b, —NH—S(O)nR6b, —NH—C(═O)OR6b, —N(R6b)C(═O)NY1bY2b, —OC(═O)NY1bY2b and thienyl radicals which are optionally substituted,

with NY1bY2b such that either Y1b and Y2b, which may be identical or different, are chosen from hydrogen and optionally substituted alkyl, cycloalkyl, cycloalkylalkyl, phenyl, naphthyl, phenoxy, phenylalkyl, phenylalkylthio and naphthylalkyl or Y1b and Y2b form, together with the nitrogen atom to which they are attached, a piperidyl, hexahydrofuran, morpholinyl or morpholinylalkyl radical,
A1b, A2b, A3b and A4b, which may be identical or different, are chosen from a hydrogen atom, halogen atoms, hydroxyl, alkyl, alkenyl, alkoxy, nitro, cyano, furyl, thienyl, benzothienyl, naphthyl, thianthrenyl, phenyl and phenoxy radicals and a carboxyl radical which is free, salified, esterified with an alkyl radical or amidated with a radical NA6bA7b such that either A6b and A7b, which may be identical or different, are chosen from hydrogen and alkyl, alkoxyalkyl, phenoxyalkyl, phenyl, phenylalkyl, cycloalkylalkyl, cycloalkyl, furylalkyl, naphthylalkyl, thienylalkyl, piperidylalkyl, pyridylalkyl, benzothienylalkyl, pyrazolylalkyl, dihydrobenzofuranalkyl, hexahydropyranalkyl, ethylenedioxyphenylalkyl and benzimidazolylalkyl radicals, all these radicals being optionally substituted, or A6b and A7b form, together with the nitrogen atom to which they are attached, a pyrrolidinyl, morpholino or piperazinyl radical, the piperazinyl radical being optionally substituted on the second nitrogen atom with an alkyl radical itself optionally substituted,
it being understood that two adjacent radicals among A1b, A2b, A3b and A4b can form, with the benzimidazole radical to which they are attached, an optionally substituted 4,5-ethylenedioxybenzimidazole radical or an optionally substituted 4,5-methylenedioxybenzimidazole radical, A5b represents a hydrogen atom,
   all the above radicals containing alkyl, alkenyl, phenyl, phenoxy, furyl, thienyl, piperidyl, pyridyl, pyrazolyl and benzimidazolyl being optionally substituted with one or more radicals chosen from halogen atoms and hydroxyl, cyano, alkyl, alkoxy, amino, alkylamino, dialkylamino, phenylamino, phenylalkylamino, acylamino (NH—COR6b), —C(═O)OR6b, acyl —C(═O)R6b, hydroxyalkyl, carboxyalkyl, phenoxyalkyl, S(O)n-alk, S(O)n—NH2, S(O)n—NH(alk), S(O)n—N(alk)2, CF3, OCF3, NO2, CN, phenyl, itself optionally substituted with one or more halogen atoms, thienyl, phenoxy, phenylalkoxy, —C(═O)—NH2, —C(═O)—NH(alk) and C(═O)—N(alk)2 radicals,
   with n represents an integer from 0 to 2,
   and R6b represents hydrogen, alkyl, alkenyl, cycloalkyl, phenyl, phenylalkyl and cycloalkylalkyl,
all the alkyl, alkenyl, alkoxy and alkylthio radicals above being linear or branched and containing not more than 10 carbon atoms,
all the phenyl radicals of the above radicals furthermore being optionally substituted with a dioxole radical,
   the said products of formula (IAb) being in any possible racemic, enantiomeric or diastereoisomeric isomer form, and also the addition salts with mineral and organic acids or with mineral and organic bases of the said products of formula (IAb)).

A subject of the present invention is thus in particular the products of formula (I) as defined above corresponding to the formula (IAb) in which Ab represents a pyrazolyl radical substituted with one or two radicals such that one is chosen from hydrogen, halogen atoms and alkyl, alkynyl, —COR6b, phenyl, phenylalkyl, CF3, NO2, CN, NY1bY2b, —NH—C(═O)NY1bY2b, NH—CO—R6b, S(O)n-alk, S(O)n—NY1bY2b, —C(═O)—NY1bY2b, —C(═O)OR6b, —NH—C(═O)R6b, —NH—S(O)nR6b, —NH—C(═O)OR6b, —N(R6b)C(═O)NY1bY2b and thienyl radicals, all these radicals being optionally substituted,

and the other is chosen from OH, —OR6b, —O—COR6b, —OS(O)nR6b, —O(CH2)n—CO—R6b and —OC(═O)NY1bY2b radicals, all these radicals being optionally substituted,
with NY1bY2b such that Y1b and Y2b, which may be identical or different, are chosen from hydrogen and optionally substituted alkyl, cycloalkyl, cycloalkylalkyl, phenyl, naphthyl, phenoxy, phenylalkyl, phenylalkylthio and naphthylalkyl or Y1b and Y2b form, together with the nitrogen atom to which they are attached, a piperidyl, hexahydrofuran, morpholinyl or morpholinylalkyl radical,
A1b, A2b, A3b and A4b, which may be identical or different, are such that two of them represent hydrogen and the other two, which may be identical or different, are chosen from a hydrogen atom, halogen atoms, hydroxyl, alkyl, alkenyl, —OR6b (including alkoxy), —CO—R6b, —O—COR6b, —OS(O)nR6b, —O(CH2)n—CO—R6b, nitro, cyano, furyl, thienyl, benzothienyl, naphthyl, thianthrenyl, phenyl and phenoxy radicals and a carboxyl radical which is free, salified, esterified with an alkyl radical or amidated with a radical NA6bA7b such that either A6b and A7b, which may be identical or different, are chosen from hydrogen and alkyl, alkoxyalkyl, phenoxyalkyl, phenyl, phenylalkyl, cycloalkylalkyl, cycloalkyl, furylalkyl, naphthylalkyl, thienylalkyl, piperidylalkyl, pyridylalkyl, benzothienylalkyl, pyrazolylalkyl, dihydrobenzofuranalkyl, hexahydropyranalkyl, ethylenedioxyphenylalkyl and benzimidazolylalkyl radicals, all these radicals being optionally substituted, or A6b and A7b form, together with the nitrogen atom to which they are attached, a pyrrolidinyl, morpholino or piperazinyl radical, the piperazinyl radical being optionally substituted on the second nitrogen atom with an alkyl radical itself optionally substituted,
A5b represents a hydrogen atom,
   all the above radicals containing alkyl, alkenyl, phenyl, phenoxy, furyl, thienyl, piperidyl, pyridyl, pyrazolyl and benzimidazolyl being optionally substituted with one or more radicals chosen from halogen atoms and hydroxyl, cyano, alkyl, alkoxy, amino, alkylamino, dialkylamino, phenylamino, phenylalkylamino, acylamino (NH—COR6b), —C(═O)OR6b, acyl —C(═O)R6b, hydroxyalkyl, carboxyalkyl, phenoxyalkyl, S(O)n-alk, S(O)n—NH2, S(O)n—NH(alk), S(O)n—N(alk)2, CF3, OCF3, NO2, CN, phenyl, itself optionally substituted with one or more halogen atoms, thienyl, phenoxy, phenylalkoxy, —C(═O)—NH2, —C(═O)—NH(alk) and C(═O)—N(alk)2 radicals,
   with n represents an integer from 0 to 2,
   and R6b represents hydrogen, alkyl, alkenyl, cycloalkyl, phenyl, pyridyl, thienyl, naphthyl, isoxazole, adamentyl, quinoline, quinolone, dihydroquinolone, —NH-phenyl, phenylalkyl and cycloalkylalkyl, all these radicals being optionally substituted with a morpholino, piperidyl or phenyl radical itself optionally substituted with one or more radicals chosen from halogen atoms and the cyano, CF3, OCF3, alkyl, phenyl-S(O)n-alk-phenyl, alkoxy, NH2, NHalk, N(alk)2, SO2NH2, SO2Nalk or SO2N(alk)2 radical,
all the alkyl, alkenyl, alkoxy and alkylthio radicals above being linear or branched and containing not more than 10 carbon atoms,
all the phenyl radicals of the above radicals furthermore being optionally substituted with a dioxole radical,
   the said products of formula (IAb) being in any possible racemic, enantiomeric or diastereoisomeric isomer form, and also the addition salts with mineral and organic acids or with mineral and organic bases of the said products of formula (IAb)).

A subject of the present invention is thus in particular the products of formula (I) as defined above corresponding to the formula (IAb) in which Ab represents a pyrazolyl or indazolyl radical optionally substituted with one or more radicals chosen from halogen atoms and alkyl, alkoxy and thienyl radicals,
A1b, A2b, A3b and A4b, which may be identical or different, are chosen from a hydrogen atom; halogen atoms; radicals of the following types: hydroxyl, alkyl, alkenyl optionally substituted with phenyl itself optionally substituted with one or more halogen atoms, alkoxy, nitro, cyano, furyl, thienyl optionally substituted with acyl COalk, benzothienyl, naphthyl, thianthrenyl, phenyl and phenoxy which are optionally substituted; and a carboxyl radical which is free, salified, esterified with an alkyl radical or amidated with a radical NA6bA7b such that either A6b and A7b, which may be identical or different, are chosen from hydrogen and radicals of the following types: alkyl, alkoxyalkyl containing not more than 6 carbon atoms, phenoxyalkyl optionally substituted with acylamino NH—C(O)alk, phenyl, optionally substituted phenylalkyl, cycloalkylalkyl, cycloalkyl, furylalkyl optionally substituted with one or more alkyl radicals, naphthylalkyl, thienylalkyl optionally substituted with alkyl or thienyl, piperidylalkyl optionally substituted with a carboxyl radical which is free, salified or esterified with an alkyl radical, pyridylalkyl optionally substituted with one or more radicals chosen from halogen and CF3, benzothienylalkyl, pyrazolylalkyl optionally substituted with one or more alkyl radicals, dihydrobenzofuranalkyl, hexahydropyranalkyl, ethylenedioxyphenylalkyl, and benzimidazolylalkyl optionally substituted with one or more alkyl radicals,
or A6b and A7b form, together with the nitrogen atom to which they are attached, a pyrrolidinyl, morpholino or piperazinyl radical, the piperazinyl radical being optionally substituted on the second nitrogen atom with an alkyl radical,
it being understood that two adjacent radicals among A1b, A2b, A3b and A4b can form, with the benzimidazole radical to which they are attached, an optionally substituted 4,5-ethylenedioxybenzimidazole radical or an optionally substituted 4,5-methylenedioxybenzimidazole radical,
R5a represents a hydrogen atom,
   the phenyl, phenoxy and phenylalkyl radicals above being optionally substituted with one or more radicals chosen from halogen atoms, hydroxyl, cyano, alkyl, alkoxy, amino, alkylamino, dialkylamino, phenylamino, phenylalkylamino and NH—COalk radicals, a carboxyl radical which is free, salified or esterified with an alkyl radical, and hydroxyalkyl, carboxyalkyl, phenoxyalkyl, alkylthio, SO2alk, SO2NH2, SO2—NH(alk), SO2—N(alk)2, CF3, OCF3, NO2, CN, phenyl, itself optionally substituted with one or more halogen atoms, thienyl, phenoxy, phenylalkoxy, —C(═O)—NH2, —C(═O)—NH(alk), C(═O)—N(alk)2 and C(O)CH3 radicals,
   all the alkyl or alk, alkenyl, alkoxy and alkylthio radicals above being linear or branched and containing not more than 4 carbon atoms,
   all the phenyl radicals of the above radicals furthermore being optionally substituted with a dioxole radical,
   the said products of formula (IAb) being in any possible racemic, enantiomeric or diastereoisomeric isomer form, and also the addition salts with mineral and organic acids or with mineral and organic bases of the said products of formula (IAb)).

A subject of the present invention is thus in particular the products of formula (I) as defined above corresponding to the formula (IAb) in which Ab, A1b, A2b, A3b, A4b and A5b have the meanings as defined above,
and when one of A1b, A2b, A3b and A4b represents a carboxyl radical amidated with a radical NA6bA7b, then either one of A6b and A7b represents a hydrogen atom or an alkyl radical and the other of A6b and A7b is chosen from the values defined for A6b and A7b, or A6b and A7b form, together with the nitrogen atom to which they are attached, a 5- or 6-membered cyclic radical,
the other substituents of the said products of formula (I) having the values as defined above,
   the said products of formula (IAb) being in any possible racemic, enantiomeric or diastereoisomeric isomer form, and also the addition salts with mineral and organic acids or with mineral and organic bases of the said products of formula (IAb)).

A subject of the present invention is thus in particular the products of formula (I) as defined above in which X, W, Y and Z are such that two or three of them represent CH and the others are chosen from the values of CR2 or CR3 and, if appropriate, can form a dioxole radical,

R2, R3 and the other substituents of the said products of formula (I) having the values defined above,
the said products of formula (I) being in any possible racemic, enantiomeric or diastereoisomeric isomer form, and also the addition salts with mineral and organic acids or with mineral and organic bases of the said products of formula (I).

The present invention thus relates in particular to the products of formula (IA) as defined above in which A1, A2, A3 and A4 are such that two or three of them represent a hydrogen atom and the others are chosen from the values of A1, A2, A3 and A4 and, if appropriate, can form a dioxole radical,

the other substituents of the products of formula (IA) having the values as defined above,
the said products of formula (IA) being in any possible racemic, enantiomeric or diastereoisomeric isomer form, and also the addition salts with mineral and organic acids or with mineral and organic bases of the said products of formula (IA).

A subject of the present invention is also, more particularly, the products of formula (I) as defined above, corresponding to the formula (IAa):

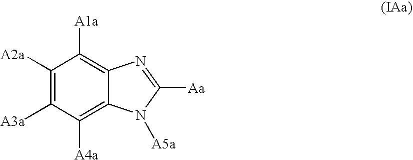

in which Aa represents a pyrazolyl, triazolyl or indazolyl radical, this heterocycle Aa being optionally substituted with one or more radicals XA1, XA2 or XA3 chosen from halogen atoms, alkyl, alkoxy or alkylthio radicals and thienyl radicals optionally substituted with an alkyl radical,
A1a, A2a, A3a and A4a, which may be identical or different, are chosen from a hydrogen atom, halogen atoms, hydroxyl, alkyl, alkoxy, nitro, cyano, phenyl and phenoxy radicals, and a carboxyl radical which is free, salified, esterified with an alkyl radical or amidated with a radical NA6aA7a such that either A6a and A7a, which may be identical or different, are chosen from a hydrogen atom and alkyl, phenyl, phenylalkyl, cycloalkylalkyl, cycloalkyl, furylalkyl, thienylalkyl and pyridylalkyl radicals, or A6a and A7a form, together with the nitrogen atom to which they are attached, a pyrrolidinyl, pyrazolidinyl, pyrazolinyl, piperidyl, morpholino or piperazinyl radical optionally substituted on the second nitrogen atom with an alkyl or phenyl radical, which are themselves optionally substituted, it being understood that two adjacent radicals from among A1a, A2a, A3a and A4a may form, with the benzimidazole radical to which they are attached, an optionally substituted 5- to 6-membered carbon-based ring containing one or two oxygen atoms,
A5a represents a hydrogen atom or an alkyl radical,
the phenyl and phenoxy radicals above being optionally substituted with one or more radicals chosen from halogen atoms and hydroxyl, cyano, trifluoromethyl, trifluoromethoxy, alkyl, alkoxy, amino, alkylamino, dialkylamino, phenylamino, phenylalkylamino, free, salified or esterified carboxyl, and dioxole radicals,
all the alkyl, alkoxy and alkylthio radicals above being linear or branched and containing not more than 6 carbon atoms,
the said products of formula (IAa) being in any possible racemic, enantiomeric or diastereoisomeric isomer form, and also the addition salts with mineral and organic acids or with mineral and organic bases of the said products of formula (IAa).

One subject of the present invention is, more particularly, the products of formula (I) as defined above in which R represents a pyrazolyl or indazolyl radical, the other substituents having the values indicated above or below.

Among the preferred products that are particularly noted are the products of formula (IAa) in which

Aa represents a pyrazolyl or indazolyl radical optionally substituted as indicated above and below, A1a, A2a, A3a and A4a are chosen from the following values:
Ala represents hydrogen or carboxyl or forms a ring with the adjacent member A2a A4a represents hydrogen or carboxyl or forms a ring with the adjacent member A3a
A2a represents a carboxyl radical that is free, salified, esterified with an optionally substituted alkyl radical or an amidated carboxyl as indicated above or below,
A2a and A3a represent two optionally substituted alkyl radicals,
A5a represents hydrogen.

One subject of the present invention is, even more particularly, the products of formula (I) as defined above, corresponding to the formula (IAb):

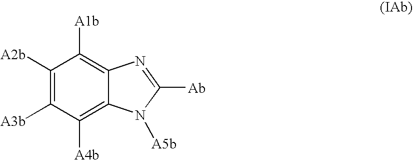

in which Ab represents a pyrazolyl or indazolyl radical optionally substituted with one or more radicals chosen from halogen atoms and alkyl, alkoxy and thienyl radicals,

A1b, A2b, A3b and A4b, which may be identical or different, are chosen from a hydrogen atom, halogen atoms, hydroxyl, alkyl and alkoxy, nitro, cyano, phenyl and phenoxy radicals, and a carboxyl radical that is free, salified, esterified with an alkyl radical or amidated with a radical NA6bA7b such that either A6b and A7b, which may be identical or different, are chosen from alkyl, phenyl, phenylalkyl, cycloalkylalkyl, cycloalkyl and furylalkyl radicals, or A6b and A7b form, together with the nitrogen atom to which they are attached, a pyrrolidinyl, morpholino or piperazinyl radical optionally substituted on the second nitrogen atom with an alkyl radical,

it being understood that two adjacent radicals from among A1b, A2b, A3b and A4b may form, with the benzimidazole radical to which they are attached, an optionally substituted 4,5-ethylenedioxybenzimidazole radical or 4,5-methylenedioxybenzimidazole radical,
A5b represents a hydrogen atom,
the phenyl and phenoxy radicals above being optionally substituted with one or more radicals chosen from halogen atoms and hydroxyl, cyano, alkyl, alkoxy, amino, alkylamino, dialkylamino, phenylamino, phenylalkylamino and free, salified or esterified carboxyl radicals,
all the alkyl, alkoxy and alkylthio radicals above being linear or branched and containing not more than 4 carbon atoms,
the said products of formula (IAb) being in any possible racemic, enantiomeric or diastereoisomeric isomer from, and also the addition salts with mineral and organic acids or with mineral and organic bases of the said products of formula (Iab).

One subject of the present invention is, most particularly, the products of formula (I) as defined above, corresponding to the following formulae:
2-(1H-indazol-3-yl)-1H-benzimidazole-5-carboxylic acid benzylamide
2-(1H-indazol-3-yl)-1H-benzimidazole-5-carboxylic acid N-methylamide
2-(1H-indazol-3-yl)-1H-benzimidazole-5-carboxylic acid N-ethylamide
2-(1H-indazol-3-yl)-1H-benzimidazole-5-carboxylic acid N-isopropylamide
2-(1H-indazol-3-yl)-1H-benzimidazole-5-carboxylic acid N-phenylamide
2-(1H-indazol-3-yl)-1H-benzimidazole-5-carboxylic acid N-phenethylamide
2-(1H-indazol-3-yl)-1H-benzimidazole-5-carboxylic acid N-morpholinoamide
2-(1H-indazol-3-yl)-1H-benzimidazole-5-carboxylic acid
N—(N′-methylpiperazino)amide
2-(1H-indazol-3-yl)-1H-benzimidazole-5-carboxylic acid N-pyrrolidinoamide
2-(1H-indazol-3-yl)-1H-benzimidazole-5-carboxylic acid N-(isobutyl)amide
2-(1H-indazol-3-yl)-1H-benzimidazole-5-carboxylic acid N-(cyclohexylmethyl)amide
2-(1H-indazol-3-yl)-1H-benzimidazole-5-carboxylic acid N-(2-furfuryl)amide
2-(1H-indazol-3-yl)-1H-benzimidazole-5-carboxylic acid N-benzyl-N-methylamide
methyl 2-(1H-indazol-3-yl)-3H-benzimidazole 5-carboxylate
5,6-dimethyl-2-(1H-indazol-3-yl)-1H-benzimidazole
5-methoxy-2-(1H-indazol-3-yl)-1H-benzimidazole
2-(1H-indazol-3-yl)-3H-benzimidazole-4-carboxylic acid
5-bromo 2-(1H-indazol-3-yl)-3H-benzimidazole
2-(5-ethoxy-2H-pyrazol-3-yl)-1H-benzimidazole-4-carboxylic acid
5,6-dimethyl-2-(5-methyl-2H-pyrazol-3-yl)-1H-benzimidazole
5,6-dimethyl-2-(5-thiophen-2-yl-2H-pyrazol-3-yl)-1H-benzimidazole
2-(4-bromo-2H-pyrazol-3-yl)-5,6-dimethyl-1H-benzimidazole
2-(5-ethyl-2H-pyrazol-3-yl)-5,6-dimethyl-1H-benzimidazole
2-(5-ethyl-2H-pyrazol-3-yl)-4,5-ethylenedioxy-1H-benzimidazole
2-(5-ethyl-2H-pyrazol-3-yl)-5-methoxy-1H-benzimidazole
2-(5-ethyl-2H-pyrazol-3-yl)-4-hydroxy-1H-benzimidazole
2-(5-ethyl-2H-pyrazol-3-yl)-5-bromo-1H-benzimidazole
2-(1H-indazol-3-yl)-1H-benzimidazole-5-carboxylic acid 4-(aminosulphonyl)benzylamide
2-(1H-indazol-3-yl)-1H-benzimidazole-5-carboxylic acid 4-bromobenzylamide
2-(1H-indazol-3-yl)-1H-benzimidazole-5-carboxylic acid 4-(methanesulphonyl)benzylamide
2-(1H-indazol-3-yl)-1H-benzimidazole-5-carboxylic acid 4-nitrobenzylamide
2-(1H-indazol-3-yl)-1H-benzimidazole-5-carboxylic acid 2-methylbenzylamide
2-(1H-indazol-3-yl)-1H-benzimidazole-5-carboxylic acid (6-chloropyridin-3-ylmethyl)amide
2-(1H-indazol-3-yl)-1H-benzimidazole-5-carboxylic acid (2,3-dihydrobenzofuran-5-ylmethyl)amide
2-(1H-indazol-3-yl)-1H-benzimidazole-5-carboxylic acid 2-(methylsulphanyl)benzylamide
2-(1H-indazol-3-yl)-1H-benzimidazole-5-carboxylic acid (benzo[b]thiophen-3-ylmethyl)amide
2-(1H-indazol-3-yl)-1H-benzimidazole-5-carboxylic acid 3-methylbenzylamide
2-(1H-indazol-3-yl)-1H-benzimidazole-5-carboxylic acid 3-chlorobenzylamide
2-(1H-indazol-3-yl)-3H-benzimidazole-4-carboxylic acid 2-(methylsulphanyl)benzylamide

One subject of the present invention is, most particularly, the products of formula (I) as defined above, corresponding to the following formulae:
2-(1H-indazol-3-yl)-1H-benzimidazole-5-carboxylic acid benzylamide
2-(1H-indazol-3-yl)-1H-benzimidazole-5-carboxylic acid N-methylamide
2-(1H-indazol-3-yl)-1H-benzimidazole-5-carboxylic acid N-ethylamide
2-(1H-indazol-3-yl)-1H-benzimidazole-5-carboxylic acid N-isopropylamide
2-(1H-indazol-3-yl)-1H-benzimidazole-5-carboxylic acid N-phenylamide
2-(1H-indazol-3-yl)-1H-benzimidazole-5-carboxylic acid N-phenethylamide
2-(1H-indazol-3-yl)-1H-benzimidazole-5-carboxylic acid N-morpholinoamide
2-(1H-indazol-3-yl)-1H-benzimidazole-5-carboxylic acid N—(N′-methyl-piperazino)amide
2-(1H-indazol-3-yl)-1H-benzimidazole-5-carboxylic acid N-pyrrolidinoamide
2-(1H-indazol-3-yl)-1H-benzimidazole-5-carboxylic acid N-(isobutyl)amide
2-(1H-indazol-3-yl)-1H-benzimidazole-5-carboxylic acid N-(cyclohexylmethyl)amide
2-(1H-indazol-3-yl)-1H-benzimidazole-5-carboxylic acid N-(2-furfuryl)amide
2-(1H-indazol-3-yl)-1H-benzimidazole-5-carboxylic acid N-benzyl-N-methylamide
5-methoxy-2-(1H-indazol-3-yl)-1H-benzimidazole
2-(5-ethyl-2H-pyrazol-3-yl)-5,6-dimethyl-1H-benzimidazole
2-(5-ethyl-2H-pyrazol-3-yl)-4,5-ethylenedioxy-1H-benzimidazole
2-(5-ethyl-2H-pyrazol-3-yl)-5-methoxy-1H-benzimidazole
2-(5-ethyl-2H-pyrazol-3-yl)-4-hydroxy-1H-benzimidazole
2-(5-ethyl-2H-pyrazol-3-yl)-5-bromo-1H-benzimidazole.

One subject of the present invention is, most particularly, the products of formula (I) as defined above, corresponding to the following formulae:
2-(1H-indazol-3-yl)-1H-benzimidazole-5-carboxylic acid 4-(aminosulphonyl)benzylamide
2-(1H-indazol-3-yl)-1H-benzimidazole-5-carboxylic acid 4-bromobenzylamide
2-(1H-indazol-3-yl)-1H-benzimidazole-5-carboxylic acid 4-(methanesulphonyl)benzylamide
2-(1H-indazol-3-yl)-1H-benzimidazole-5-carboxylic acid 4-nitrobenzylamide
2-(1H-indazol-3-yl)-1H-benzimidazole-5-carboxylic acid 2-methylbenzylamide
2-(1H-indazol-3-yl)-1H-benzimidazole-5-carboxylic acid (6-chloropyridin-3-ylmethyl)amide
2-(1H-indazol-3-yl)-1H-benzimidazole-5-carboxylic acid (2,3-dihydrobenzofuran-5-ylmethyl)amide
2-(1H-indazol-3-yl)-1H-benzimidazole-5-carboxylic acid 2-(methylsulphanyl)benzylamide
2-(1H-indazol-3-yl)-1H-benzimidazole-5-carboxylic acid (benzo[b]thiophen-3-ylmethyl)amide
2-(1H-indazol-3-yl)-1H-benzimidazole-5-carboxylic acid 3-methylbenzylamide
2-(1H-indazol-3-yl)-1H-benzimidazole-5-carboxylic acid 3-chlorobenzylamide
2-(1H-indazol-3-yl)-3H-benzimidazole-4-carboxylic acid 2-(methylsulphanyl)benzylamide

The subject of the present invention is also the process for preparing the products of formula (I) as defined above, characterized in that an acid of formula (D):

R1′-COOH (D)

in which R1′ has the meaning given above for R1, in which the possible reactive functions are optionally protected with protecting groups,
is subjected to an esterification reaction to give an acid ester of formula (II)

R1′-COOalk (II)

in which R1′ has the meaning given above and alk represents an alkyl radical,
is subjected to a reduction reaction to give the alcohol of formula (III):

R1′-CH2OH (III)

in which R1′ has the meaning given above,
which is oxidized to the aldehyde of formula (IV):

R1′-CHO (IV)

in which R1′ has the meaning given above,
and the products of formula (D) or products of formula (IV) as defined above are reacted with a diamine of formula (V):

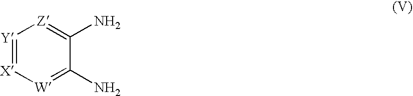

in which W′, X′, Y′ and Z′ have the meanings given above, respectively, for W, X, Y and Z, in which the possible reactive functions are optionally protected with protecting groups,
to give a product of formula (I′):

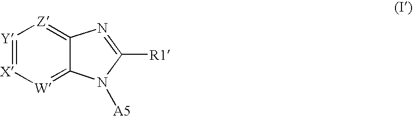

in which A5′ has the meaning as defined above for A5, in which the possible reactive functions are optionally protected with protecting groups, and R1′, W′, X′, Y′ and Z′ have the meanings given above,
the products of formula (I′) being products which may be products of formula (I) and which, in order to obtain products or other products of formula (I), may be subjected, if desired and if necessary, to one or more of the following conversion reactions, in any order:
a) an esterification reaction of an acid function,
b) a saponification reaction of an ester function to an acid function,
c) an oxidation reaction of an alkylthio group to the corresponding sulphoxide or sulphone,
d) a reaction for conversion of a ketone function to an oxime function,
e) a reaction for reduction of the free or esterified carboxyl function to an alcohol function,
f) a reaction for conversion of the alkoxy function to a hydroxyl function, or alternatively of the hydroxyl function to an alkoxy function,
g) a reaction for oxidation of an alcohol function to an aldehyde, acid or ketone function,
h) a reaction for conversion of a nitrile radical to a tetrazolyl,
i) a reaction for removal of the protecting groups that may be borne on the protected reactive functions,
j) a salification reaction with a mineral or organic acid or with a base to give the corresponding salt,
k) a reaction for resolution of the racemic forms into resolved products,
the said products of formula (I) thus being obtained in any possible racemic, enantiomeric or diastereoisomeric isomer form.

A subject of the present invention is, more particularly, the process for preparing the products of formula (I) as defined above, corresponding to formula (IA), characterized in that an acid of formula (D):

A′-COOH (D)

in which A′ has the meaning given above for A, in which the possible reactive functions are optionally protected with protecting groups,
is subjected to an esterification reaction to give an acid ester of formula (II)

A′-COOalk (II)

in which A′ has the meaning given above and alk represents an alkyl radical,
is subjected to a reduction reaction to give the alcohol of formula (III):

A′-CH2OH (III)

in which A′ has the meaning given above,
which is oxidized to the aldehyde of formula (IV):

A′-CHO (IV)

in which A′ has the meaning given above,
and the products of formula (D) or products of formula (IV) as defined above are reacted with a diamine of formula (V):

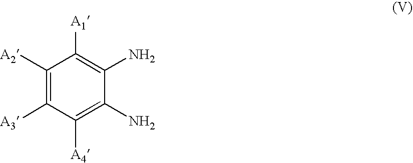

in which A1′, A2′, A3′ and A4′ have the meanings given above, respectively, for A1, A2, A3 and A4, in which the possible reactive functions are optionally protected with protecting groups, to give a product of formula (IA′):

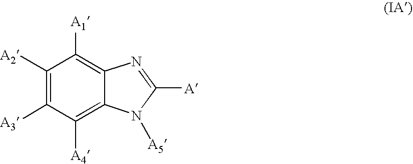

in which A5′ has the meaning as defined above A5, in which the possible reactive functions are optionally protected with protecting groups, and A1′, A2′, A3′ and A4′ have the meanings given above,
the products of formula (IA′) are products which may be products of formula (IA) and which, in order to obtain products or other products of formula (IA), may be subjected, if desired and if necessary, in any order, to one or more of the conversion reactions a) to k) as defined above,
the said products of formula (IA) thus obtained being in any possible racemic, enantiomeric or diastereoisomeric isomer form.

It may be noted that such conversion reactions of substituents into other substituents may also be carried out on the starting materials, and also on the intermediates as defined above before continuing the synthesis according to the reactions indicated in the process described above.

Under preferred conditions for carrying out the invention, the process described above may be performed as indicated in the schemes below: the reactions may be performed according to the usual conditions known to those skilled in the art and, for example, according to the reaction conditions indicated below.

Among the products of formula (I) of the present invention, certain products for which R1 represents a pyrazolyl or indazolyl radical may be obtained according to the following scheme from acid precursors:

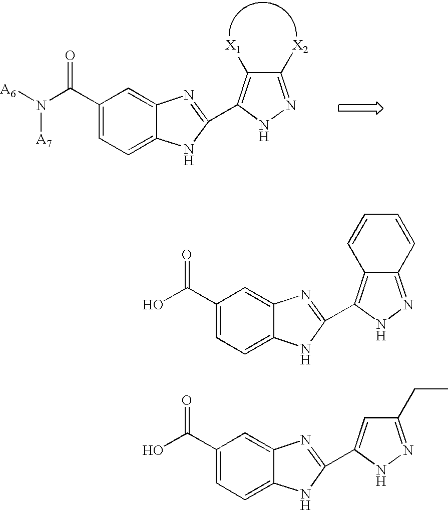

The following schemes indicate preferred routes for synthesizing the products of formula (I) of the invention:

I) Benzimidazole-indazole series, i.e. products of formula (I) for which R1 represents indazolyl:

### 1st Stage: Formation of the Aldehyde of Formula (IV):

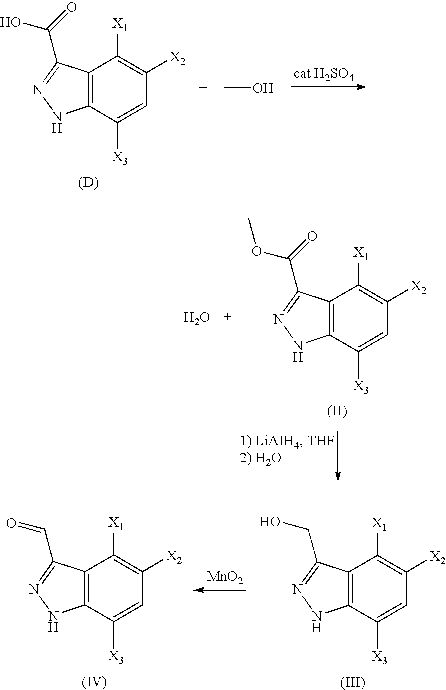

### 2^{nd} Stage: Formation of the Product of Formula (I):

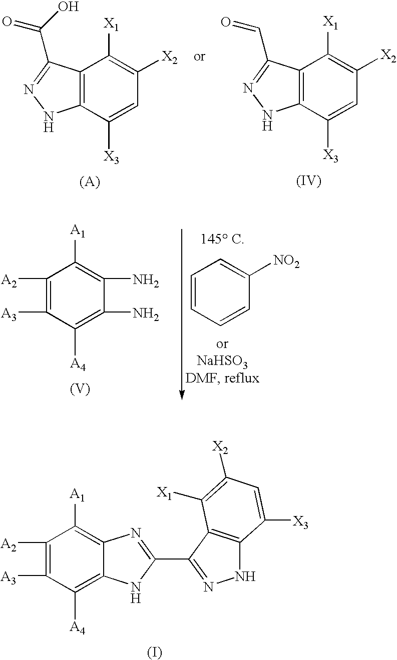

It may be noted that, when A2 or A3 or alternatively A1 or A4 represent a carboxyl radical, then A2 or A3, or A1 or A4 respectively, may be converted into an amide by the standard methods known to those skilled in the art, especially according to the standard methods of peptide coupling as indicated below.

In these products, the substituents A1, A2, A3, A4, A6, A7, X1, X2 and X3 have the meanings given above.

I) Benzimidazole-pyrazole series, i.e. products of formula (I) for which R1 represents pyrazolyl:

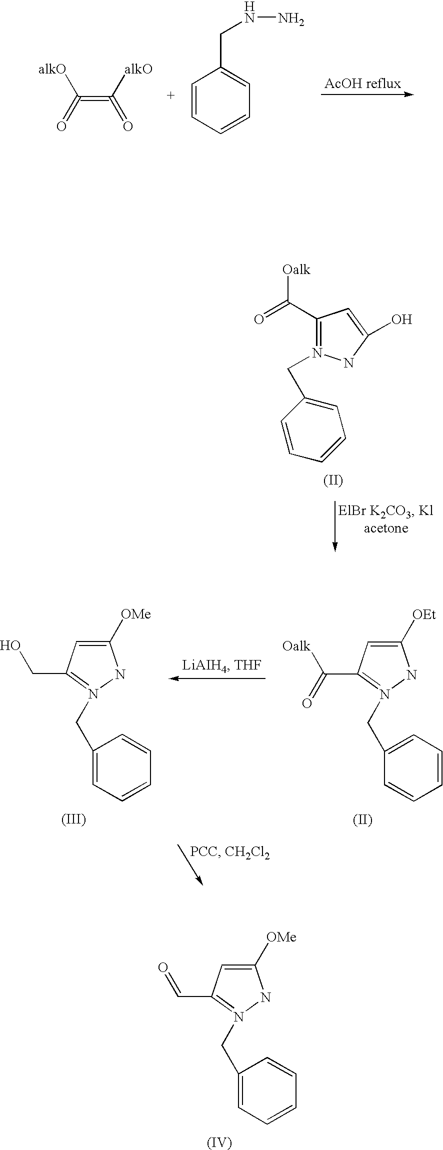

More generally, there is the following synthesis scheme:

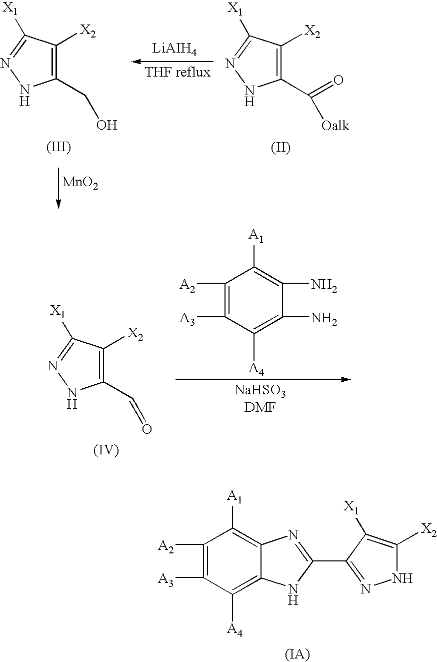

In the products of formula (IA) obtained, X1 may especially represent H and X2 optionally substituted thienyl.

In these products, for example, A1 and A4 may represent H and A3 and A4 may represent alkyl.

In the above products, the substituents A1, A2, A3, A4, A5, A6, A5, X1 and X2 have the meanings given above.

In the above schemes, the process may be performed in the same way by replacing

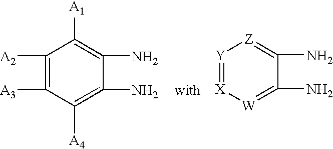

and the corresponding products are thus obtained.

As non-limiting examples illustrating the implementation of the process of the present invention, the synthesis of 4 products of formula (I) of the present invention may be represented by the following schemes:

Use may also be made of the synthesis route below for the preparation of the products of the present invention.

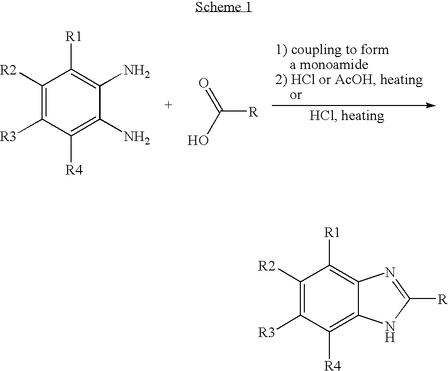

Conversion of the intermediates of the type A to a product of the type B by methods known to a person skilled in the art

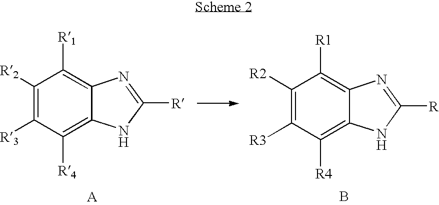

The substituents R1′ to R′4 and R′ are not necessarily only protecting groups but can also be functionalities which make possible the introduction of new substituents.

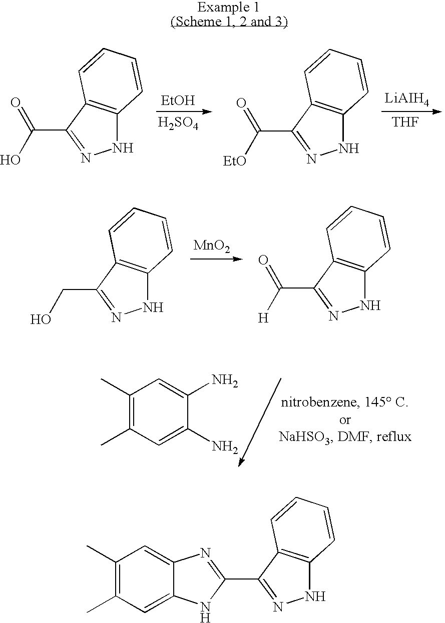

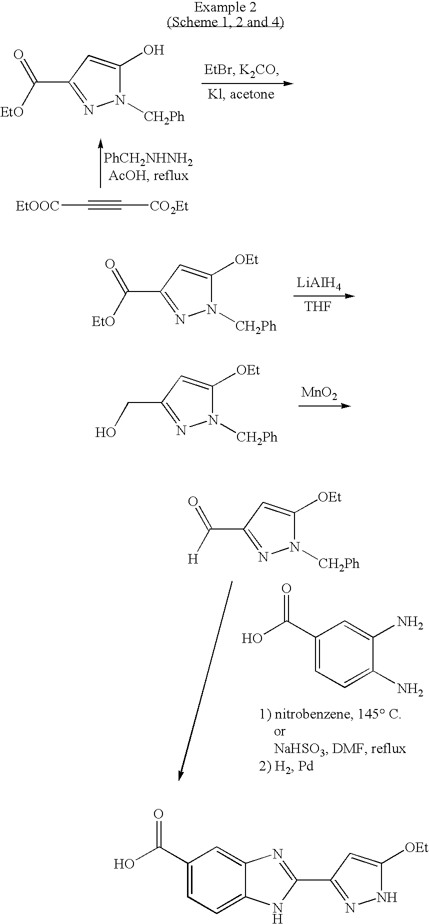

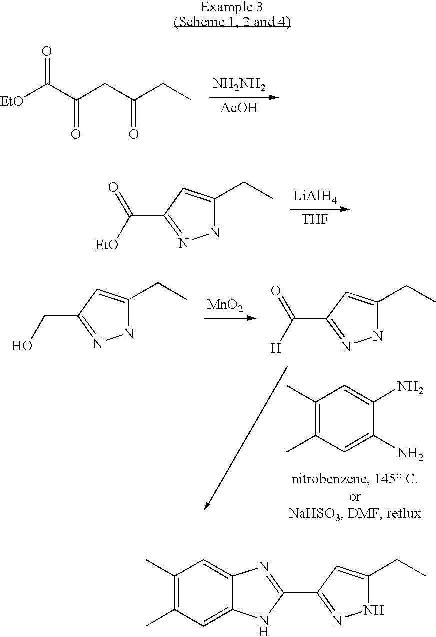

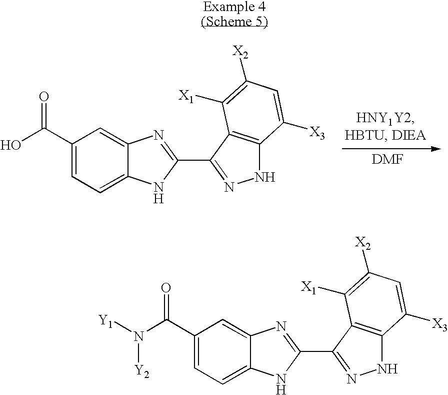

The acid esters constituted by the products of formula (II) may be obtained, if necessary, from the corresponding acids according to the usual methods and especially as indicated above.

Such acids may be commercially available, such as, for example, 3-carboxylndazole.

In the product of formula (II), the radical A′ especially represents a pyrazolyl or indazolyl radical.

The reaction for oxidation of the alcohols of formula (III) to the corresponding aldehydes of formula (IV) may be performed according to the usual techniques, for example using manganese perdioxide or chromium PCC salts of Swern type.

The aldehydes of formula (IV) thus obtained are reacted with a diamine of formula (V), especially in a solvent such as refluxing DMF in the presence of NaHSO₃

Among the diamines of formula (V), mention may be made, for example, of ortho-dianiline optionally substituted with one or more substituents chosen from the values of A1, A2, A3 and A4.

If necessary, the pyrazolyl radical may be formed as indicated in the above scheme, especially by reacting an alkyl acetylenedicarboxylate, for example methyl acetylenedicarboxylate, with a hydrazine.

Among the starting materials of formulae (II) and (V), some are known and may be obtained commercially or may be prepared according to the usual methods known to those skilled in the art.

Certain starting materials may also especially be prepared from commercial products, for example by subjecting them to one or more of the reactions described above in a) to k), performed under the reaction conditions that are also described above.

The experimental section below gives examples of such starting materials.

The following references are also cited, which may be used for the preparation of benzimidazoles, pyrazoles or indazoles in the context of the present invention:

G. R. Newkome, W. W. Paudler, Contemporary Heterocyclic Chemistry, Syntheses, Reactions and Applications, J. Wiley, 1982

Preston, Heterocyclic Compounds, Benzimidazoles and congeneric tricyclic compounds, J. Wiley, 1981

Behr, Fusco, Jarboe, Heterocyclic Compounds, Pyrazoles, Pyrazolines, Pyrazolidines, indazoles and condensed rings, J. Wiley, 1967.

According to the values of R1′, W′, X′, Y′, A′, A1′, A2′, A3′, A4′ and A5′, the products of formula (I′) or (IA′) may or may not constitute products of formula (I) or (IA) and may give products of formula (I) or (IA), or may be converted into other products of formula (I) or (IA) by being subjected to one or more of the reactions a) to k) indicated above.

Thus, the various reactive functions which may be borne by some of the compounds in the reactions defined above may, if necessary, be protected: these are, for example, hydroxyl, acyl, free carboxyl or amino and monoalkylamino radicals, which may be protected with suitable protecting groups.

The following non-exhaustive list of examples of protection of reactive functions may be cited:

the hydroxyl groups may be protected, for example, with alkyl radicals such as tert-butyl, trimethylsilyl, tert-butyldimethylsilyl, methoxymethyl, tetrahydropyranyl, benzyl or acetyl,

the amino groups may be protected, for example, with acetyl, trityl, benzyl, tert-butoxycarbonyl, BOC, benzyloxycarbonyl or phthalimido radicals or other radicals known in peptide chemistry,

the acyl groups such as the formyl group may be protected, for example, in the form of cyclic or acyclic ketals or thioketals, such as dimethyl or diethyl ketal or ethylenedioxy ketal, or diethylthio ketal or ethylenedithio ketal,

the acid functions in the products described above may, if desired, be amidated with a primary or secondary amine, for example in methylene chloride in the presence, for example, of 1-ethyl-3-(dimethylaminopropyl)carbodiimide hydrochloride at room temperature;

the acid functions may be protected, for example, in the form of esters formed with readily cleavable esters such as benzyl or tert-butyl esters or esters known in peptide chemistry.

The reactions a) to k) may be performed, for example, as indicated below.

a) The products described above may, if desired, be subjected, on the possible carboxyl functions, to esterification reactions which may be performed according to the usual methods known to those skilled in the art.

b) The possible conversions of ester functions into acid functions in the products described above may, if desired, be performed under the usual conditions known to those skilled in the art, especially by acid or alkaline hydrolysis, for example with sodium hydroxide or potassium hydroxide in alcoholic medium such as, for example, in methanol, or alternatively with hydrochloric acid or sulphuric acid.

The saponification reaction may be performed according to the usual methods known to those skilled in the art, such as, for example, in a solvent such as methanol or ethanol, dioxane or dimethoxyethane, in the presence of sodium hydroxide or potassium hydroxide.

c) The possible alkylthio groups in the products described above may, if desired, be converted into the corresponding sulphoxide or sulphone functions under the usual conditions known to those skilled in the art, such as, for example, with peracids such as, for example, peracetic acid or meta-chloroperbenzoic acid or alternatively with ozone, oxone or sodium periodate in a solvent such as, for example, methylene chloride or dioxane at room temperature.

The production of the sulphoxide function may be promoted by an equimolar mixture of the product containing an alkylthio group and of a reagent such as, especially, a peracid.

The production of the sulphone function may be promoted by a mixture of the product containing an alkylthio group with an excess of a reagent such as, especially, a peracid.

d) The reaction for the conversion of a ketone function to an oxime may be performed under the usual conditions known to those skilled in the art, such as, especially, an action in the presence of an optionally O-substituted hydroxylamine in an alcohol such as, for example, ethanol, at room temperature or with heating.

e) The possible free or esterified carboxyl functions in the products described above may, if desired, be reduced to an alcohol function by the methods known to those skilled in the art: the possible esterified carboxyl functions may, if desired, be reduced to an alcohol function by the methods known to those skilled in the art and especially with lithium aluminium hydride in a solvent such as, for example, tetrahydrofuran or dioxane or ethyl ether.

The possible free carboxyl functions in the products described above may, if desired, be reduced to an alcohol function especially with boron hydride (borane).

f) The possible alkoxy functions such as, especially, methoxy in the products described above may, if desired, be converted into a hydroxyl function under the usual conditions known to those skilled in the art, for example with boron tribromide in a solvent such as, for example, methylene chloride, with pyridine hydrobromide or hydrochloride or alternatively with hydrobromic acid or hydrochloric acid in water or trifluoroacetic acid at reflux.

g) The possible alcohol functions in the products described above may, if desired, be converted into an aldehyde or acid function by oxidation under the usual conditions known to those skilled in the art, such as, for example, by the action of manganese oxide to give aldehydes, or Jones reagent to give acids.

h) The possible nitrile functions in the products described above, may, if desired, be converted into tetrazolyl under the usual conditions known to those skilled in the art, such as, for example, by cycloaddition of a metal azide such as, for example, sodium azide or a trialkyltin azide with the nitrile function, as indicated in the method described in the article referenced as follows:

### J. Organometallic Chemistry., 33, 337 (1971) KOZIMA S. et al.

It may be noted that the reaction for the conversion of a carbamate to a urea and especially of a sulphonylcarbamate to a sulphonylurea may be performed, for example, in a refluxing solvent such as, for example, toluene in the presence of a suitable amine.

It is understood that the reactions described above may be carried out as indicated or alternatively, where appropriate, according to other usual methods known to those skilled in the art.

i) The removal of protecting groups such as, for example, those indicated above may be carried out under the usual conditions known to those skilled in the art, especially by an acid hydrolysis performed with an acid such as hydrochloric acid, benzenesulphonic acid or paratoluenesulphonic acid, formic acid or trifluoroacetic acid, or alternatively by a catalytic hydrogenation.

The phthalimido group may be removed with hydrazine.

A list of the various protecting groups that may be used will be found, for example, in patent BF 2 499 995.

j) The products described above may, if desired, be subjected to salification reactions, for example with a mineral or organic acid or with a mineral or organic base according to the usual methods known to those skilled in the art: such a salification reaction may be performed, for example, in the presence of hydrochloric acid, for example, or alternatively tartaric acid, citric acid or methanesulphonic acid, in an alcohol such as, for example, ethanol or methanol.

k) The possible optically active forms of the products described above may be prepared by resolution of the racemic mixtures according to the usual methods known to those skilled in the art.

Illustrations of such reactions defined above are given in the preparation of the examples described below.

The products of formula (I) as defined above and also the addition salts thereof with acids have advantageous pharmacological properties, especially on account of their kinase-inhibiting properties as indicated above.

It may be indicated that since certain kinase proteins have a central role in the initiation, development and completion of events of the cell cycle, molecules that inhibit such kinases are capable of limiting unwanted cell proliferations such as those observed in cancers, and can intervene in the prevention, regulation or treatment of neurodegenerative diseases such as Alzheimer's disease or neuronal apoptosis.

The products of the present invention are most particularly useful for preventing, regulating or treating diseases requiring anti-angiogenic activity.

The products of the present invention are especially useful for tumour therapy.

The products of the invention can thus also increase the therapeutic effects of commonly-used anti-tumoral agents. The products of formula (I) of the present invention thus most particularly have anti-angiogenic properties.

These properties justify their therapeutic use, and the subject of the invention is, particularly, as medicinal products, the products of formula (I) as defined above, the said products of formula (I) being in any possible racemic, enantiomeric or diastereoisomeric isomer form, and also the addition salts with pharmaceutically acceptable mineral and organic acids or with pharmaceutically acceptable mineral and organic bases of the said products of formula (I).

One subject of the invention is thus, more particularly, as medicinal products, the products as defined by formula (IA), (IAa) or (IAb), the said products of formula (IA), (IAa) or (IAb) being in any possible racemic, enantiomeric or diastereoisomeric isomer form, and also the addition salts with pharmaceutically acceptable mineral and organic acids or with pharmaceutically acceptable mineral and organic bases of the said products of formula (IA), (IAa) or (IAb).

One subject of the invention is, most particularly, as medicinal products, the products described below in the examples and especially the products corresponding to the following formulae:
2-(1H-indazol-3-yl)-1H-benzimidazole-5-carboxylic acid benzylamide
2-(1H-indazol-3-yl)-1H-benzimidazole-5-carboxylic acid N-methylamide
2-(1H-indazol-3-yl)-1H-benzimidazole-5-carboxylic acid N-ethylamide
2-(1H-indazol-3-yl)-1H-benzimidazole-5-carboxylic acid N-isopropylamide
2-(1H-indazol-3-yl)-1H-benzimidazole-5-carboxylic acid N-phenylamide
2-(1H-indazol-3-yl)-1H-benzimidazole-5-carboxylic acid N-phenethylamide
2-(1H-indazol-3-yl)-1H-benzimidazole-5-carboxylic acid N-morpholinoamide
2-(1H-indazol-3-yl)-1H-benzimidazole-5-carboxylic acid N—(N′-methyl-piperazino)amide
2-(1H-indazol-3-yl)-1H-benzimidazole-5-carboxylic acid N-pyrrolidinoamide
2-(1H-indazol-3-yl)-1H-benzimidazole-5-carboxylic acid N-(isobutyl)amide
2-(1H-indazol-3-yl)-1H-benzimidazole-5-carboxylic acid N-(cyclohexylmethyl)amide
2-(1H-indazol-3-yl)-1H-benzimidazole-5-carboxylic acid N-(2-furfuryl)amide
2-(1H-indazol-3-yl)-1H-benzimidazole-5-carboxylic acid N-benzyl-N-methylamide
methyl 2-(1H-indazol-3-yl)-3H-benzimidazole-5-carboxylate
5,6-dimethyl-2-(1H-indazol-3-yl)-1H-benzimidazole
5-methoxy-2-(1H-indazol-3-yl)-1H-benzimidazole
2-(1H-indazol-3-yl)-3H-benzimidazole-4-carboxylic acid
5-bromo 2-(1H-indazol-3-yl)-3H-benzimidazole
2-(5-ethoxy-2H-pyrazol-3-yl)-1H-benzimidazole-4-carboxylic acid
5,6-dimethyl-2-(5-methyl-2H-pyrazol-3-yl)-1H-benzimidazole
5,6-dimethyl-2-(5-thiophen-2-yl-2H-pyrazol-3-yl)-1H-benzimidazole
2-(4-bromo-2H-pyrazol-3-yl)-5,6-dimethyl-1H-benzimidazole
2-(5-ethyl-2H-pyrazol-3-yl)-5,6-dimethyl-1H-benzimidazole
2-(5-ethyl-2H-pyrazol-3-yl)-4,5-ethylenedioxy-1H-benzimidazole
2-(5-ethyl-2H-pyrazol-3-yl)-5-methoxy-1H-benzimidazole
2-(5-ethyl-2H-pyrazol-3-yl)-4-hydroxy-1H-benzimidazole
2-(5-ethyl-2H-pyrazol-3-yl)-5-bromo-1H-benzimidazole
2-(1H-indazol-3-yl)-1H-benzimidazole-5-carboxylic acid 4-(aminosulphonyl)benzylamide
2-(1H-indazol-3-yl)-1H-benzimidazole-5-carboxylic acid 4-bromobenzylamide
2-(1H-indazol-3-yl)-1H-benzimidazole-5-carboxylic acid 4-(methanesulphonyl)benzylamide
2-(1H-indazol-3-yl)-1H-benzimidazole-5-carboxylic acid 4-nitrobenzylamide
2-(1H-indazol-3-yl)-1H-benzimidazole-5-carboxylic acid 2-methylbenzylamide
2-(1H-indazol-3-yl)-1H-benzimidazole-5-carboxylic acid (6-chloropyridin-3-ylmethyl)amide
2-(1H-indazol-3-yl)-1H-benzimidazole-5-carboxylic acid (2,3-dihydrobenzofuran-5-ylmethyl)amide
2-(1H-indazol-3-yl)-1H-benzimidazole-5-carboxylic acid 2-(methylsulphanyl)benzylamide
2-(1H-indazol-3-yl)-1H-benzimidazole-5-carboxylic acid (benzo[b]thiophen-3-ylmethyl)amide
2-(1H-indazol-3-yl)-1H-benzimidazole-5-carboxylic acid 3-methylbenzylamide
2-(1H-indazol-3-yl)-1H-benzimidazole-5-carboxylic acid 3-chlorobenzylamide
2-(1H-indazol-3-yl)-3H-benzimidazole-4-carboxylic acid 2-(methylsulphanyl)benzylamide

One subject of the present invention is, most particularly, as medicinal products, the products of formula (I) as defined above, corresponding to the following formulae:
2-(1H-indazol-3-yl)-1H-benzimidazole-5-carboxylic acid benzylamide
2-(1H-indazol-3-yl)-1H-benzimidazole-5-carboxylic acid N-methylamide
2-(1H-indazol-3-yl)-1H-benzimidazole-5-carboxylic acid N-ethylamide
2-(1H-indazol-3-yl)-1H-benzimidazole-5-carboxylic acid N-isopropylamide
2-(1H-indazol-3-yl)-1H-benzimidazole-5-carboxylic acid N-phenylamide
2-(1H-indazol-3-yl)-1H-benzimidazole-5-carboxylic acid N-phenethylamide
2-(1H-indazol-3-yl)-1H-benzimidazole-5-carboxylic acid N-morpholinoamide
2-(1H-indazol-3-yl)-1H-benzimidazole-5-carboxylic acid N—(N′-methyl-piperazino)amide
2-(1H-indazol-3-yl)-1H-benzimidazole-5-carboxylic acid N-pyrrolidinoamide
2-(1H-indazol-3-yl)-1H-benzimidazole-5-carboxylic acid N-(isobutyl)amide
2-(1H-indazol-3-yl)-1H-benzimidazole-5-carboxylic acid N-(cyclohexylmethyl)amide
2-(1H-indazol-3-yl)-1H-benzimidazole-5-carboxylic acid N-(2-furfuryl)amide
2-(1H-indazol-3-yl)-1H-benzimidazole-5-carboxylic acid N-benzyl-N-methylamide
5-methoxy-2-(1H-indazol-3-yl)-1H-benzimidazole
2-(5-ethyl-2H-pyrazol-3-yl)-5,6-dimethyl-1H-benzimidazole
2-(5-ethyl-2H-pyrazol-3-yl)-4,5-ethylenedioxy-1H-benzimidazole
2-(5-ethyl-2H-pyrazol-3-yl)-5-methoxy-1H-benzimidazole
2-(5-ethyl-2H-pyrazol-3-yl)-4-hydroxy-1H-benzimidazole 2-(5-ethyl-2H-pyrazol-3-yl)-5-bromo-1H-benzimidazole.

One subject of the present invention is, most particularly, as medicinal products, the products of formula (I) as defined above, corresponding to the following formulae:
2-(1H-indazol-3-yl)-1H-benzimidazole-5-carboxylic acid 4-(aminosulphonyl)benzylamide
2-(1H-indazol-3-yl)-1H-benzimidazole-5-carboxylic acid 4-bromobenzylamide
2-(1H-indazol-3-yl)-1H-benzimidazole-5-carboxylic acid 4-(methanesulphonyl)benzylamide
2-(1H-indazol-3-yl)-1H-benzimidazole-5-carboxylic acid 4-nitrobenzylamide
2-(1H-indazol-3-yl)-1H-benzimidazole-5-carboxylic acid 2-methylbenzylamide
2-(1H-indazol-3-yl)-1H-benzimidazole-5-carboxylic acid (6-chloropyridin-3-ylmethyl)amide
2-(1H-indazol-3-yl)-1H-benzimidazole-5-carboxylic acid (2,3-dihydrobenzofuran-5-ylmethyl)amide
2-(1H-indazol-3-yl)-1H-benzimidazole-5-carboxylic acid 2-(methylsulphanyl)benzylamide
2-(1H-indazol-3-yl)-1H-benzimidazole-5-carboxylic acid (benzo[b]thiophen-3-ylmethyl)amide
2-(1H-indazol-3-yl)-1H-benzimidazole-5-carboxylic acid 3-methylbenzylamide
2-(1H-indazol-3-yl)-1H-benzimidazole-5-carboxylic acid 3-chlorobenzylamide
2-(1H-indazol-3-yl)-3H-benzimidazole-4-carboxylic acid 2-(methylsulphanyl)benzylamide

The invention also relates to pharmaceutical compositions containing, as active principle, at least one of the products of formula (I) as defined above, or a pharmaceutically acceptable salt of this product or a prodrug of this product and, where appropriate, a pharmaceutically acceptable support.

The invention thus covers pharmaceutical compositions containing, as active principle, at least one of the medicinal products as defined above.

Such pharmaceutical compositions of the present invention can also, where appropriate, contain active principles of other antimitotic medicinal products such as, in particular, those based on taxol, cis-platin, DNA-intercalating agents and the like.

These pharmaceutical compositions may be administered orally, parenterally or locally by topical application to the skin and mucous membranes or by intravenous or intramuscular injection.

These compositions may be solid or liquid and may be in any pharmaceutical form commonly used in human medicine, such as, for example, simple or sugar-coated tablets, pills, lozenges, gel capsules, drops, granules, injectable preparations, ointments, creams or gels; they are prepared according to the usual methods. The active principle may be incorporated therein with excipients usually used in these pharmaceutical compositions, such as talc, gum arabic, lactose, starch, magnesium stearate, cocoa butter, aqueous or non-aqueous vehicles, fatty substances of animal or plant origin, paraffin derivatives, glycols, and various wetting agents, dispersants, emulsifiers or preserving agents.

The usual dosage, which is variable depending on the product used, the individual treated and the complaint under consideration, may be, for example, from 0.05 to 5 g per day in adults, or preferably from 0.1 to 2 g per day.

The subject of the present invention is also the use of the products of formula (I) as defined above, or of pharmaceutically acceptable salts of these products, for the preparation of a medicinal product intended for inhibiting the activity of a kinase protein.

A subject of the present invention is also the use of products of formula (I) as defined above for the preparation of a medicinal product for treating or preventing a disease characterized by deregulation of the activity of a kinase protein.

Such a medicinal product may especially be intended for treating or preventing a disease in a mammal.

A subject of the present invention is also the use defined above, in which the kinase protein is a tyrosine kinase protein.

A subject of the present invention is also the use defined above, in which the kinase protein is chosen from the following group: FGFR1, FGFR2, FGFR3, FGFR4, FGFR5, flt-1, IGF-1R, KDR, PDGFR, tie2 and VEGFR.

A subject of the present invention is also the use defined above, in which the kinase protein is KDR.

A subject of the present invention is also the use defined above, in which the kinase protein is tie2.

A subject of the present invention is also the use defined above, in which the kinase protein is in a cell culture.

A subject of the present invention is also the use defined above, in which the kinase protein is in a mammal.

A subject of the present invention is particularly the use of a product of formula (I) as defined above, for the preparation of a medicinal product for treating or preventing a disease chosen from the following group: disorders of the proliferation of blood vessels, fibrotic disorders, disorders of the proliferation of “mesangial” cells, metabolic disorders, allergies, asthma, thrombosis, diseases of the nervous system, retinopathy, psoriasis, rheumatoid arthritis, diabetes, muscle degeneration and cancers.

A subject of the present invention is, more particularly, the use of a product of formula (I) as defined above, for the preparation of a medicinal product for treating or preventing a disease chosen from the following group: disorders of the proliferation of blood vessels, fibrotic disorders, disorders of the proliferation of “mesangial” cells, retinopathy, psoriasis, rheumatoid arthritis, diabetes, muscle degeneration and cancers.

A subject of the present invention is, most particularly, the use of a product of formula (I) as defined above, for the preparation of a medicinal product for preventing or treating diseases associated with an uncontrolled angiogenesis, for the preparation of a medicinal product for treating oncology diseases and especially intended for the treatment of cancers.

Among these cancers, the treatment of solid tumours and the treatment of cancers that are resistant to cytotoxic agents are of interest.

Among these cancers, the treatment of breast cancer, stomach cancer, cancer of the ovaries, cancer of the colon, lung cancer, brain cancer, cancer of the larynx, cancer of the lymphatic system, cancer of the genitourinary tract including the bladder and the prostate, bone cancer and cancer of the pancreas, and most particularly treatment of breast cancer, cancer of the colon or lung cancer, are of interest.

A subject of the present invention is also the use of the products of formula (I) as defined above, for the preparation of medicinal products for cancer chemotherapy.

Such medicinal products intended for cancer chemotherapy may be used alone or in combination.

The products of the present invention may especially be administered alone or in combination with chemotherapy or radiotherapy or alternatively in combination, for example, with other therapeutic agents.

Such therapeutic agents may be commonly-used anti-tumoral agents.

As kinase inhibitors, mention may be made of butyrolactone, flavopiridol and 2-(2-hydroxyethylamino)-6-benzylamino-9-methylpurine, also known as olomucine.

A subject of the present invention is also the products of formula (I) as defined above as KDR inhibitors.

A subject of the present invention is also the products of formula (I) as defined above as tie2 inhibitors.

The products described in the 3 tables below headed Tables I, II and III form part of the present invention and these products, and also the products described in the experimental section, are products of formula (I) which illustrate the invention without, however, limiting it.

TABLE I

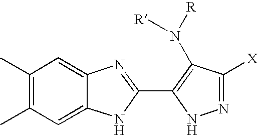

with X represents hydrogen, halogen or alkoxy as defined above.

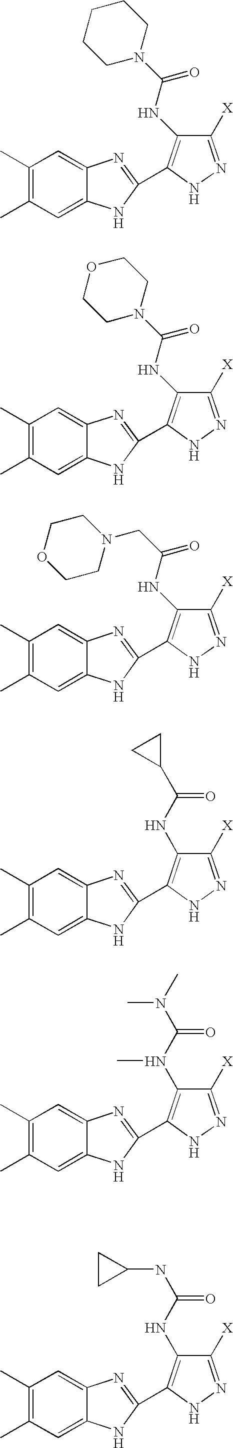

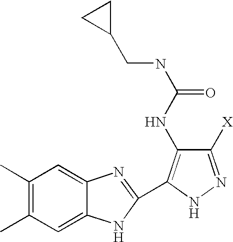

TABLE II

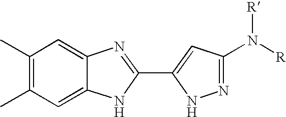

in which NR′R represents NY1Y2 as defined above.

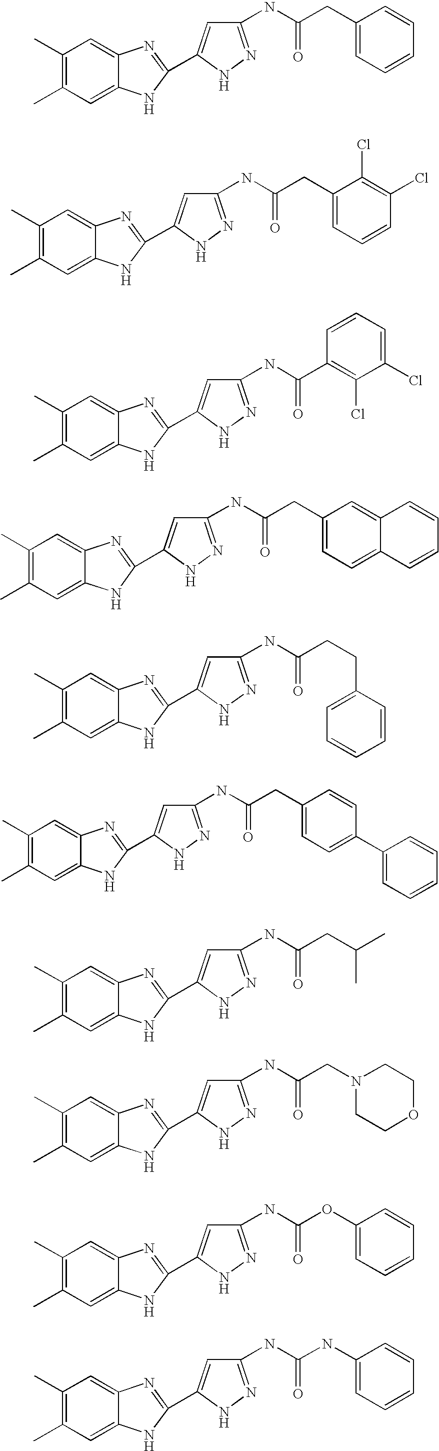

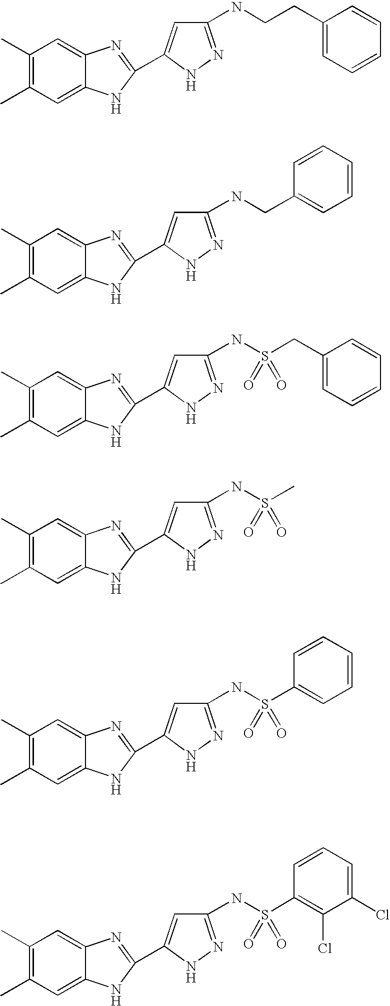

TABLE III

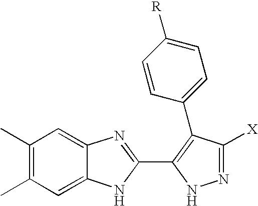

in which X represents hydrogen, alkynyl or NHCOCH2Ph which is optionally substituted.

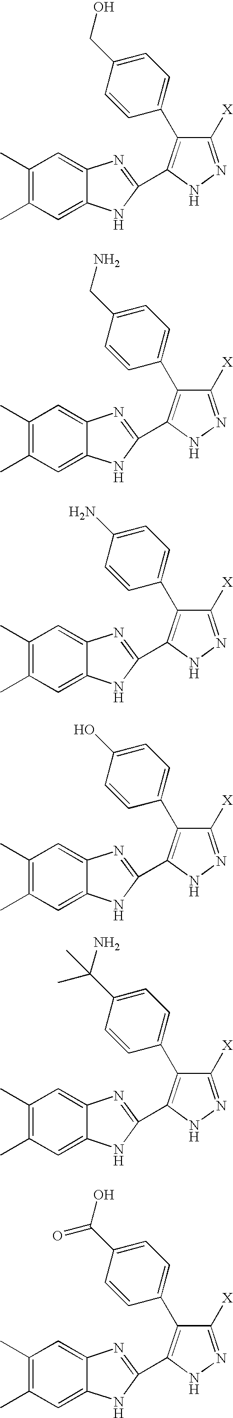

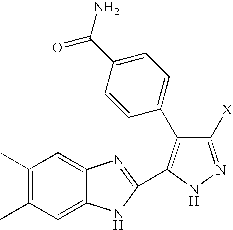

### EXAMPLES

### General Method of LC/MS Purification

A Waters FractionLynx system is used, and the separations were carried out on a Waters Symmetry column (C18, 5 μM, 19×50 mm, catalogue number 186000210), eluting with a linear gradient of acetonitrile containing 0.07% TFA (v/v) in water containing 0.07% TFA (v/v), gradient rising from 5% to 95% (v/v) of acetonitrile/TFA over 8 minutes, and then 2 minutes at 95% acetonitrile/TFA at a flow rate of 10 ml/min. The products are injected in solution in DMSO, and collected according to the detection of their molecular weight.

The chemical shifts (6) in the NMR descriptions are given in ppm.

### Example 1

### 2-(1H-Indazol-3-yl)-1H-benzimidazole-5-carboxylic acid benzylamide

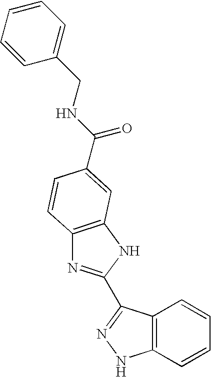

2-(1H-Indazol-3-yl)-1H-benzimidazole-5-carboxylic acid benzylamide may be prepared in the following manner.

A solution of 27.3 mg of HBTU (O-benzotriazol-1-yl-N,N,N′,N′-tetramethyluronium hexafluorophosphate) in 0.2 ml of dimethylformamide is added, at a temperature of about 20° C., to a solution of 20 mg of 2-(1H-indazol-3-yl)-1H-benzimidazole-5-carboxylic acid in 0.42 ml of anhydrous dimethylformamide. After stirring at a temperature of about 20° C. for one hour, 15.7 ml of benzylamine is added, followed by addition of 12.4 ml of N,N-diisopropylethylamine dissolved in 0.32 ml of dimethylformamide. After 20 hours, at a temperature of about 20° C., the reaction medium is concentrated under reduced pressure, at a temperature of about 40° C. The crude residue obtained is dissolved in DMSO and purified by preparative LC/MS. The fractions containing the desired product are combined and concentrated under reduced pressure at a temperature of about 40° C. to afford 20 mg of 2-(1H-indazol-3-yl)-1H-benzimidazole-5-carboxylic acid benzylamide are thus obtained in the form of a cream-coloured powder, the characteristics of which are as follows:

LC/MS retention time=2.86 minutes

2-(1H-Indazol-3-yl)-1H-benzimidazole-5-carboxylic acid may be prepared in the following manner:

1.3 g of sodium metabisulphite and 1.04 g of 3,4-diaminobenzoic acid are added, at a temperature of about 20° C., to a solution of 1 g of 1H-indazole-3-carboxaldehyde in 10 ml of dimethylformamide. The reaction mixture is refluxed for one hour, then cooled to a temperature of about 20° C. and diluted with dichloromethane, and the mixture is filtered. The collected filtrate is concentrated under reduced pressure. The brown lacquer obtained (340 mg) is purified by preparative LC/MS. 138.8 mg of 5,6-dimethyl-2-(1H-indazol-3-yl)-1H-benzimidazole are thus obtained in the form of a beige-coloured powder.

1H-Indazole-3-carboxaldehyde may be prepared in the following manner:

A solution of 2.27 g of (1H-indazol-3-yl)methanol in 220 ml of 1,2-dimethoxyethane is added to 13.32 g of manganese dioxide. After one hour at a temperature of about 20° C., the reaction mixture is refluxed for 15 minutes. After cooling to a temperature of about 20° C., the reaction medium is filtered through a sinter funnel packed with Celite® (diatomaceous earth) (Celite Corporation, 137 West Central Avenue, Lompor, Calif. 93436). The collected filtrate is concentrated under reduced pressure at a temperature of about 40° C. 2.02 g of 1H-indazole-3-carboxaldehyde are thus obtained in the form of a yellow powder, the characteristics of which are as follows:

1H NMR, DMSO d6, 400 MHz: 7.40 ppm (triplet, 1H); 7.55 ppm (triplet, 1H); 7.75 ppm (doublet, 1H); 8.18 ppm (doublet, 1H); 10.23 ppm (singlet, 1H); 14.2 ppm (multiplet, 1H).

(1H-Indazol-3-yl)methanol may be prepared in the following manner:

3.2 g of lithium aluminium hydride are added portionwise to a solution of 7.08 g of methyl 3-indazolecarboxylate in 80 ml of tetrahydrofuran, cooled to a temperature of about 0° C. by an ice bath. After 4 hours at a temperature of about 0° C., 1.6 g of lithium aluminium hydride are added. After 2 hours at a temperature of about 0° C., the reaction medium is treated successively with 6 ml of water and then 6 ml of aqueous 1N sodium hydroxide solution and finally 18 ml of water. The reaction mixture is filtered through paper and the aqueous filtrate is then extracted with dichloromethane. The collected organic fractions are combined, dried over magnesium sulphate and concentrated under reduced pressure at a temperature of about 40° C. 3.15 g of (1H-indazol-3-yl)methanol are obtained in the form of an off-white powder, the characteristics of which are as follows:

¹H NMR, DMSO d6, 400 MHz: 4.80 ppm (doublet, 2H); 5.25 ppm (triplet, 1H); 7.15 ppm (triplet, 1H); 7.35 ppm (triplet, 1H); 7.51 ppm (doublet, 1H); 7.87 ppm (doublet, 1H); 12.81 ppm (multiplet, 1H).

Methyl 3-indazolecarboxylate may be prepared in the following manner:

0.5 ml of concentrated sulphuric acid (95%) is added dropwise, at a temperature of about 20° C., to a solution of 9.13 g of 3-indazolecarboxylic acid in 100 ml of methanol. After refluxing for 20 hours, the reaction medium is concentrated under reduced pressure at a temperature of about 40° C. The aqueous residue obtained is extracted with dichloromethane. The organic phases are combined, washed with water until neutral, dried over magnesium sulphate and then concentrated under reduced pressure at a temperature of about 40° C. The yellow powder obtained is washed with ethyl ether. A white powder is obtained. The filtrate is concentrated under reduced pressure until a yellow powder is obtained. This yellow powder is washed again with ethyl ether until a white powder is obtained. The yellow filtrate is concentrated a third time under reduced pressure and the yellow powder collected is itself also washed with ethyl ether. All the fractions of white powder are combined. 7.08 g of methyl 3-indazolecarboxylate are thus obtained in the form of a white powder.

### Example 2

### 2-(1H-Indazol-3-yl)-1H-benzimidazole-5-carboxylic acid N-methylamide

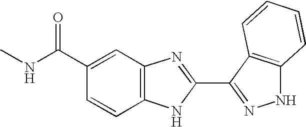

2-(1H-Indazol-3-yl)-1H-benzimidazole-5-carboxylic acid N-methylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 71.8 μl of a methylamine solution (2M in tetrahydrofuran), 14.8 mg of expected product are obtained.

### Example 3

### 2-(1H-Indazol-3-yl)-1H-benzimidazole-5-carboxylic acid N-ethylamide

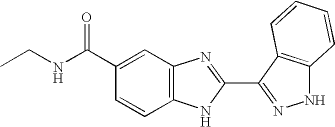

2-(1H-Indazol-3-yl)-1H-benzimidazole-5-carboxylic acid N-ethylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 19.4 ml of an ethylamine solution (33% in water), 14.8 mg of 2-(1H-indazol-3-yl)-1H-benzimidazole-5-carboxylic acid N-ethylamide are obtained.

### Example 4

### 2-(1H-Indazol-3-yl)-1H-benzimidazole-5-carboxylic acid N-isopropylamide

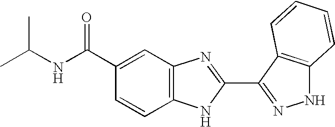

2-(1H-Indazol-3-yl)-1H-benzimidazole-5-carboxylic acid N-isopropylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 12.3 ml of isopropylamine, 16.5 mg of 2-(1H-indazol-3-yl)-1H-benzimidazole-5-carboxylic acid N-isopropylamide are obtained.

### Example 5

### 2-(1H-Indazol-3-yl)-1H-benzimidazole-5-carboxylic acid N-phenylamide

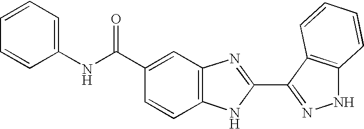

2-(1H-Indazol-3-yl)-1H-benzimidazole-5-carboxylic acid N-phenylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 13.1 ml of aniline, 14.1 mg of 2-(1H-indazol-3-yl)-1H-benzimidazole-5-carboxylic acid N-phenylamide are obtained in the form of a white powder.

### Example 6

### 2-(1H-Indazol-3-yl)-1H-benzimidazole-5-carboxylic acid N-phenethylamide

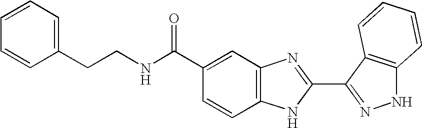

2-(1H-Indazol-3-yl)-1H-benzimidazole-5-carboxylic acid N-phenethylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 18 ml of phenethylamine, 17.7 mg of 2-(1H-indazol-3-yl)-1H-benzimidazole-5-carboxylic acid N-phenethylamide are obtained in the form of a white powder.

### Example 7

### 2-(1H-Indazol-3-yl)-1H-benzimidazole-5-carboxylic acid N-morpholinoamide

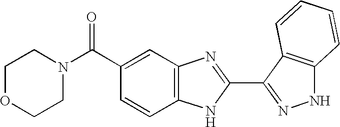

2-(1H-Indazol-3-yl)-1H-benzimidazole-5-carboxylic acid N-morpholinoamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 12.5 ml of morpholine, 18.6 mg of 2-(1H-indazol-3-yl)-1H-benzimidazole-5-carboxylic acid N-morpholinoamide are obtained in the form of a pale yellow powder.

### Example 8

### 2-(1H-Indazol-3-yl)-1H-benzimidazole-5-carboxylic acid N—(N′-methyl-piperazino)amide

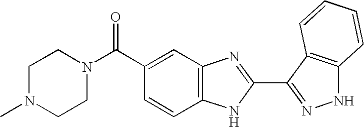

2-(1H-Indazol-3-yl)-1H-benzimidazole-5-carboxylic acid N—(N′-methyl-piperazino)amide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 15.9 ml of N-methylpiperazine, 16.1 mg of 2-(1H-indazol-3-yl)-1H-benzimidazole-5-carboxylic acid N—(N′-methyl-piperazino)amide are obtained in the form of a yellow oil.

### Example 9

### 2-(1H-Indazol-3-yl)-1H-benzimidazole-5-carboxylic acid N-pyrrolidinoamide

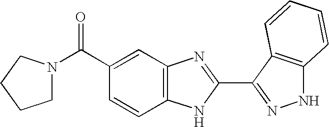

2-(1H-Indazol-3-yl)-1H-benzimidazole-5-carboxylic acid N-pyrrolidinoamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 12 ml of pyrrolidine, 17.7 mg of 2-(1H-indazol-3-yl)-1H-benzimidazole-5-carboxylic acid N-pyrrolidinoamide are obtained in the form of a pale yellow powder.

### Example 10

### 2-(1H-Indazol-3-yl)-1H-benzimidazole-5-carboxylic acid N-(isobutyl)amide

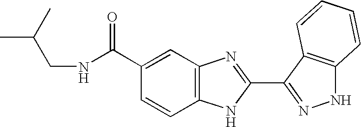

2-(1H-Indazol-3-yl)-1H-benzimidazole-5-carboxylic acid N-(isobutyl)amide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 14.6 ml of isobutylamine, 7.6 mg of 2-(1H-indazol-3-yl)-1H-benzimidazole-5-carboxylic acid N-(isobutyl)amide are obtained in the form of a pale yellow powder.

### Example 11

### 2-(1H-Indazol-3-yl)-1H-benzimidazole-5-carboxylic acid N-(cyclohexylmethyl)amide

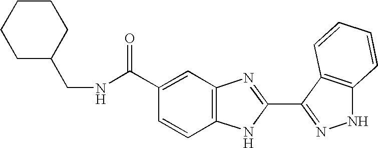

2-(1H-Indazol-3-yl)-1H-benzimidazole-5-carboxylic acid N-(cyclohexylmethyl)amide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 18.7 ml of cyclohexylmethylamine, 16.1 mg of 2-(1H-indazol-3-yl)-1H-benzimidazole-5-carboxylic acid N-(cyclohexylmethyl)amide are obtained in the form of a white powder.

### Example 12

### 2-(1H-Indazol-3-yl)-1H-benzimidazole-5-carboxylic acid N-(2-furfuryl)amide

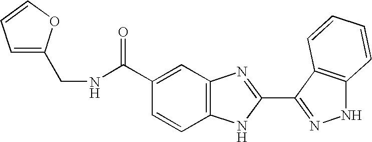

2-(1H-Indazol-3-yl)-1H-benzimidazole-5-carboxylic acid N-(2-furfuryl)amide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 13.3 ml of 2-furfurylamine, 14.8 mg of 2-(1H-indazol-3-yl)-1H-benzimidazole-5-carboxylic acid N-(2-furfuryl)amide are obtained in the form of a white powder.

### Example 13

### 2-(1H-Indazol-3-yl)-1H-benzimidazole-5-carboxylic acid N-benzyl-N-methylamide

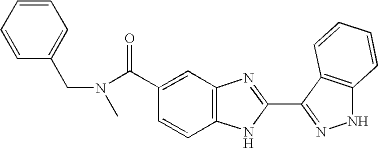

2-(1H-Indazol-3-yl)-1H-benzimidazole-5-carboxylic acid N-benzyl-N-methylamide may be prepared by following the procedure for the preparation of 2-(1H-indazol-3-yl)-1H-benzimidazole-5-carboxylic acid N-benzylamide (Example 1):

Starting with 20 mg of 2-(1H-indazol-3-yl)-1H-benzimidazole-5-carboxylic acid and 18.6 ml of N-methylbenzylamine, 7.3 mg of 2-(1H-indazol-3-yl)-1H-benzimidazole-5-carboxylic acid N-benzyl-N-methylamide are obtained in the form of a pale yellow powder.

### Example 14

### Methyl 2-(1H-indazol-3-yl)-3H-benzimidazole-5-carboxylate

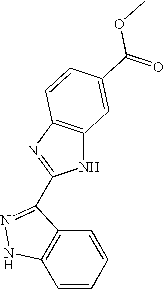

Methyl 2-(1H-indazol-3-yl)-3H-benzimidazole-5-carboxylate may be prepared in the following manner:

A mixture of 0.1 g of 1H-indazole-3-carboxaldehyde and 113.7 mg of methyl 3,4-diaminobenzoate in 10 ml of nitrobenzene is maintained at a temperature of about 145° C. for 3 hours and 45 minutes. After cooling to a temperature of about 20° C., the reaction mixture is purified on SPE (5 g of SCX phase, processing and washing with methanol, extraction with a 2N ammoniacal methanol solution). The ammoniacal solution collected during the detachment is then concentrated under reduced pressure at a temperature of about 40° C. 198.3 mg of an orange lacquer is obtained and purified by preparative LC/MS. 42.7 mg of methyl 2-(1H-indazol-3-yl)-3H-benzimidazole-5-carboxylate are thus obtained in the form of a beige-coloured powder, the characteristics of which are as follows:

1H NMR, DMSO d6, 400 MHz: 3.95 ppm (singlet, 3H); 7.40 ppm (triplet, 1H); 7.55 ppm (triplet, 1H); 7.75 ppm (doublet, 1H); 7.77 ppm (doublet, 1H); 7.95 ppm (doublet, 1H); 8.57 ppm (doublet, 1H); 13.85 ppm (multiplet, 1H).

### Example 15

### 5,6-Dimethyl-2-(1H-indazol-3-yl)-1H-benzimidazole

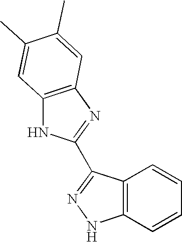

5,6-Dimethyl-2-(1H-indazol-3-yl)-1H-benzimidazole may be prepared by following the procedure for the preparation of methyl 2-(1H-indazol-3-yl)-3H-benzimidazole-5-carboxylate (Example 14):

Starting with 200 mg of 1H-indazole-3-carboxaldehyde and 177 mg of 4,5-dimethyl-1,2-phenylenediamine in 10 ml of nitrobenzene, 15.9 mg of 5,6-dimethyl-2-(1H-indazol-3-yl)-1H-benzimidazole are obtained in the form of a dark red powder, the characteristics of which are as follows:

1H NMR, DMSO d6, 400 MHz: 2.60 ppm (singlet, 6H); 7.42 ppm (triplet, 1H); 7.53 ppm (singlet, 2H); 7.58 ppm (triplet, 1H); 7.78 ppm (doublet, 1H); 8.52 ppm (doublet, 1H); 14.05 ppm (multiplet, 1H).

5,6-Dimethyl-2-(1H-indazol-3-yl)-1H-benzimidazole may also be prepared according to the following procedure:

389 mg of sodium metabisulphite are added, at a temperature of about 20° C., to a solution of 300 mg of 1H-indazole-3-carboxaldehyde and 279 mg of 4,5-dimethyl-1,2-phenylenediamine in 3 ml of dimethylformamide. The reaction mixture is refluxed for 4 hours and then cooled to a temperature of about 20° C. and filtered through paper. The collected filtrate is concentrated under reduced pressure. The brown lacquer obtained (340 mg) is purified by preparative LC/MS. 138.8 mg of 5,6-dimethyl-2-(1H-indazol-3-yl)-1H-benzimidazole are thus obtained in the form of a beige-coloured powder.

### Example 16

### 5-Methoxy-2-(1H-indazol-3-yl)-1H-benzimidazole

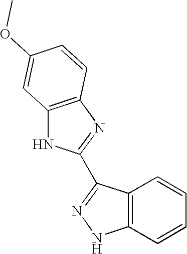

5-Methoxy-2-(1H-indazol-3-yl)-1H-benzimidazole may be prepared by following the procedure for the preparation of methyl 2-(1H-indazol-3-yl)-3H-benzimidazole-5-carboxylate (Example 14):

Starting with 200 mg of 1H-indazole-3-carboxaldehyde and 274.4 mg of 4-methoxy-1,2-phenylenediamine dihydrochloride in 10 ml of nitrobenzene, 45.6 mg of 5-methoxy-2-(1H-indazol-3-yl)-1H-benzimidazole are obtained in the form of a light brown powder, the characteristics of which are as follows:

1H NMR, DMSO d6, 400 MHz: 3.90 ppm (singlet, 3H); 7.00 ppm (doublet, 1H); 7.18 ppm (doublet, 1H); 7.40 ppm (triplet, 1H); 7.55 ppm (triplet, 1H); 7.64 ppm (doublet, 1H); 7.73 ppm (doublet, 1H); 8.52 ppm (doublet, 1H); 13.91 ppm (multiplet, 1H).

### Example 17

### 2-(1H-Indazol-3-yl)-3H-benzimidazole-4-carboxylic acid

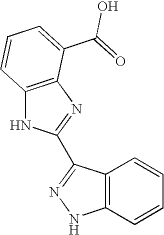

2-(1H-Indazol-3-yl)-3H-benzimidazole-4-carboxylic acid may be prepared by following the procedure for the preparation of methyl 2-(1H-indazol-3-yl)-3H-benzimidazole-5-carboxylate (Example 14):

Starting with 237 mg of 1H-indazole-3-carboxaldehyde and 305.5 mg of 2,3-diaminobenzoic acid hydrochloride in 10 ml of nitrobenzene, 20.5 mg of 2-(1H-indazol-3-yl)-3H-benzimidazole-4-carboxylic acid 5-methoxyamide of 2-(1H-indazol-3-yl)-1H-benzimidazole acid are obtained in the form of a beige-coloured powder, the characteristics of which are as follows:

1H NMR, DMSO d6, 400 MHz: 7.40 ppm (triplet, 1H); 7.42 ppm (triplet, 1H); 7.55 ppm (triplet, 1H); 7.72 ppm (doublet, 1H); 7.90 ppm (doublet, 1H); 8.02 ppm (doublet, 1H); 8.52 ppm (doublet, 1H); 13.68 ppm (multiplet, 1H).

### Example 18

### 5-Bromo-2-(1H-indazol-3-yl)-3H-benzimidazole

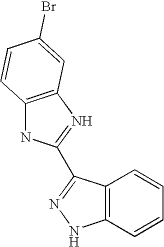

5-Bromo-2-(1H-indazol-3-yl)-3H-benzimidazole may be pre-pared by following the procedure for the preparation of 5,6-dimethyl-2-(1H-indazol-3-yl)-1H-benzimidazole (Example 15):

Starting with 643 mg of 1H-indazole-3-carboxaldehyde, 816 mg of 4-bromo-1,2-phenylenediamine, and 836.5 mg of sodium metabisulphite in 15 ml of dimethylformamide, and after purification by SPE (SCX phase, washing with methanol, extraction with 2N ammoniacal methanol) followed by a chromatography under pressure on silica, 939 mg of 5-bromo-2-(1H-indazol-3-yl)-3H-benzimidazole are obtained in the form of a brick-red powder.

### Example 19

### 2-(5-Ethoxy-2H-pyrazol-3-yl)-1H-benzimidazole-4-carboxylic acid

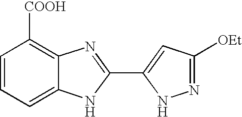

2-(5-Ethoxy-2H-pyrazol-3-yl)-1H-benzimidazole-4-carboxylic acid may be obtained from 2-(2-benzyl-5-ethoxy-2H-pyrazol-3-yl)-1H-benzimidazole-4-carboxylic acid by deprotection of the benzyl group in the presence of hydrogen and a catalyst such as palladium.

2-(2-Benzyl-5-ethoxy-2H-pyrazol-3-yl)-1H-benzimidazole-4-carboxylic acid may be prepared by following the procedure for the preparation of 5,6-dimethyl-2-(1H-indazol-3-yl)-1H-benzimidazole (Example 15):

Starting with 21.6 mg of 2-benzyl-5-ethoxy-2H-pyrazole-3-carboxaldehyde, and 17.7 mg of 2,3-diaminobenzoic acid hydrochloride in 1 ml of nitrobenzene, and after purification by SPE (SCX phase, washing with methanol, extraction with 2N ammoniacal methanol), 50.9 mg of 2-(2-benzyl-5-ethoxy-2H-pyrazol-3-yl)-1H-benzimidazole-4-carboxylic acid are obtained in the form of a yellow lacquer.

2-Benzyl-5-ethoxy-2H-pyrazole-3-carboxaldehyde may be prepared in the following manner:

4 Å molecular sieves are added to a solution of 45.7 mg of (2-benzyl-5-ethoxy-2H-pyrazol-3-yl)methanol in 0.5 ml of dichloromethane, followed by addition of 43.1 mg of pyridinium chlorochromate. After 20 hours at a temperature of about 20° C., the reaction mixture is filtered through Celite®. The insoluble material formed is rinsed with ethyl acetate and then with dichloromethane. The filtrate is washed with water. After separation of the phases by settling, the aqueous phase is re-extracted with dichloromethane. The organic phases are combined, dried over magnesium sulphate, filtered and then concentrated under reduced pressure. 21.6 mg of 2-benzyl-5-ethoxy-2H-pyrazole-3-carboxaldehyde are thus obtained in the form of a brown lacquer, the characteristics of which are as follows:

1H NMR, DMSO d6, 400 MHz: 1.35 ppm (triplet, 3H); 4.25 ppm (quartet, 2H); 5.30 ppm (singlet, 2H); 6.30 ppm (singlet, 1H); 7.25-7.40 ppm (multiplet, 5H); 9.72 ppm (singlet, 1H).

(2-Benzyl-5-ethoxy-2H-pyrazol-3-yl)methanol may be prepared in the following manner:

11.1 mg of lithium aluminium hydride are added to a solution of 76 mg of methyl 2-benzyl-5-ethoxy-2H-pyrazole-3-carboxylate in 0.75 ml of tetrahydrofuran, cooled to a temperature of about 0° C. by an ice bath. After 3 hours at a temperature of about 0° C., 22.2 mg of lithium aluminium hydride are added and the reaction medium is allowed to warm to a temperature of about 20° C. After 30 minutes at a temperature of about 20° C., 10 ml of ice-cold water are added and the reaction mixture is then filtered through Celite®. After separation of the phases by settling, the aqueous phase is extracted with ethyl acetate. The organic phases are combined, dried over magnesium sulphate and concentrated under reduced pressure. 45.7 mg of (2-benzyl-5-ethoxy-2H-pyrazol-3-yl)methanol are thus obtained in the form of a brown lacquer, the characteristics of which are as follows:

1H NMR, DMSO d6, 400 MHz: 1.35 ppm (triplet, 3H); 4.15 ppm (quartet, 2H); 4.30 ppm (doublet, 2H); 5.00 ppm (triplet, 1H); 5.08 ppm (singlet, 2H); 5.70 ppm (singlet, 1H); 7.20-7.40 ppm (multiplet, 5H).

Methyl 2-benzyl-5-ethoxy-2H-pyrazole-3-carboxylate may be prepared in the following manner:

5 mg of sodium iodide, 36 μl of bromoethane and 70 mg of potassium carbonate are added, at a temperature of about 20° C., to a solution of 100 mg of methyl 2-benzyl-5-hydroxy-2H-pyrazole-3-carboxylate in 1 ml of acetone. The reaction mixture is refluxed for 9 hours, cooled to a temperature of about 20° C. and filtered. The filtrate is concentrated under reduced pressure. 76 mg of methyl 2-benzyl-5-ethoxy-2H-pyrazole-3-carboxylate are thus obtained in the form of a solid, the characteristics of which are as follows:

1H NMR, DMSO d6, 400 MHz: 1.35 ppm (triplet, 3H); 3.50 ppm (singlet, 3H); 4.22 ppm (quartet, 2H); 5.22 ppm (singlet, 2H); 6.28 ppm (singlet, 1H); 7.20-7.40 ppm (multiplet, 5H).

Methyl 2-benzyl-5-hydroxy-2H-pyrazole-3-carboxylate may be prepared in the following manner:

1.72 ml of dimethylacetylene dicarboxylate are added, at a temperature of about 20° C., to a solution of 2.73 g of benzylhydrazine dihydrochloride in 45 ml of glacial acetic acid. The reaction mixture is refluxed for 3 hours, cooled to a temperature of about 20° C. and then concentrated under reduced pressure. After filtering off the insoluble material formed, 252 mg of methyl 2-benzyl-5-hydroxy-2H-pyrazole-3-carboxylate are collected in the form of a white powder, the characteristics of which are as follows:

1H NMR, DMSO d6, 400 MHz: 3.76 ppm (singlet, 3H); 5.19 ppm (singlet, 2H); 5.85 ppm (singlet, 1H); 7.25-7.45 ppm (multiplet, 5H); 11.69 ppm (multiplet, 1H).

The filtrate may be purified by flash chromatography on 400 g of 20-45 μm silica (applied in a 25/75 ethyl acetate/cyclohexane mixture; eluant: 25/75 and then 40/60 ethyl acetate/cyclohexane) to give an additional batch of methyl 2-benzyl-5-hydroxy-2H-pyrazole-3-carboxylate in the form of a white powder.

### Example 20

### 5,6-Dimethyl-2-(5-methyl-2H-pyrazol-3-yl)-1H-benzimidazole

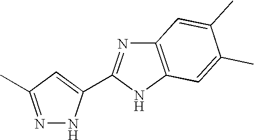

5,6-Dimethyl-2-(5-methyl-2H-pyrazol-3-yl)-1H-benzimidazole may be prepared by following the procedure described for the preparation of 5,6-dimethyl-2-(1H-indazol-3-yl)-1H-benzimidazole (Example 15):

Starting with 53.3 mg of 5-methyl-2H-pyrazole-3-carboxaldehyde, 65.9 mg of 4,5-dimethyl-1,2-phenylenediamine, and 92 mg of sodium metabisulphite, in 0.5 ml of ethanol and 1.5 ml of dimethylformamide, and after purification by SPE (SCX phase, washing with methanol, extraction with 2N ammoniacal methanol) followed by a chromatography under pressure on silica, 20.8 mg of 5,6-dimethyl-2-(5-methyl-2H-pyrazol-3-yl)-1H-benzimidazole are obtained in the form of a white powder.

5-Methyl-2H-pyrazole-3-carboxaldehyde may be prepared from commercial ethyl 5-methyl-2H-pyrazole-3-carboxylate by following the procedure described for the preparation of 1H-indazole-3-carboxaldehyde, starting with methyl 3-indazolecarboxylate.

### Example 21

### 5,6-Dimethyl-2-(5-thiophen-2-yl-2H-pyrazol-3-yl)-1H-benzimidazole

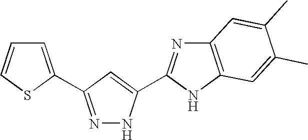

5,6-Dimethyl-2-(5-thiophen-2-yl-2H-pyrazol-3-yl)-1H-benzimidazole may be prepared by following the procedure described for the preparation of 5,6-dimethyl-2-(1H-indazol-3-yl)-1H-benzimidazole (Example 15):

Starting with 16.2 mg of 5-thiophen-2-yl-2H-pyrazole-3-carboxaldehyde, 12.4 mg of 4,5-dimethyl-1,2-phenylenediamine, and 17.3 mg of sodium metabisulphite, in 0.2 ml of ethanol and 0.6 ml of dimethylformamide, and after purification by SPE (SCX phase, washing with methanol, extraction with 2N ammoniacal methanol) followed by chromatography under pressure on silica and purification by LC/MS, 5,6-dimethyl-2-(5-thiophen-2-yl-2H-pyrazol-3-yl)-1H-benzimidazole is obtained in the form of a white powder.

5-Thiophen-2-yl-2H-pyrazole-3-carboxaldehyde may be prepared from commercial ethyl 5-thiophen-2-yl-2H-pyrazole-3-carboxylate by following the procedure described for the preparation of 1H-indazole-3-carboxaldehyde starting with methyl 3-indazolecarboxylate (Example 1).

### Example 22

### 2-(4-Bromo-2H-pyrazol-3-yl)-5,6-dimethyl-1H-benzimidazole

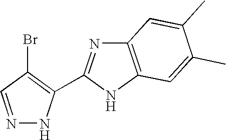

2-(4-Bromo-2H-pyrazol-3-yl)-5,6-dimethyl-1H-benzimidazole may be prepared by following the procedure described for the preparation of 5,6-dimethyl-2-(1H-indazol-3-yl)-1H-benzimidazole (Example 15):

Starting with 100 mg of commercial 4-bromo-2H-pyrazole-3-carboxaldehyde, 77.8 mg of 4,5-dimethyl-1,2-phenylenediamine, and 108.6 mg of sodium metabisulphite, in 1 ml of ethanol and 2 ml of dimethylformamide, and after purification by SPE (SCX phase, washing with methanol, extraction with 2N ammoniacal methanol) followed by chromatography under pressure on silica, 143.2 mg of 2-(4-bromo-2H-pyrazol-3-yl)-5,6-dimethyl-1H-benzimidazole are obtained in the form of a yellow foam.

### Example 23

### 2-(5-Ethyl-2H-pyrazol-3-yl)-5,6-dimethyl-1H-benzimidazole

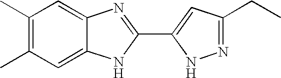

2-(5-Ethyl-2H-pyrazol-3-yl)-5,6-dimethyl-1H-benzimidazole may be prepared by following the procedure described for the preparation of 5,6-dimethyl-2-(1H-indazol-3-yl)-1H-benzimidazole (Example 15):

Starting with 100 mg of 5-ethyl-2H-pyrazole-3-carboxaldehyde, 110 mg of 4,5-dimethyl-1,2-phenylenediamine, and 153 mg of sodium metabisulphite, in 1 ml of ethanol and 3 ml of dimethylformamide, and after purification by SPE (SCX phase, washing with methanol, extraction with 2N ammoniacal methanol) followed by reverse-phase HPLC (5 mm C18 phase, dimensions 100×25 mm, flow rate 20 ml/min, elution gradient acetonitrile/0.07% TFA—water/0.07% TFA from 5-95 to 95-5 (v/v)), and desalification by SPE (SCX phase, washing with methanol, extraction with 2N ammoniacal methanol), 82 mg of 2-(5-ethyl-2H-pyrazol-3-yl)-5,6-dimethyl-1H-benzimidazole are obtained in the form of a beige-coloured powder, the characteristics of which are as follows:

1H NMR, DMSO d6, 300 MHz: 1.26 (t, J=7 Hz: 3H); 2.31 (s: 6H); 2.70 (broad q, J=7 Hz: 2H); 6.60 (broad s: 1H); 7.22 (mult: 1H); 7.36 (mult: 1H); 12.37 (mult: 1H); 12.92 (mult: 1H).

5-Ethyl-2H-pyrazole-3-carboxaldehyde may be prepared from ethyl 5-ethyl-2H-pyrazole-3-carboxylate by following the procedure described for the preparation of 1H-indazole-3-carboxaldehyde starting with methyl 3-indazolecarboxylate (Example 1).

Ethyl 5-ethyl-2H-pyrazole-3-carboxylate may be prepared according to the general procedure in the following reference: Kunio Seki et al., Chem. Pharm. Bull., 32(4), 1568-1577 (1984).

### Example 24

### 2-(5-Ethyl-2H-pyrazol-3-yl)-4,5-ethylenedioxy-1H-benzimidazole

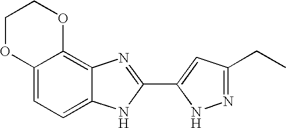

2-(5-Ethyl-2H-pyrazol-3-yl)-4,5-ethylenedioxy-1H-benzimidazole may be prepared by following the procedure described for the preparation of 5,6-dimethyl-2-(1H-indazol-3-yl)-1H-benzimidazole (Example 15):

Starting with 100 mg of 5-ethyl-2H-pyrazole-3-carboxaldehyde, 134 mg of 3,4-ethylenedioxy-1,2-phenylenediamine, and 153 mg of sodium metabisulphite, in 1 ml of ethanol and 3 ml of dimethylformamide, and after purification by SPE (SCX phase, washing with methanol, extraction with 2N ammoniacal methanol) followed by reverse-phase HPLC (5 mm, C18 phase, dimensions 100×25 mm, flow rate 20 ml/min, elution gradient acetonitrile/0.07% TFA—water/0.07% TFA from 5-95 to 95-5 (v/v)), and desalification by SPE (SCX phase, washing with methanol, extraction with 2N ammoniacal methanol), 60 mg of 2-(5-ethyl-2H-pyrazol-3-yl)-4,5-ethylenedioxy-1H-benzimidazole are obtained in the form of a brown lacquer, the characteristics of which are as follows:

1H NMR, DMSO d6, 300 MHz: 1.27 (t, J=7 Hz: 3H); 2.70 (broad q, J=7 Hz: 2H); from 4.20 to 4.45 (mt: 4H); 6.61 (broad s: 1H); 6.72 (d, J=8 Hz: 1H); 6.88 (broad d, J=8 Hz: 1H); 12.50 (mult: 1H); 12.94 (mult: 1H).

### Example 25

### 2-(5-Ethyl-2H-pyrazol-3-yl)-5-methoxy-1H-benzimidazole

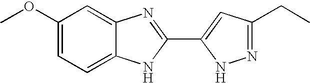

2-(5-Ethyl-2H-pyrazol-3-yl)-5-methoxy-1H-benzimidazole may be prepared by following the procedure described for the preparation of 5,6-dimethyl-2-(1H-indazol-3-yl)-1H-benzimidazole (Example 15):

Starting with 100 mg of 5-ethyl-2H-pyrazole-3-carboxaldehyde, 138 mg of 4-methoxy-1,2-phenylenediamine, and 153 mg of sodium metabisulphite, in 1 ml of ethanol and 3 ml of dimethylformamide, and after purification by SPE (SCX phase, washing with methanol, extraction with 2N ammoniacal methanol) followed by reverse-phase HPLC (5 mm C18 phase, dimensions 100×25 mm, flow rate 20 ml/min, elution gradient: acetonitrile/0.07% TFA—water/0.07% TFA from 5-95 to 95-5 (v/v)), and desalification by SPE (SCX phase, washing with methanol, extraction with 2N ammoniacal methanol), 61 mg of 2-(5-ethyl-2H-pyrazol-3-yl)-5-methoxy-1H-benzimidazole are obtained in the form of a brown lacquer, the characteristics of which are as follows:

1H NMR, DMSO d6 with addition of a few drops of CD3COOD, 300 MHz: 1.26 (t, J=7 Hz: 3H); 2.70 (q, J=7 Hz: 2H); 3.79 (s: 3H); 6.61 (s: 1H); 6.81 (dd, J=8.5 and 2.5 Hz: 1H); 7.03 (broad s: 1H); 7.42 (d, J=8.5 Hz: 1H).

### Example 26

### 2-(5-Ethyl-2H-pyrazol-3-yl)-4-hydroxy-1H-benzimidazole

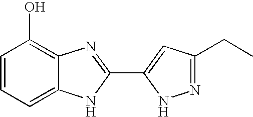

2-(5-Ethyl-2H-pyrazol-3-yl)-4-hydroxy-1H-benzimidazole may be prepared by following the procedure described for the preparation of 5,6-dimethyl-2-(1H-indazol-3-yl)-1H-benzimidazole (Example 15):

Starting with 100 mg of 5-ethyl-2H-pyrazole-3-carboxaldehyde, 100 mg of 2,3-diaminophenol, and 153 mg of sodium metabisuiphite, in 1 ml of ethanol and 3 ml of dimethylformamide, and after purification by SPE (SCX phase, washing with methanol, extraction with 2N ammoniacal methanol) followed by reverse-phase HPLC (5 mm, C18 phase, dimensions: 100×25 mm, flow rate 20 ml/min, elution gradient: acetonitrile/0.07% TFA—water/0.07% TFA from 5-95 to 95-5 (v/v)), and desalification by SPE (SCX phase, washing with methanol, extraction with 2N ammoniacal methanol), 16 mg of 2-(5-ethyl-2H-pyrazol-3-yl)-4-hydroxy-1H-benzimidazole are obtained in the form of a brown lacquer, the characteristics of which are as follows:

1H NMR, DMSO d6 with addition of a few drops of CD3COOD, 300 MHz: 1.26 (t, J=7 Hz: 3H); 2.70 (q, J=7 Hz: 2H); 6.55 (t, J=4.5 Hz: 1H); 6.66 (s: 1H); 6.96 (broad d, J=4.5 Hz: 2H).

### Example 27

### 2-(5-Ethyl-2H-pyrazol-3-yl)-5-bromo-1H-benzimidazole

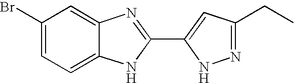

2-(5-Ethyl-2H-pyrazol-3-yl)-5-bromo-1H-benzimidazole may be prepared by following the procedure described for the preparation of 5,6-dimethyl-2-(1H-indazol-3-yl)-1H-benzimidazole (Example 15):

Starting with 20 mg of 5-ethyl-2H-pyrazole-3-carboxaldehyde, 30 mg of 4-bromo-1,2-phenylenediamine and 30 mg of sodium metabisulphite, in 1 ml of ethanol and 2 ml of dimethylformamide, and after purification by SPE (SCX phase, washing with methanol, extraction with 2N ammoniacal methanol) followed by reverse-phase HPLC (5 mm C18 phase, dimensions: 100×25 mm, flow rate 20 ml/min, elution gradient: acetonitrile/0.07% TFA—water/0.07% TFA from 5-95 to 95-5 (v/v)), and desalification by SPE (SCX phase, washing with methanol, extraction with 2N ammoniacal methanol), 21 mg of 2-(5-ethyl-2H-pyrazol-3-yl)-5-bromo-1H-benzimidazole are obtained in the form of a yellow powder, the characteristics of which are as follows:

1H NMR, DMSO d6, 300 MHz: 1.28 (t, J=7 Hz: 3H); 2.71 (q, J=7 Hz: 2H); 6.67 (s: 1H); 7.30 (dd, J=8.5 and 2.5 Hz: 1H); 7.49 (mt: 1H); 7.712 (broad s: 1H); from 12.5 to 13.5 (broad mult: 2H).

The products of formula (I) of the present invention can also be prepared according to the following process:

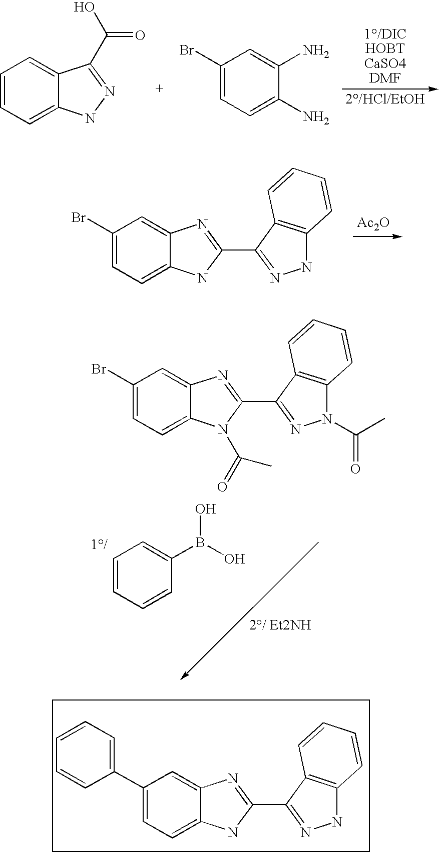

The products of Examples 97 to 145 of the present invention represented in the table IV and can be prepared according to the schemes indicated above and in particular according to the procedures indicated below.

### Example 97

### 3-(6-Phenyl-1H-benzimidazol-2-yl)-2H-indazole

### Step 1: Synthesis of 3-(6-bromo-1H-benzimidazol-2-yl)-2H-indazole

4.25 g of 1-hydroxybenzotriazole (HOBT) and 4.3 g of calcium sulphate are added at ambient temperature to a solution of 4.6 g of indazole-3-carboxylic acid in 50 ml of dimethylformamide. The reaction mixture is cooled to approximately 0° C. and then 4.9 ml of N,N-diisopropylcarbodiimide (DIC) are slowly added. After stirring for 2 hours at ambient temperature, 5.9 g of 4-bromo-o-phenylenediamine are added. After stirring for 60 hours at ambient temperature, the reaction mixture is concentrated to dryness under reduced pressure. The brown oil obtained is taken up in 50 ml of water and extracted 3 times with 50 ml of ethyl acetate. The organic phases are combined, dried over magnesium sulphate and then concentrated to dryness under reduced pressure. 18 g of a brown oil are thus obtained, which oil is taken up in 100 ml of a 20% solution of hydrochloric acid in ethanol. The mixture is brought to reflux for 4 hours and then concentrated to dryness, the brown oil obtained is taken up in 20 ml of water, and an aqueous ammonia solution is added until a pH of the mixture of about 8-9 is obtained. The aqueous phase is then extracted 3 times with 30 ml of ethyl acetate and the organic phases are combined, dried over magnesium sulphate and concentrated to dryness under reduced pressure. After purification by chromatography under pressure on silica (eluent water/acetonitrile), 5 g of 3-(6-bromo-1H-benzimidazol-2-yl)-2H-indazole are thus obtained.

IR spectrum (KBr): characteristic bands at 1621, 1570, 1441, 1344, 1324, 1273, 1239, 1135, 1042, 914, 804, 774 and 746 cm⁻¹

### Step 2: Synthesis of 1-[2-(1-acetyl-1H-indazol-3-yl)-5-benzimidazol-1-yl]ethanone

5 g of 3-(6-bromo-1H-benzimidazol-2-yl)-2H-indazole are charged to a solution of 40 ml of acetic anhydride and 40 ml of pyridine. The mixture is brought to reflux for 4 hours, then brought to ambient temperature and concentrated to dryness. The brown solid obtained is taken up in 50 ml of ethyl acetate and washed with 50 ml of a saturated sodium hydrogen carbonate solution until a pH of 7-8 is obtained. The organic phase is dried over magnesium sulphate, filtered and then concentrated to dryness under reduced pressure. The light brown solid obtained is triturated in 20 ml of ethyl acetate and then filtered off on a sintered glass funnel. 1.5 g of the compound I-[2-(1-acetyl-1H-indazol-3-yl)-5-bromobenzimidazol-1-yl]ethanone are thus obtained. A second crop is obtained by chromatographing the filtrate obtained above under pressure on silica (eluent cyclohexane/ethyl acetate), i.e. 1.3 g of the same compound.

### Characteristics of the Compound:

¹H NMR spectrum (300 MHz, (CD₃)2SO d6, 8 in ppm).

The mixture of the two positional isomers in the proportions 50/50 is observed.

2.61 and 2.62 (2 s, 3H in all); 2.80 (s, 3H); 7.62 (broad t, J=7.5 Hz, 1H); 7.68 and 7.71 (2 dd, J=9 and 2 Hz, 1H in all); 7.80 (ddd, J=8.5, 7.5 and 0.5 Hz, 1H); 7.91 and 8.01 (2 d, J=9 Hz, 1H); 8.18 and 8.20 (2 d, J=2 Hz, 1H in all); 8.27 and 8.30 (2 d, J=7.5 Hz, 1H in all); 8.46 (d, J=8.5 Hz, 1H)

IR spectrum (KBr): characteristic bands at 1727, 1610, 1450, 1405, 1374, 1326, 1290, 1198, 1176, 964 and 760 cm⁻¹

### Step 3: Synthesis of 3-(6-phenyl-1H-benzimidazol-2-yl)-2H-indazole

40 mg of sodium carbonate, 7 mg of dihydrogendichlorobis(di-tert-butylphosphonite-κP)palladate(2-) (POPd[0]) and 46 mg of phenylboronic acid are added under an argon atmosphere to a solution of 50 mg of 1-[2-(1-acetyl-1H-indazol-3-yl)-5-bromobenzimidazol-1-yl]ethanone in 800 μl of anhydrous tetrahydrofuran. The reaction mixture is brought to reflux for 3 hours and then cooled to ambient temperature. The mixture is then diluted with 3 ml of ethyl acetate and then washed with 2 times with 2 ml of water. The organic phase is dried over magnesium sulphate and then concentrated to dryness under reduced pressure. 48 mg of a brown solid are obtained, which solid is dissolved in 500 μl of tetrahydrofuran, to which 500 μl of diethylamine are added. The reaction mixture is heated at 60° C. for 4 hours and then allowed to return to ambient temperature. The mixture is then concentrated to dryness and then the brown solid obtained is purified by LC/MS to produce 12.5 mg of 3-(6-phenyl-1H-benzimidazol-2-yl)-2H-indazole (6); analytical retention time 3.10, MS 311 [M+H]⁺.

The products of formula (I) of the present invention and in particular the products of Examples 98 to 145 can also be prepared according to the following process:

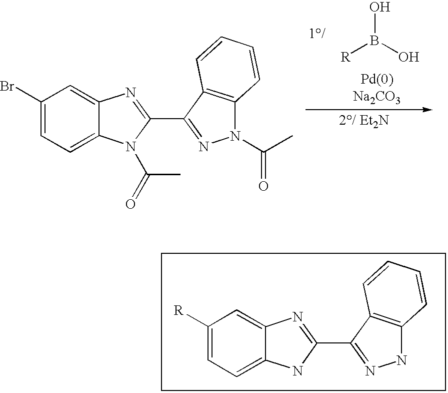

The synthesis of the products of Examples 98 to 145 can be carried out in a similar way to the synthesis of 3-(6-phenyl-1H-benzimidazol-2-yl)-2H-indazole (Example 97) but replacing phenylboronic acid by boronic acids of the formula RB(OH)₂.

The products of formula (I) of the present invention constituted by Examples 28 to 96 and 146 to 180 of the present invention are represented in table IV. These products can be prepared in particular according to the schemes indicated above and especially as indicated above for the product of Example 1.

TABLE IV

| | | | MS | |
|---|---|---|---|---|
| | | | Obs. | |
| | | | Ion, amu | |
| | | | (M + H)⁺ | MS |
| | Structure, | | (unless | Retn |
| Ex. | Name, | RNH₂ or | otherwise | Time |
| No. | Molecular Formula and MW | RB(OH)₂ | noted) | (min) |
| 28 | 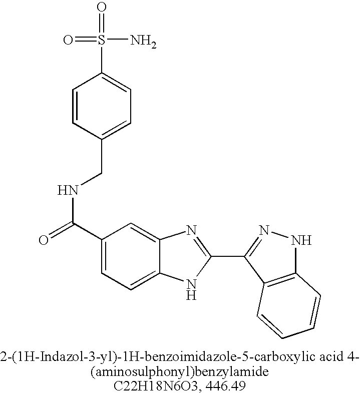 | 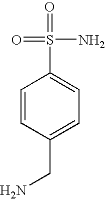 | 447 | 2.77 |
| 29 | 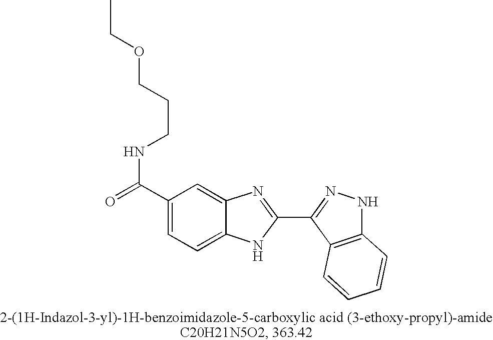 | 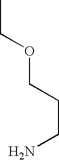 | 364 | 2.8 |
| 30 | 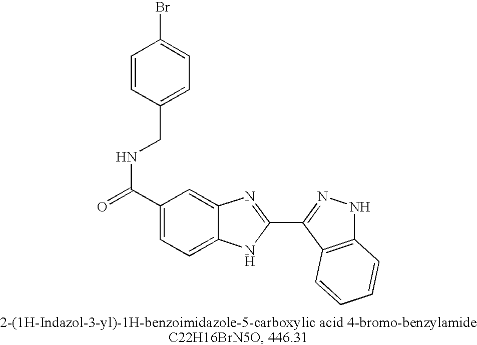 | 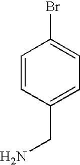 | 447 | 3.35 |
| 31 | 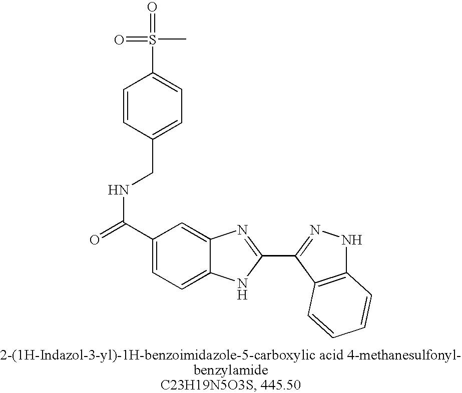 | 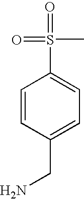 | 446 | 2.81 |
| 32 | 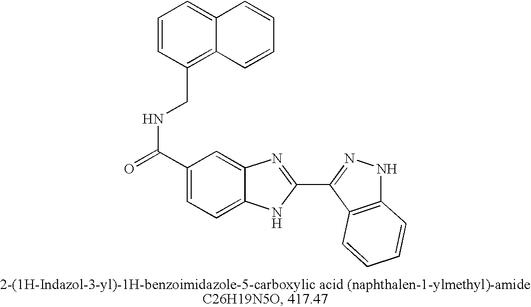 | 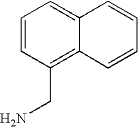 | 418 | 3.38 |
| 33 | 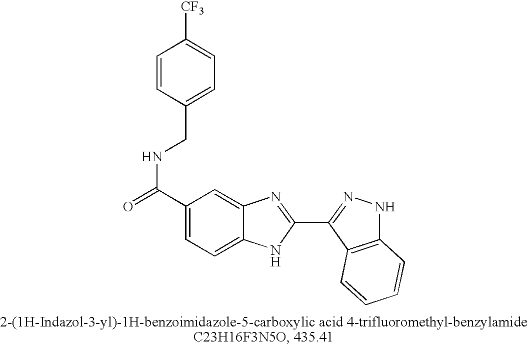 | 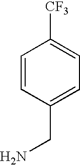 | 436 | 3.41 |
| 34 | 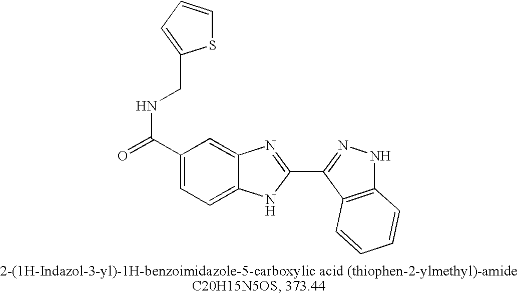 | 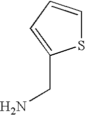 | 374 | 3.01 |
| 35 | 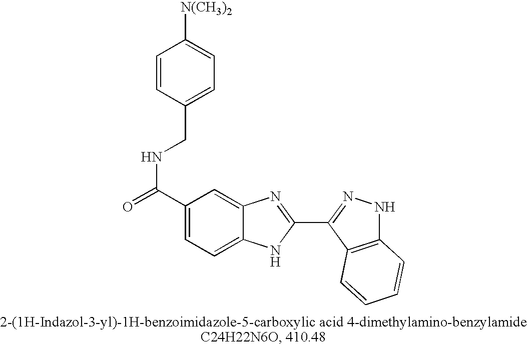 | 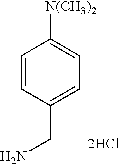 | 411 | 2.49 |
| 36 | 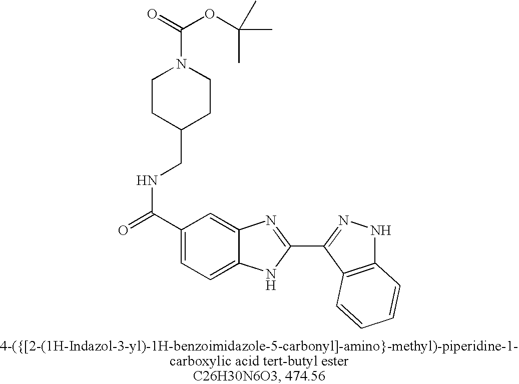 | 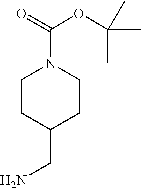 | 475 | 3.31 |
| 37 | 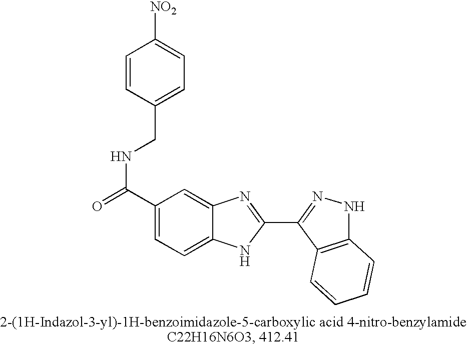 | 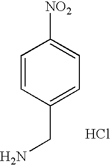 | 413 | 3.14 |
| 38 | 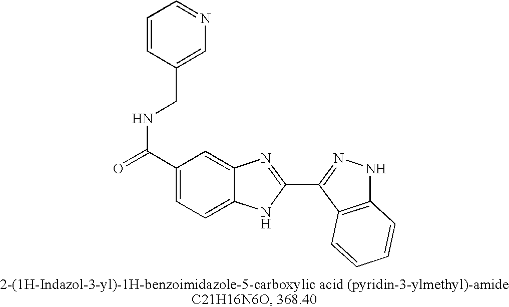 | 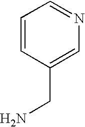 | 369 | 2.39 |
| 39 | 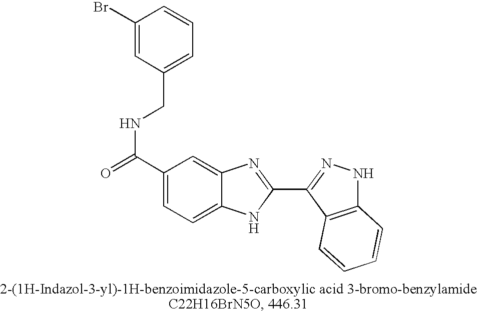 | 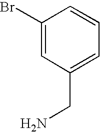 | 447 | 3.36 |
| 40 | 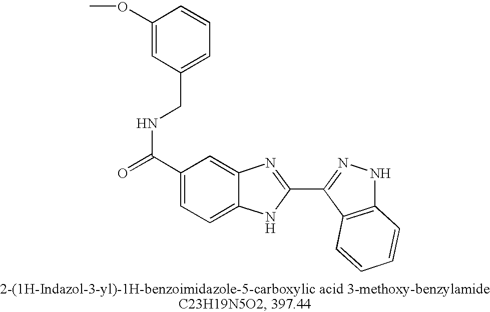 | 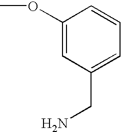 | 398 | 3.1 |
| 41 | 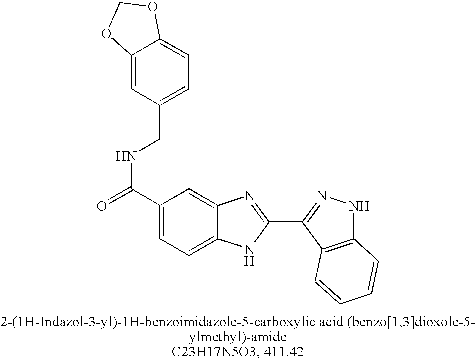 | 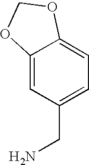 | 412 | 3.07 |
| 42 | 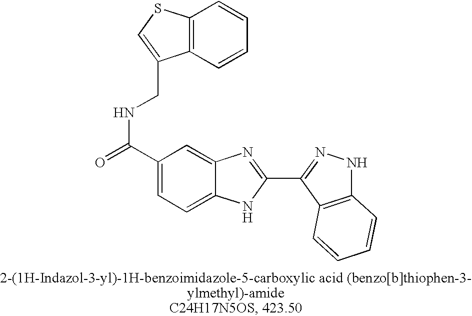 | 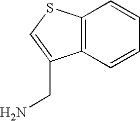 | 424 | 3.42 |
| 43 | 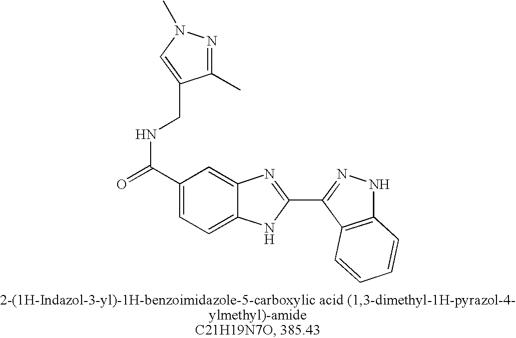 | 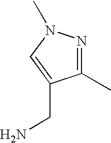 | 386 | 2.59 |
| 44 | 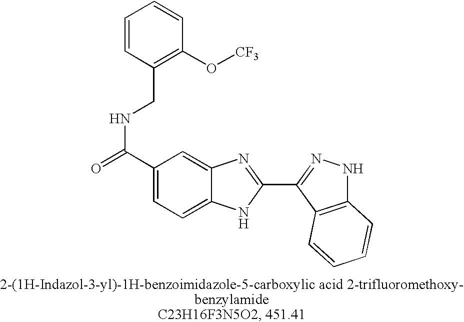 | 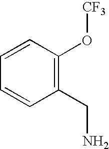 | 452 | 3.44 |
| 45 | 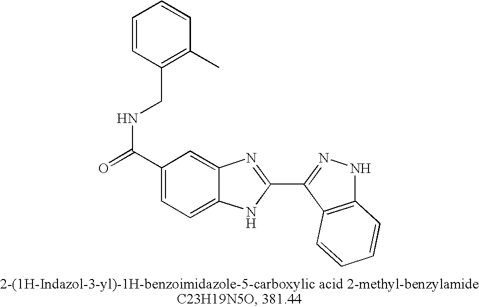 | 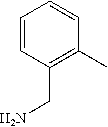 | 382 | 3.21 |
| 46 | 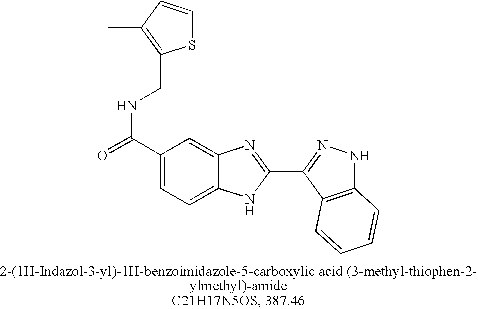 | 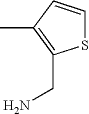 | 388 | 3.16 |
| 47 | 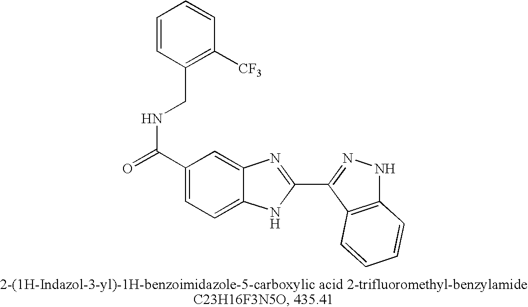 | 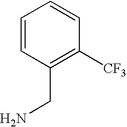 | 436 | 3.38 |
| 48 | 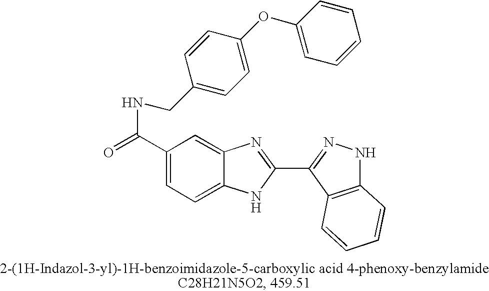 | 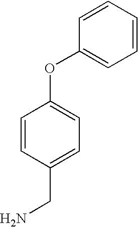 | 460 | 3.56 |
| 49 | 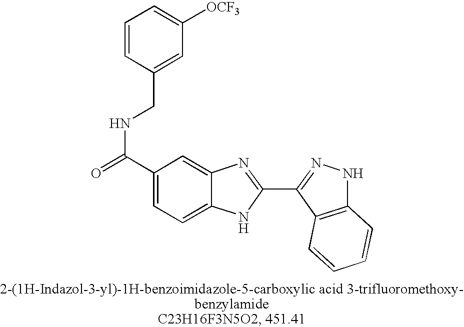 | 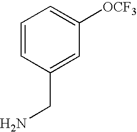 | 452 | 3.46 |
| 50 | 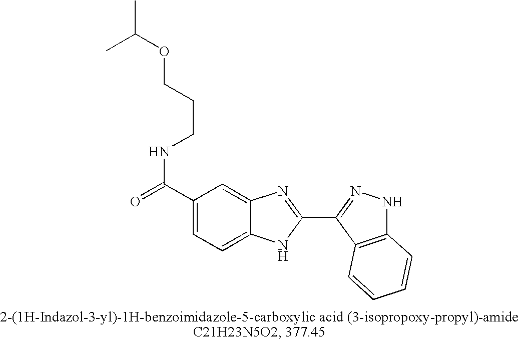 | 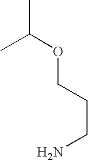 | 378 | 2.94 |
| 51 | 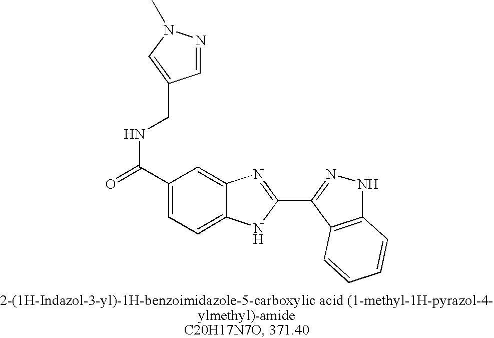 | 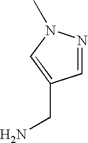 | 372 | 2.56 |
| 52 | 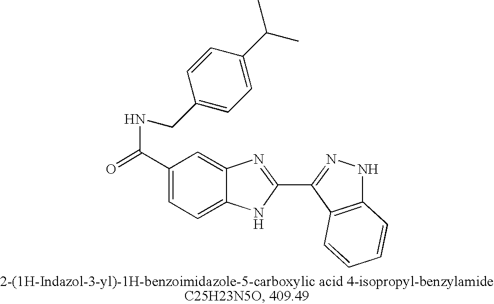 | 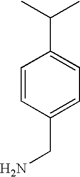 | 410 | 3.51 |
| 53 | 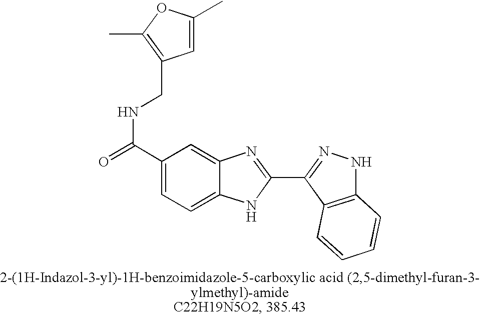 | 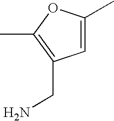 | 386 | 3.19 |
| 54 | 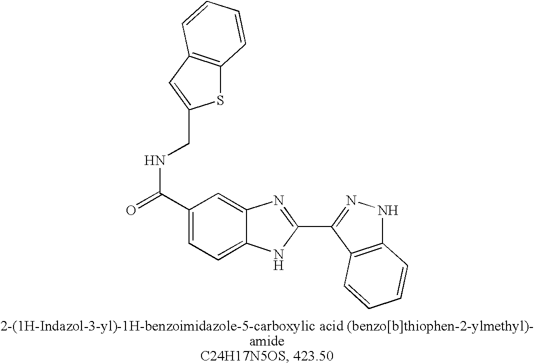 | 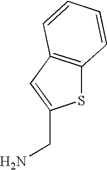 | 424 | 3.38 |
| 55 | 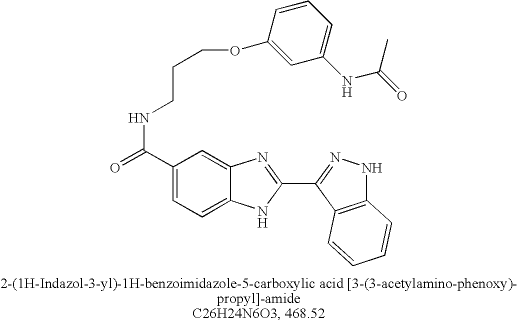 | 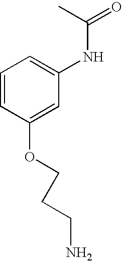 | 469 | 2.92 |
| 56 | 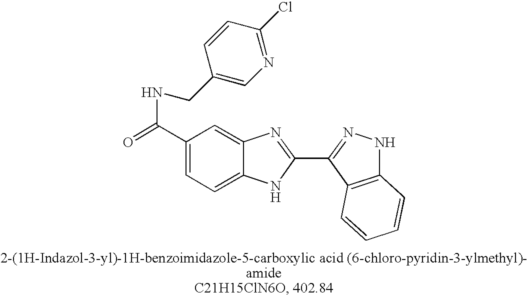 | 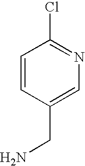 | 403 | 2.92 |
| 57 | 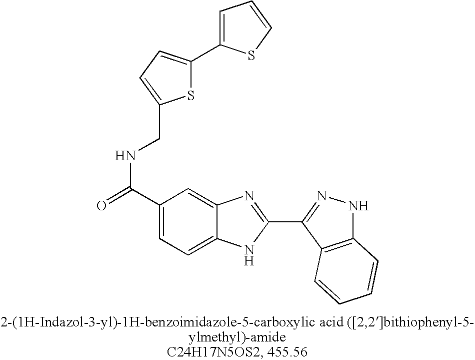 | 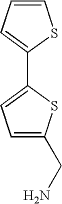 | 456 | 3.47 |
| 58 | 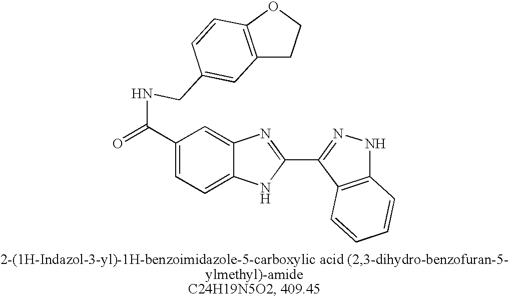 | 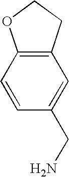 | 410 | 3.07 |
| 59 | 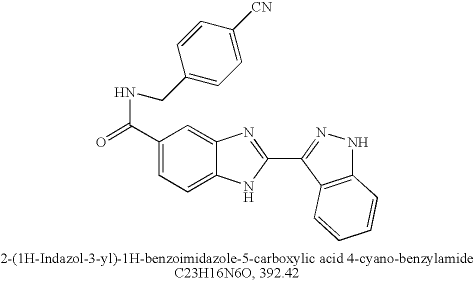 | 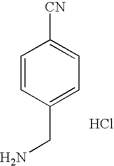 | 393 | 3.03 |
| 60 | 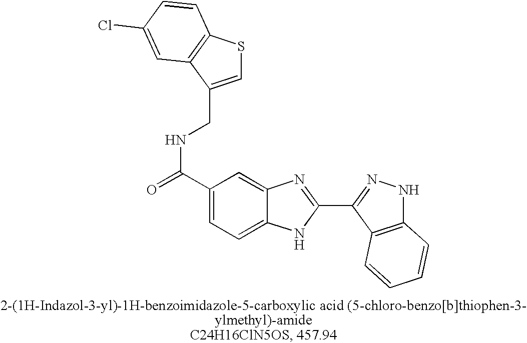 | 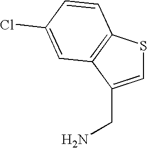 | 458 | 3.55 |
| 61 | 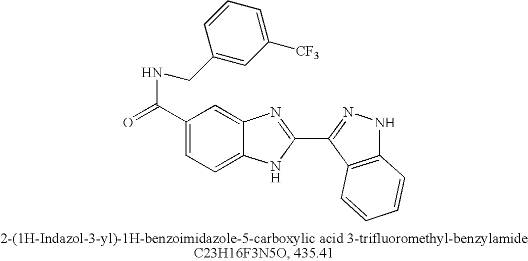 | 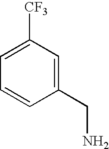 | 436 | 3.41 |
| 62 | 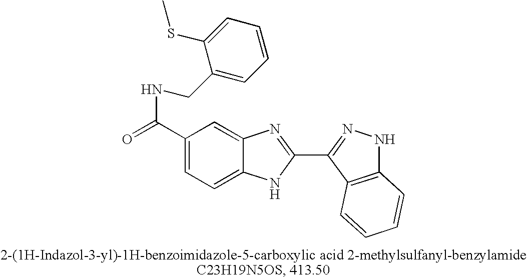 | 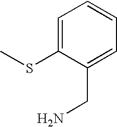 | 414 | 3.26 |
| 63 | 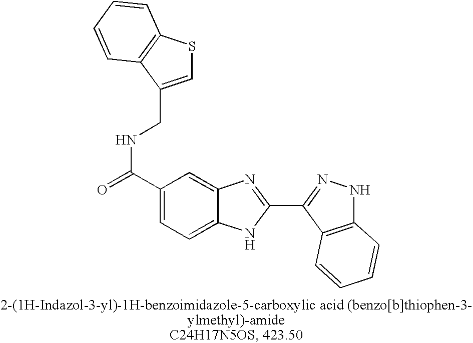 | 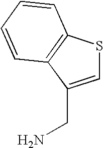 | 424 | 3.38 |
| 64 | 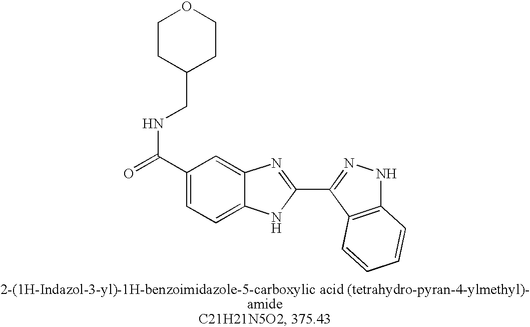 | 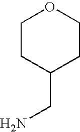 | 376 | 2.65 |
| 65 | 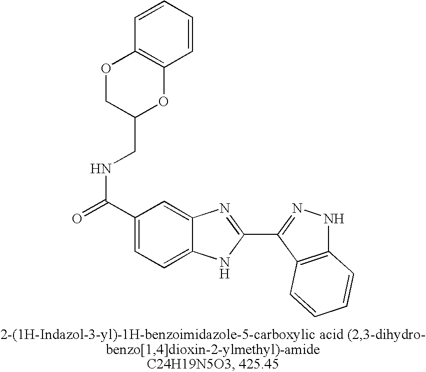 | 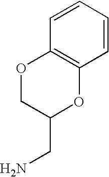 | 426 | 3.28 |
| 66 | 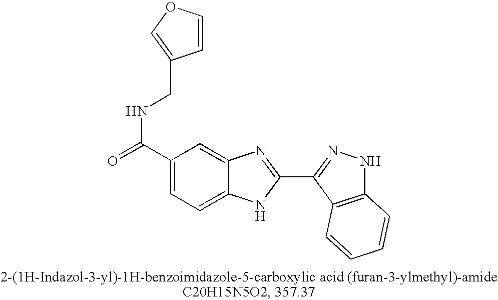 | 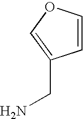 | 358 | 2.92 |
| 67 | 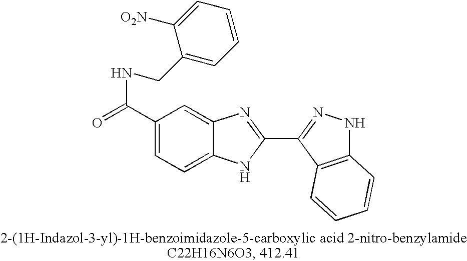 | 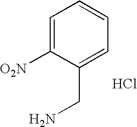 | 413 | 3.14 |
| 68 | 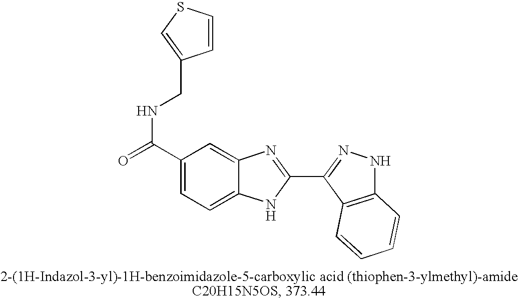 | 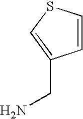 | 374 | 3.03 |
| 69 | 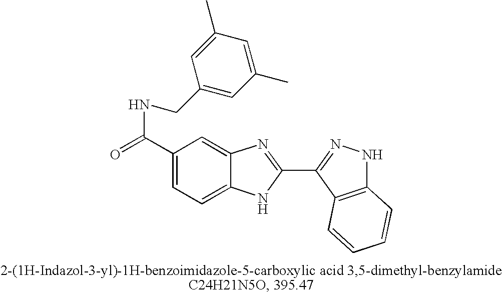 | 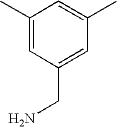 | 396 | 3.37 |
| 70 | 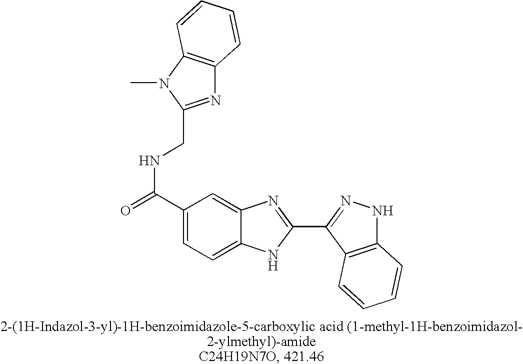 | 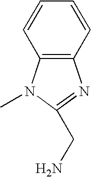 | 422 | 2.61 |
| 71 | 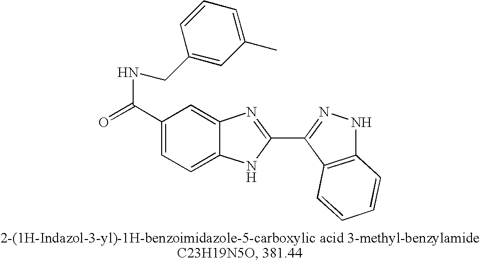 | 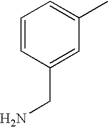 | 382 | 3.24 |
| 72 | 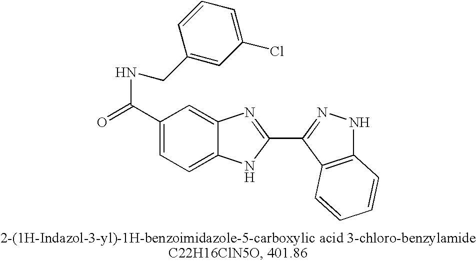 | 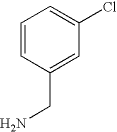 | 402 | 3.29 |
| 73 | 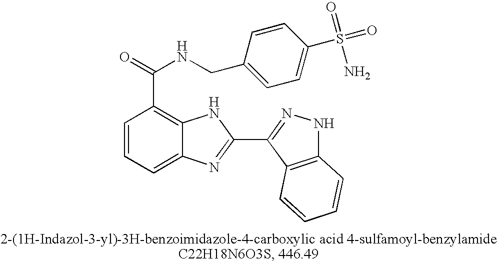 | 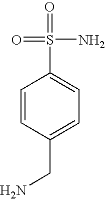 | 447 | 3.07 |
| 74 | 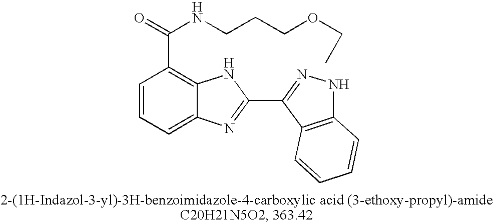 | 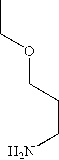 | 364 | 3.45 |
| 75 | 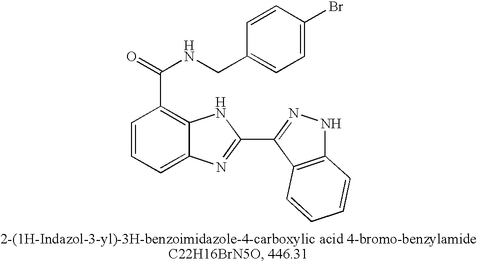 | 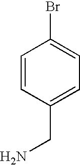 | 447 | 4.38 |
| 76 | 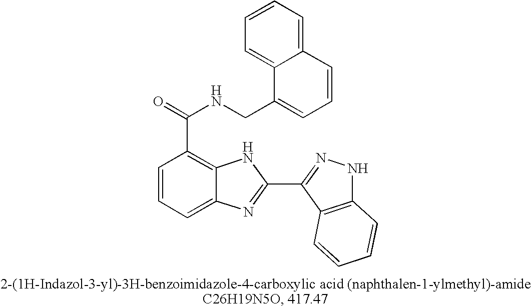 | 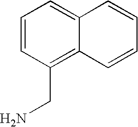 | 418 | 4.4 |
| 77 | 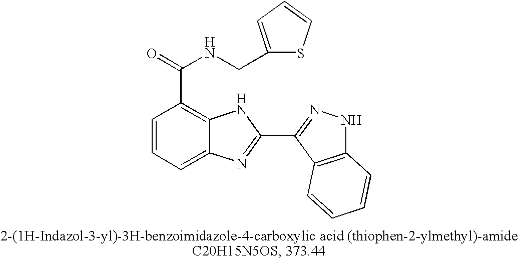 | 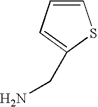 | 374 | 3.93 |
| 78 | 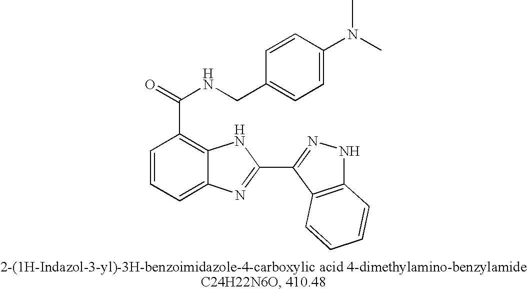 | 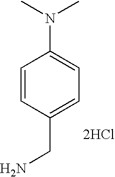 | 411 | 2.93 |
| 79 | 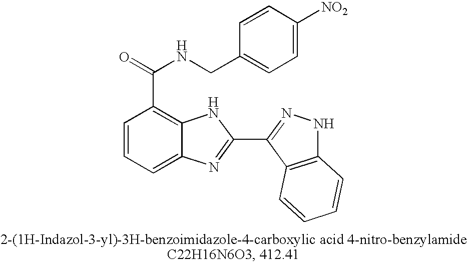 | 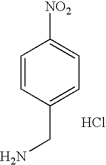 | 413 | 3.87 |
| 80 | 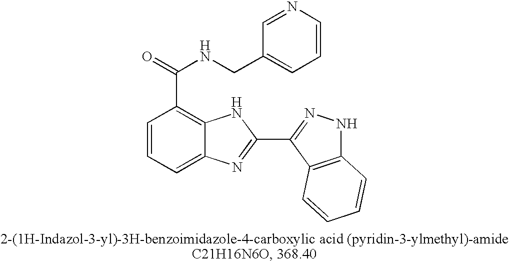 | 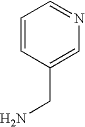 | 369 | 2.4 |
| 81 | 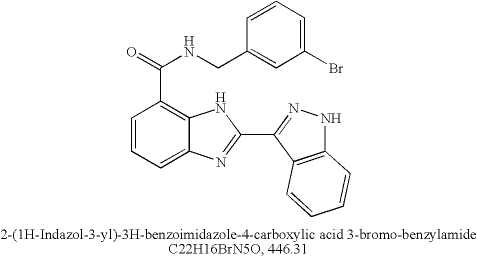 | 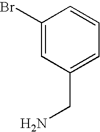 | 447 | 4.18 |
| 82 | 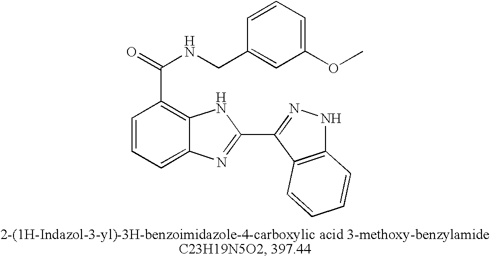 | 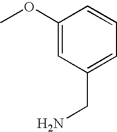 | 398 | 3.95 |
| 83 | 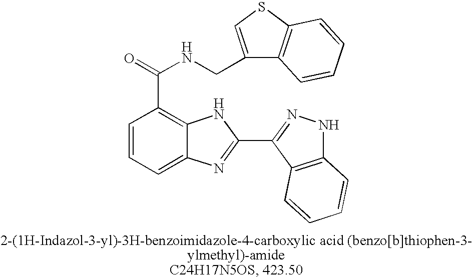 | 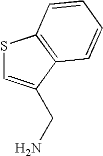 | 424 | 4.68 |
| 84 | 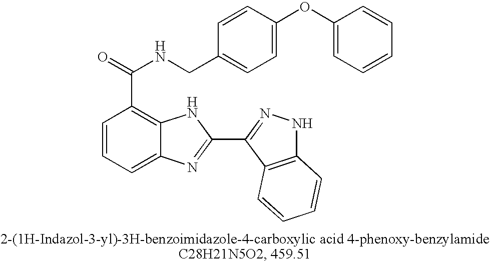 | 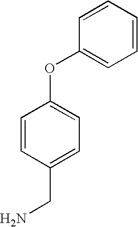 | 460 | 4.55 |
| 85 | 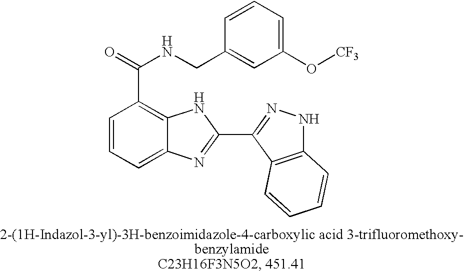 | 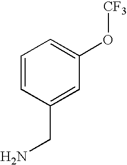 | 452 | 4.43 |
| 86 | 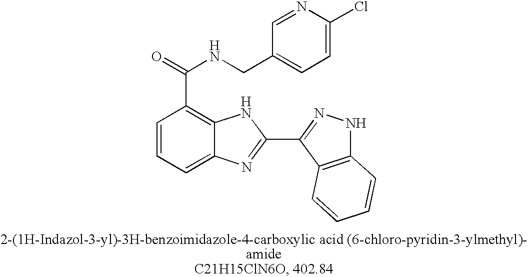 | 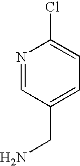 | 403 | 3.9 |
| 87 | 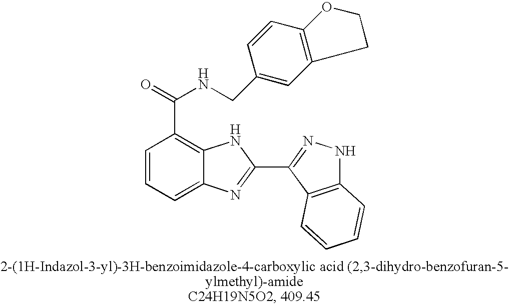 | 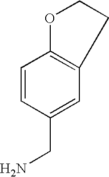 | 410 | 3.9 |
| 88 | 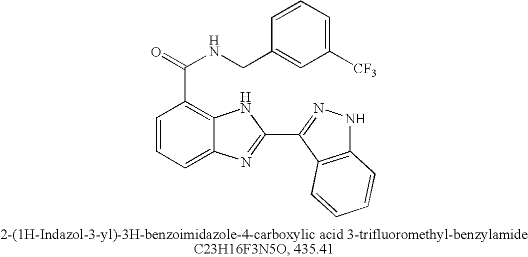 | 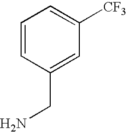 | 436 | 4.3 |
| 89 | 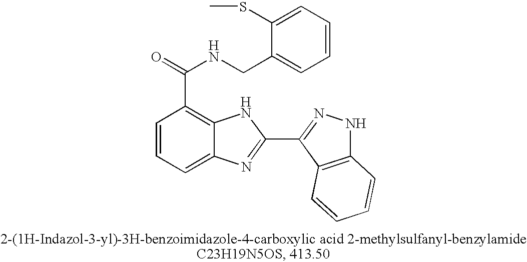 | 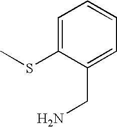 | 414 | 3.98 |
| 90 | 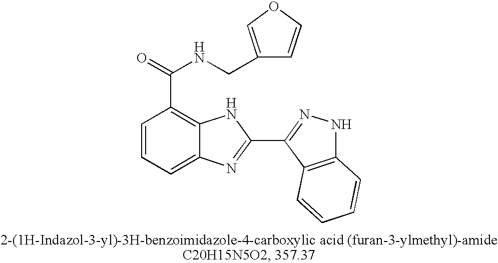 | 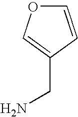 | 358 | 3.68 |
| 91 | 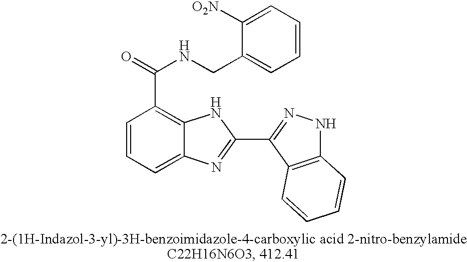 | 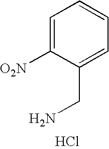 | 413 | 3.95 |
| 92 | 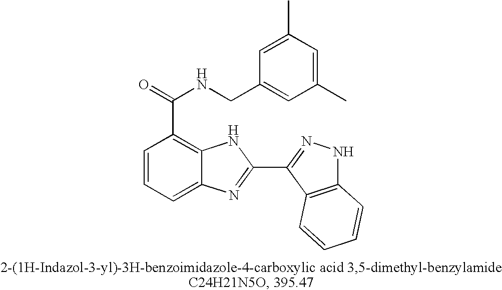 | 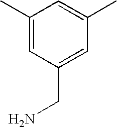 | 396 | 4.45 |
| 93 | 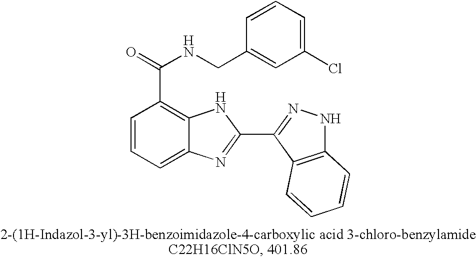 | 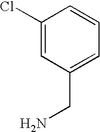 | 402 | 5.03 |
| 94 | 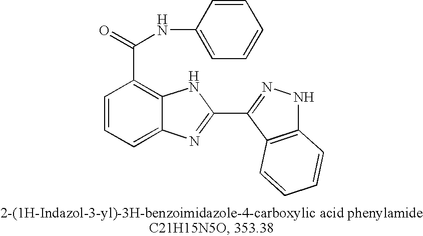 | 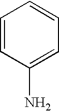 | 354 | 4.27 |
| 95 | 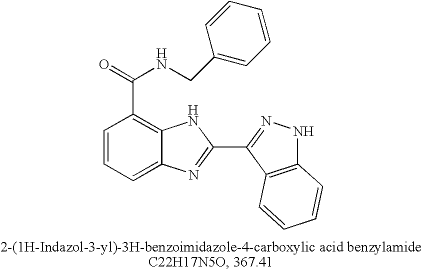 | 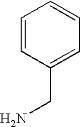 | 368 | 3.94 |
| 96 | 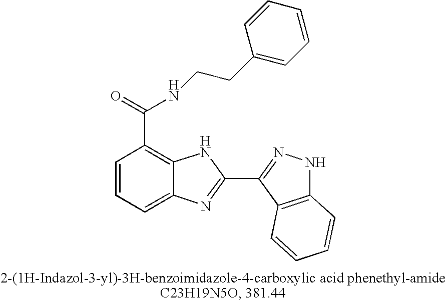 | 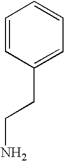 | 382 | 4.01 |
| 97 | 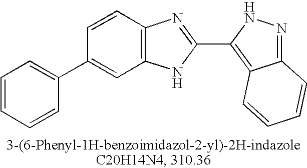 | 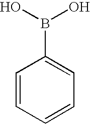 | 311 | 3.14 |
| 98 | 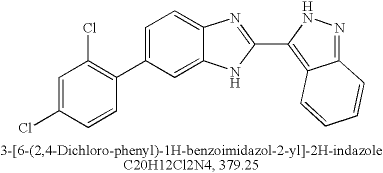 | 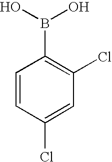 | 379 | 3.63 |
| 99 | 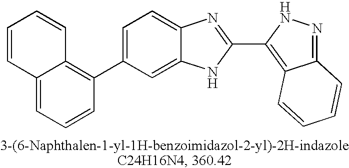 | 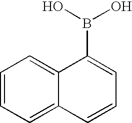 | 361 | 3.51 |
| 100 | 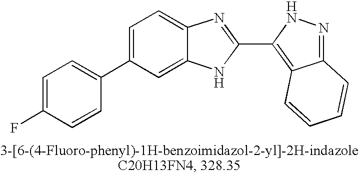 | 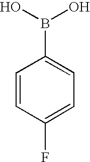 | 329 | 3.21 |
| 101 | 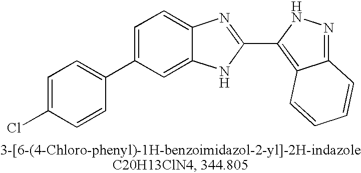 | 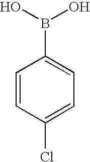 | 345 | 3.44 |
| 102 | 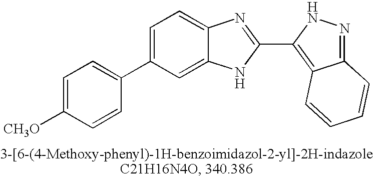 | 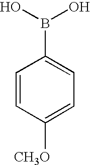 | 341 | 3.14 |
| 103 | 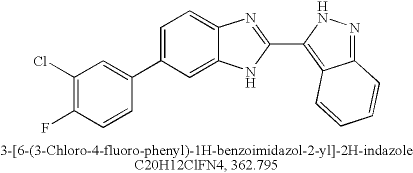 | 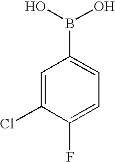 | 362 | 3.51 |
| 104 | 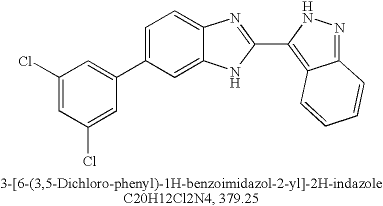 | 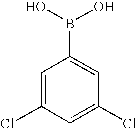 | [378-380] | 3.81 |
| 105 | 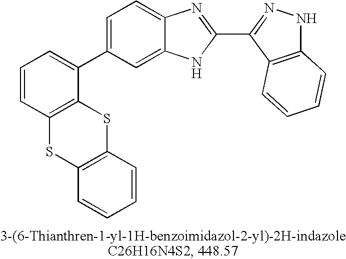 | 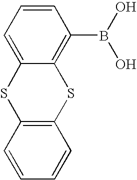 | 449 | 3.91 |
| 106 | 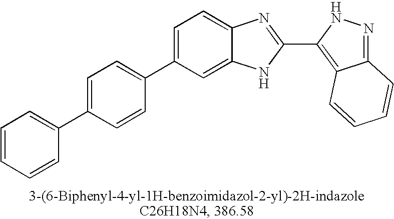 | 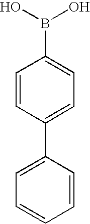 | 387 | 3.78 |
| 107 | 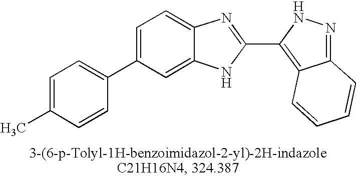 | 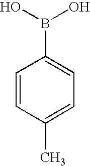 | 324[M]⁺ | 3.38 |
| 108 | 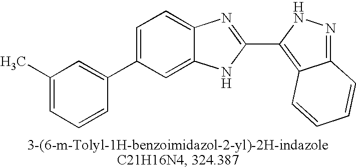 | 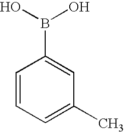 | 325 | 3.41 |
| 109 | 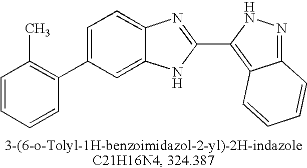 | 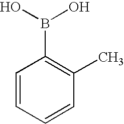 | 325 | 3.41 |
| 110 | 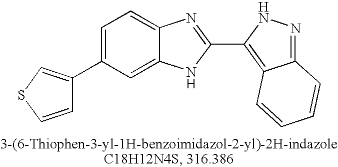 | 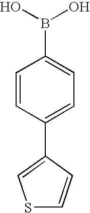 | 317 | 3.13 |
| 111 | 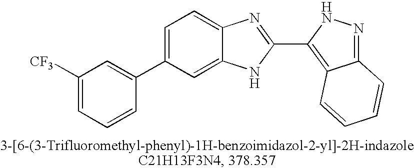 | 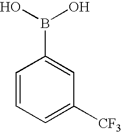 | 379 | 3.65 |
| 112 | 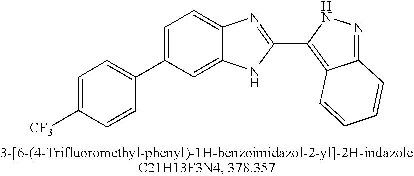 | 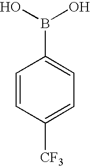 | 379 | 3.68 |
| 113 | 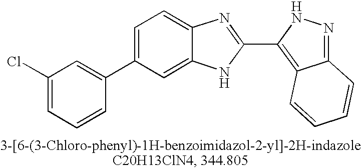 | 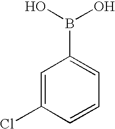 | 345 | 3.55 |
| 114 | 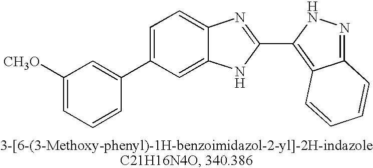 | 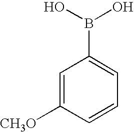 | 341 | 3.41 |
| 115 | 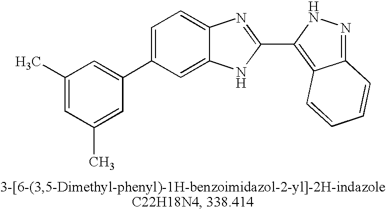 | 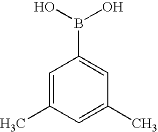 | 339 | 3.39 |
| 116 | 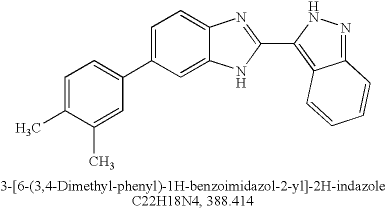 | 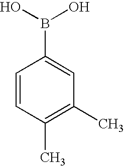 | 339 | 3.55 |
| 117 | 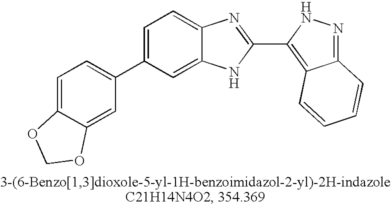 | 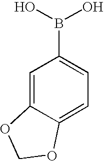 | 354[M]⁺ | 3.18 |
| 118 | 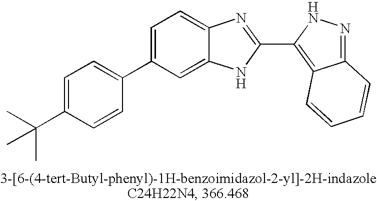 | 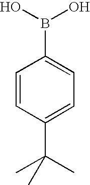 | 367 | 3.95 |
| 119 | 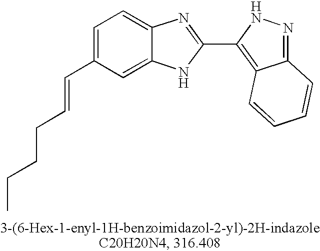 | 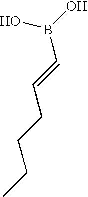 | 317 | 3.72 |
| 120 | 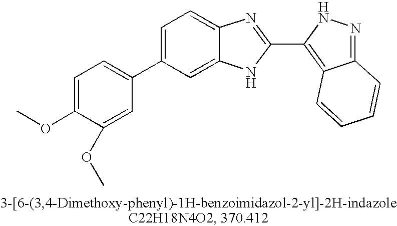 | 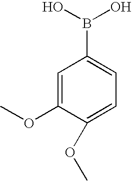 | 371 | 3.00 |
| 121 | 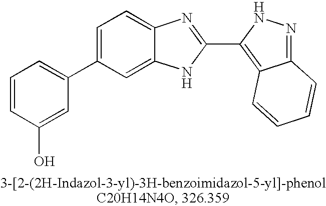 | 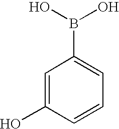 | 327 | 2.92 |
| 122 | 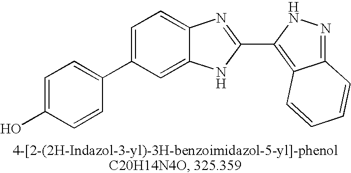 | 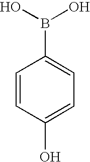 | 327 | 2.84 |
| 123 | 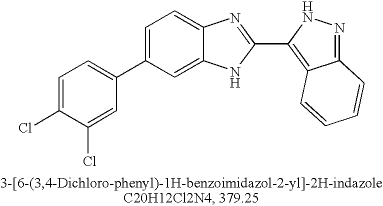 | 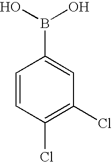 | 378 | 3.82 |
| 124 | 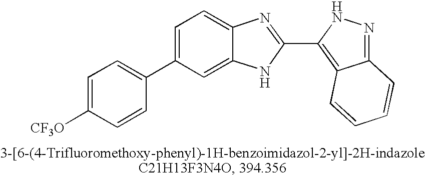 | 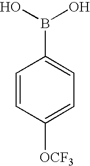 | 395 | 3.72 |
| 125 | 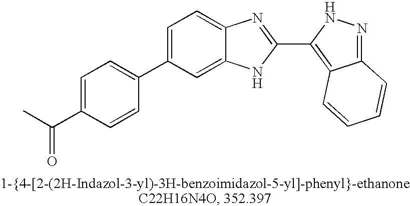 | 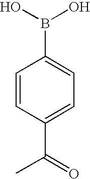 | 353 | 3.08 |
| 126 | 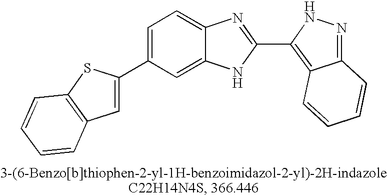 | 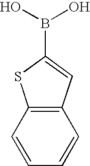 | 367 | 3.82 |
| 127 | 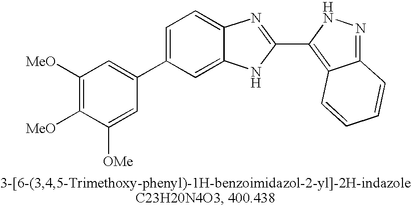 | 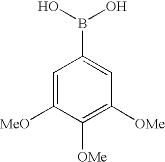 | 401 | 3.02 |
| 128 | 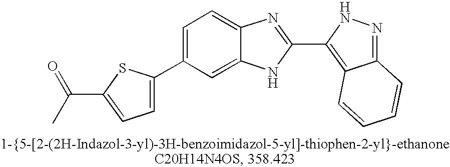 | 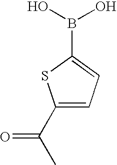 | 359 | 3.09 |
| 129 | 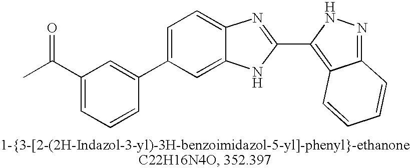 | 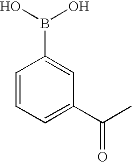 | 353 | 3.05 |
| 130 | 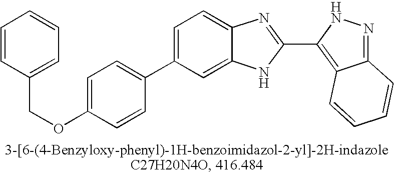 | 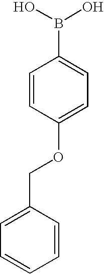 | 417 | 3.75 |
| 131 | 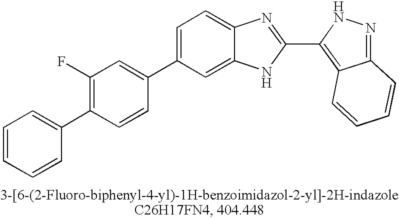 | 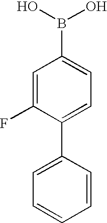 | 405 | 4.02 |
| 132 | 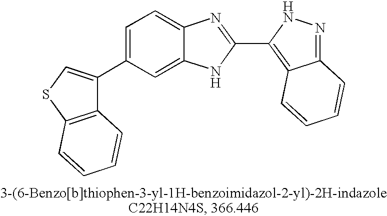 | 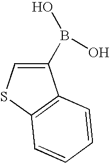 | 367 | 3.55 |
| 133 | 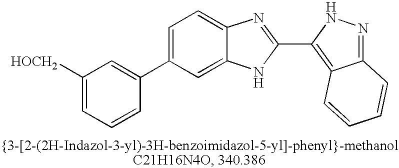 | 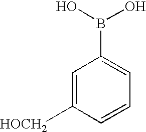 | 341 | 2.79 |
| 134 | 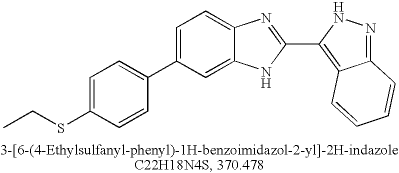 | 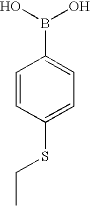 | 371 | 3.62 |
| 135 | 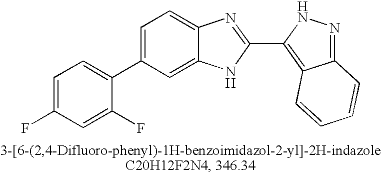 | 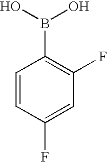 | 347 | 3.29 |
| 136 | 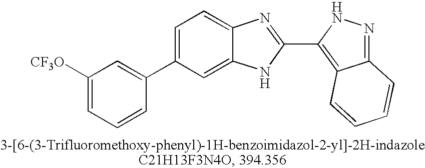 | 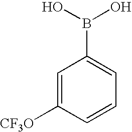 | 395 | 3.66 |
| 137 | 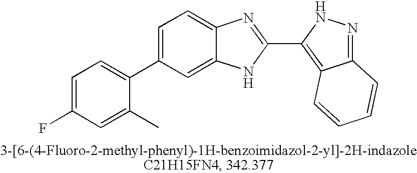 | 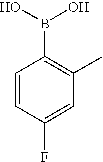 | 343 | 3.36 |
| 138 | 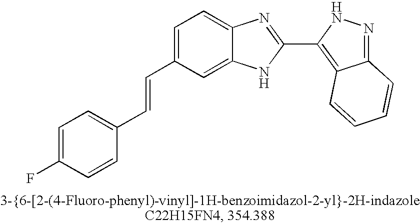 | 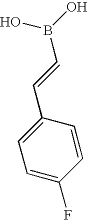 | 355 | 3.49 |
| 139 | 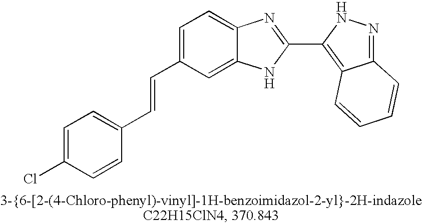 | 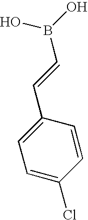 | 371 | 3.76 |
| 140 | 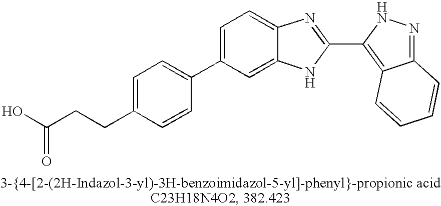 | 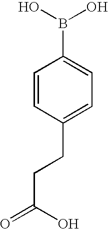 | 383 | 3.03 |
| 141 | 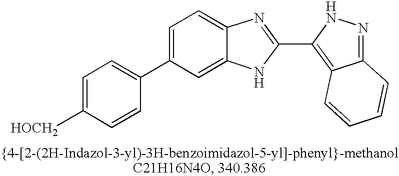 | 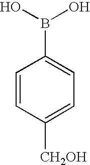 | 341 | 2.72 |
| 142 | 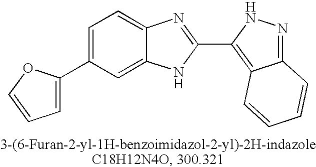 | 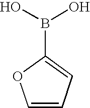 | 301 | 3.02 |
| 143 | 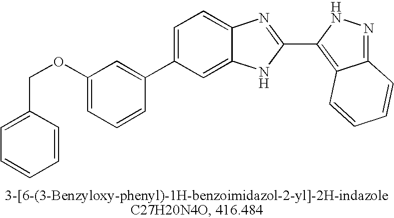 | 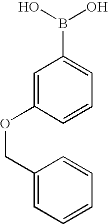 | 417 | 3.93 |
| 144 | 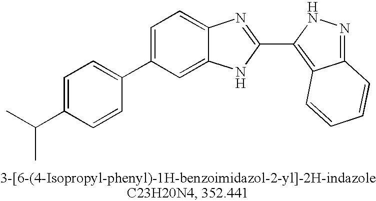 | 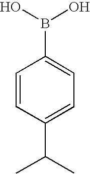 | 353 | 3.88 |
| 145 | 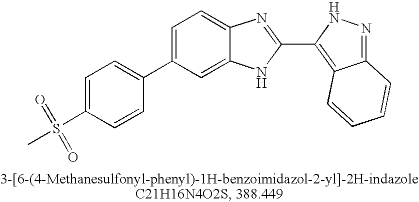 | 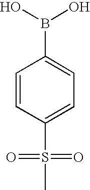 | 389 | 3.03 |
| 146 | 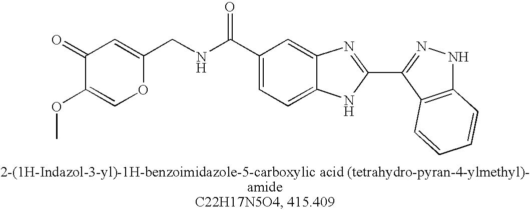 | 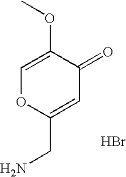 | 415[M]⁺ | 2.31 |
| 147 | 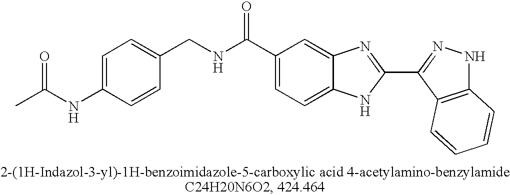 | 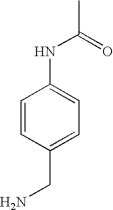 | 424[M]⁺ | 2.58 |
| 148 | 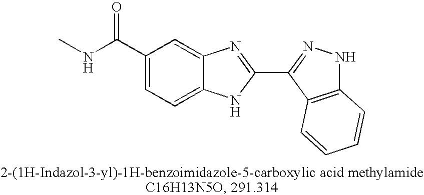 | CH₃NH₂ | 291[M]⁺ | 2.22 |
| 149 | 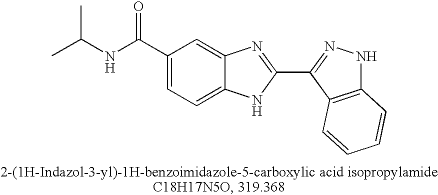 | (CH₃)₂CHNH₂ | 319[M]⁺ | 2.63 |
| 150 | 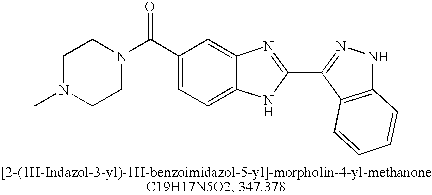 | 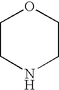 | 347[M]⁺ | 2.23 |
| 151 | 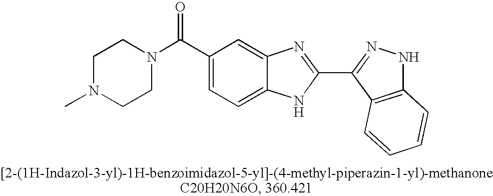 | 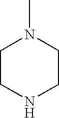 | 361 | 1.94 |
| 152 | 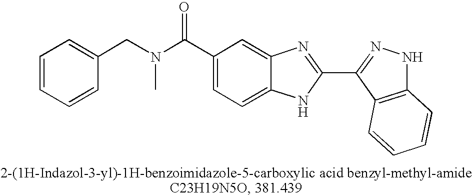 | 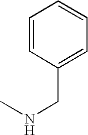 | 381[M]⁺ | 3.45 |
| 153 | 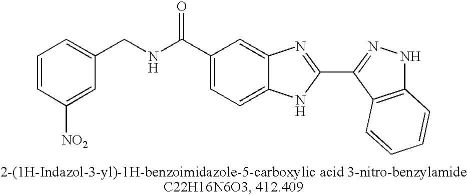 | 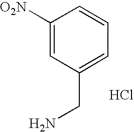 | 412[M]⁺ | 3.32 |
| 154 | 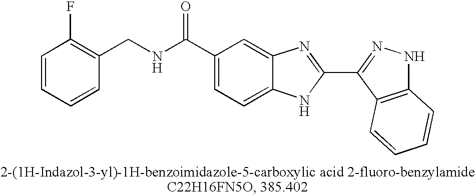 | 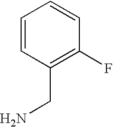 | 385[M]⁺ | 2.96 |
| 155 | 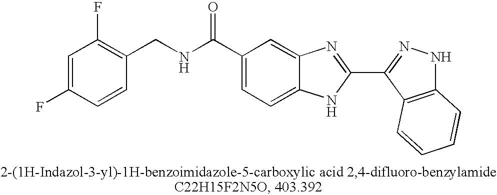 | 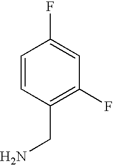 | 403[M]⁺ | 3.26 |
| 156 | 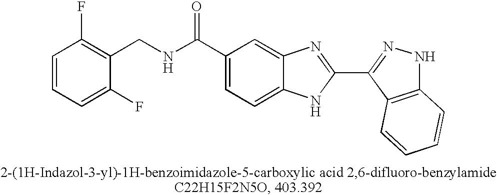 | 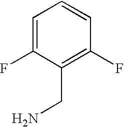 | 403[M]⁺ | 2.93 |
| 157 | 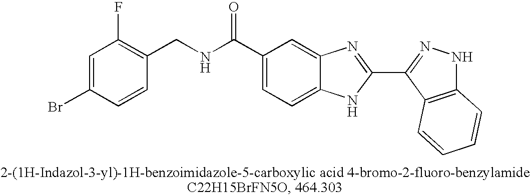 | 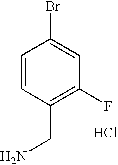 | 340[M]⁺ | 3.34 |
| 158 | 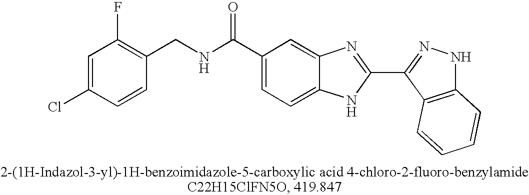 | 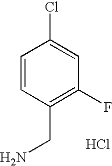 | 419[M]⁺ | 3.21 |
| 159 | 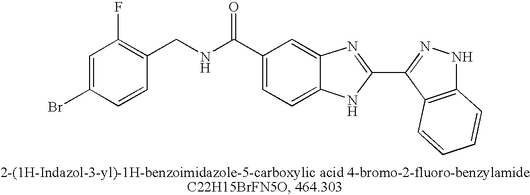 | 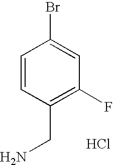 | 464[M]⁺ | 3.31 |
| 160 | 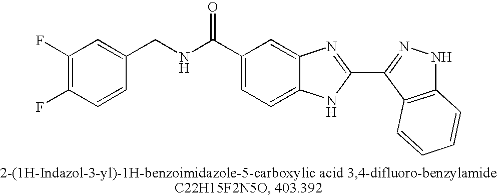 | 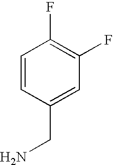 | 403[M]⁺ | 3.64 |
| 161 | 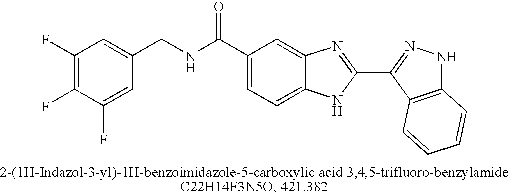 | 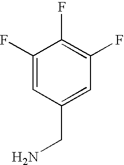 | 421[M]⁺ | 3.35 |
| 162 | 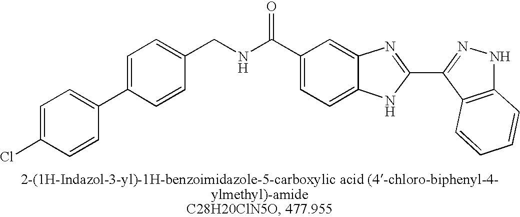 | 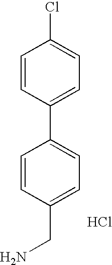 | 477[M]⁺ | 3.89 |
| 163 | 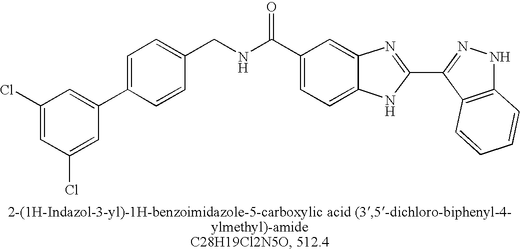 | 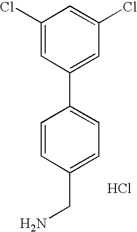 | 512[M]⁺ | 4.36 |
| 164 | 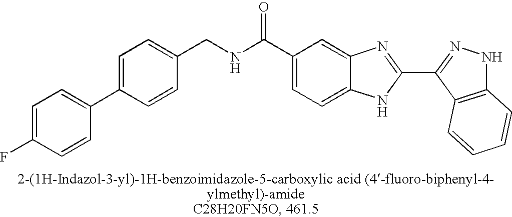 | 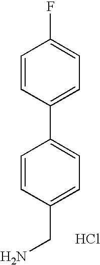 | 461[M]⁺ | 3.6 |
| 165 | 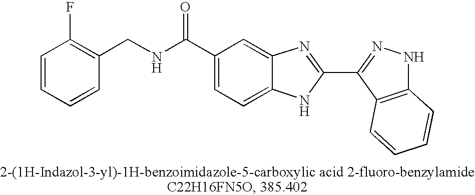 | 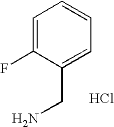 | 385[M]⁺ | 2.94 |
| 166 | 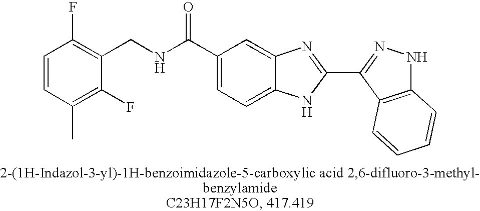 | 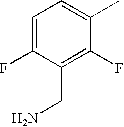 | 417[M]⁺ | 3.14 |
| 167 | 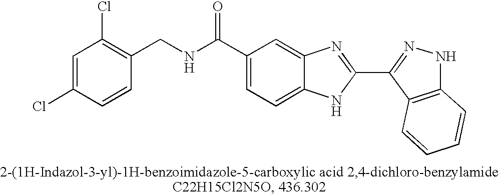 | 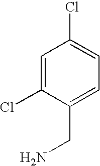 | 436[M]⁺ | 3.48 |
| 168 | 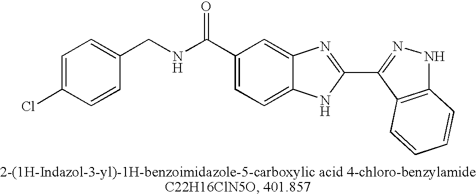 | 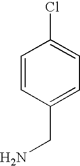 | 401[M]⁺ | 3.73 |
| 169 | 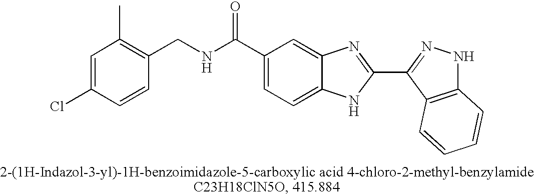 | 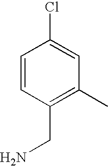 | 415[M]⁺ | 3.52 |
| 170 | 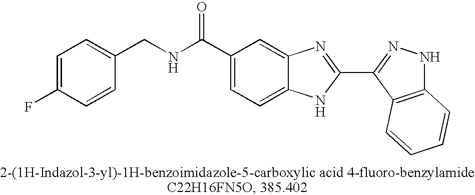 | 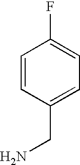 | 385[M]⁺ | 3.09 |
| 171 | 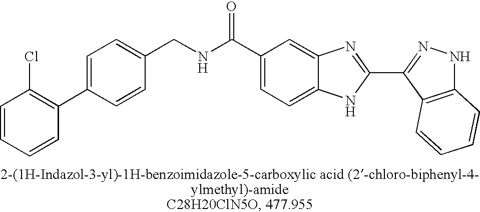 | 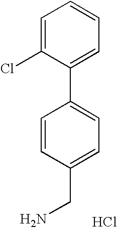 | 477[M]⁺ | 3.9 |
| 172 | 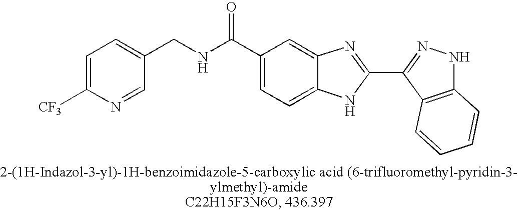 | 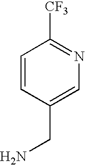 | 436[M]⁺ | 2.93 |
| 173 | 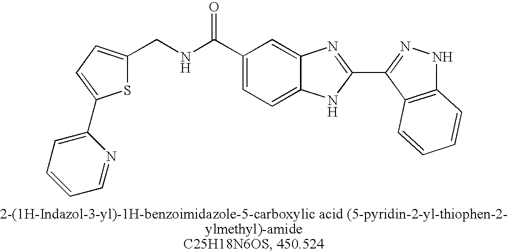 | 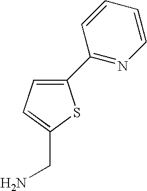 | 450[M]⁺ | 2.67 |
| 174 | 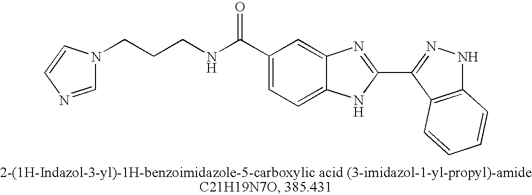 | 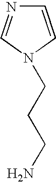 | 385[M]⁺ | 2.11 |
| 175 | 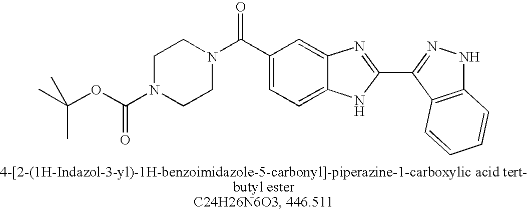 | 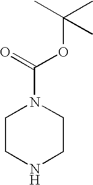 | 447 | 3.11 |
| 176 | 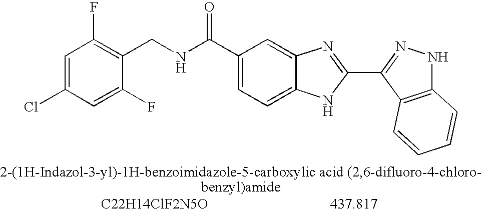 | 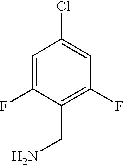 | 438 | |
| 177 | 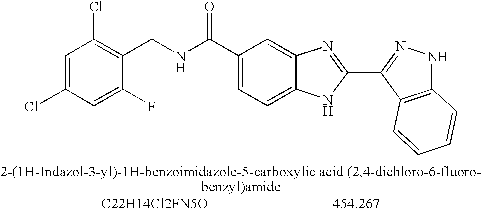 | 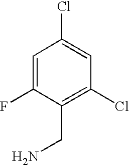 | 437 | |
| 178 | 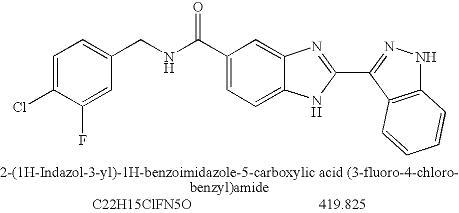 | 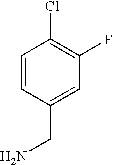 | 420 | |
| 179 | 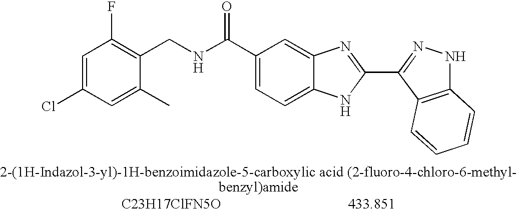 | 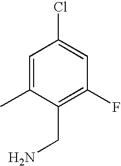 | 434 | |
| 180 | 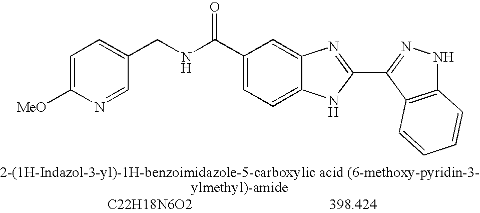 | 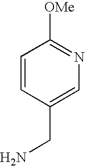 | 399 | |

The products of formula (I) of the present invention can also be prepared according to the following process:

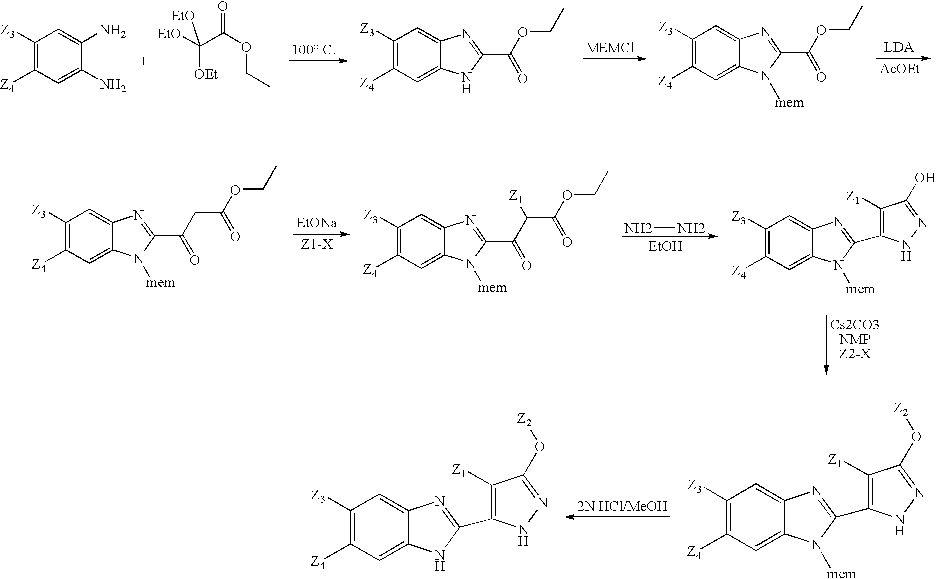

In the above scheme, the values of Z3 and Z4 are chosen from the values of R2 and R3 as defined above and the values of Z1 and —OZ2 are chosen from the values of X1, X2 or X3 with R1 represents a pyrazole radical,

When Z1, Z3 and Z4 represent a hydrogen atom, it is possible in particular to prepare products of formula (I) of the present invention according to the following synthesis scheme:

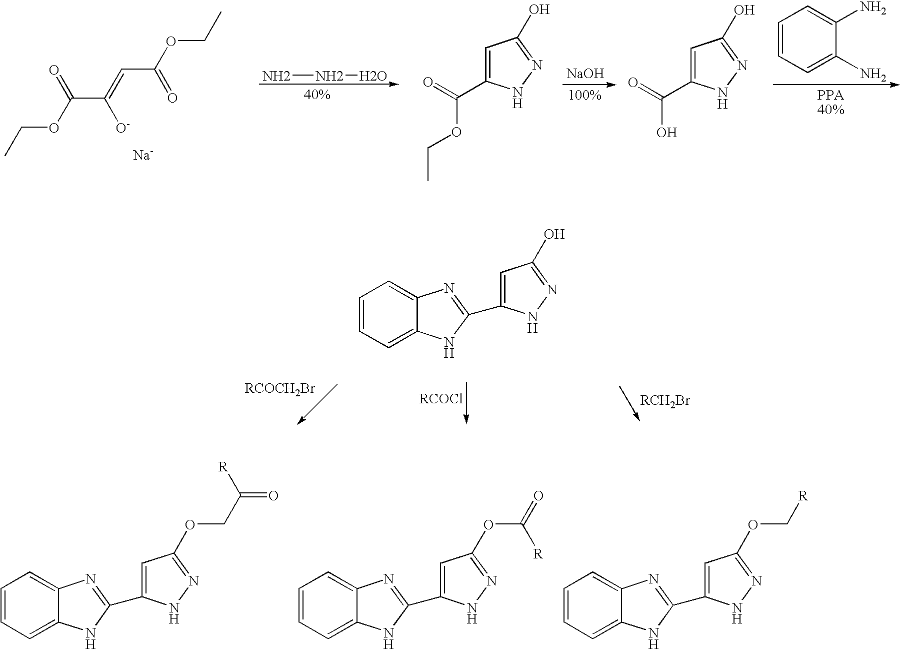

The products of formula (I) of the present invention constituted by Examples 181 to 228 of the present invention are represented in the table V. These products can be prepared as indicated in the schemes above and in particular the product of Example 181 can be prepared as indicated below. The products of Examples 182 to 228 can be prepared like the product of Example 181.

### Example 181

### 2-[5-(benzyloxy)-2H-pyrazol-3-yl]-1H-benzoimidazole

Step 1: the cyclization is performed as described in the papers: Chem. Pharm. Bull., 31(4), 1228-1234 (1983); J. Org. Chem., 47(2), 214-221 (1982).

Step 2: to the crude ester of 1.015 g obtained in step 1 in 50 ml of MeOH is added 5.5 ml of 6N NaOH and the mixture is heated to reflux during 2 hours. After evaporation of the methanol, the medium is cooled and conc. HCl is added until pH=2 is obtained. After evaporation to dryness, the solid is taken up three times with 30 ml of MeOH/AcOEt 1/1 and the filtrate is evaporated to give 0.875 g of a light brown solid after dessication.

LC/MS: [gradient acetonitrile/water 0.1% HCOOH; Xterra RP18 2.1×50 mm] retention time 0.53 min MH+=129 95% pure

Step 3: to 3.5 g of PPA (polyphosphoric acid) are added 0.701 g of 1,2-phenylenediamine and 0.87 g of the acid obtained above in step 2. The mixture is heated to 150° C. during 1.5 hours. After cooling, conc NH4OH is added until pH=3. The green precipitate is filtered, washed with water and then with acetone. After one night drying under vacuum at 50° C., 2.1 g of solid are obtained containing around 50% of mineral salts.

MS: EI M⁺=200

Step 4: to 80 mg of the product obtained in step 3 above in 4 ml of NMP are added 137 mg of caesium carbonate and 72 mg of benzyl bromide. After 2 hours, the mixture is hydrolysed with saturated KH2PO4 and extracted with AcOEt. After evaporation, the mixture is submitted to preparative LC/MS to give 8 mg of pure product.

LC/MS: [gradient acetonitrile/water 0.1% HCOOH; Xterra RP18 2.1×50 mm] retention time 3.17 min MH⁺=291 97% pure

In the same way as in Example 181, step 4 is carried out with 15 benzyl or allyl bromides, 15 α-bromocarbonyl products and 15 acid chlorides in DMF or in NMP. The expected corresponding products are obtained of which Examples 181 to 228 of the present invention are represented in table V below.

TABLE V

| Structure/Name of Starting Material | | |
|---|---|---|
| CHEMISTRY | No. | Compound Name |
| 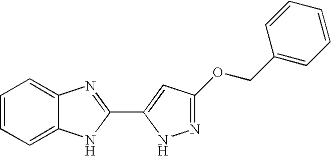 | 181 | 2-[5-(benzyloxy)-2H-pyrazol-3-yl]-1H-benzoimidazole |
| 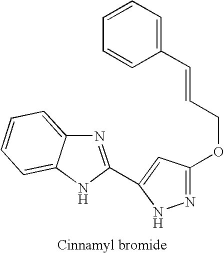 | 182 | 2-[5-(3-Phenyl-allyloxy)-2H-pyrazol-3-yl]-1H-benzoimidazole |
| 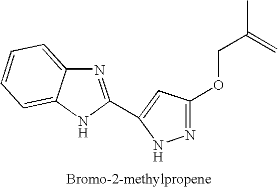 | 183 | 2-[5-(2-Methyl-allyloxy)-2H-pyrazol-3-yl]-1H-benzoimidazole |
| 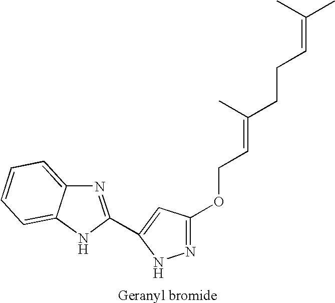 | 184 | 2-[5-(3,7-Dimethyl-octa-2,6-dienyloxy)-2H-pyrazol-3-yl]-1H-benzoimidazole |
| 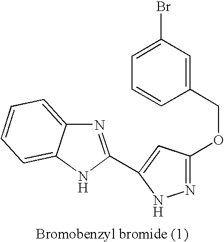 | 185 | 2-[5-(3-Bromo-benzyloxy)-2H-pyrazol-3-yl]-1H-benzoimidazole |
| 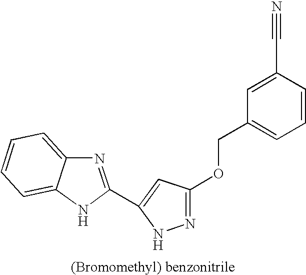 | 186 | 3-[5-(1H-Benzoimidazol-2-yl)-1H-pyrazol-3-yloxymethyl]-benzonitrile |
| 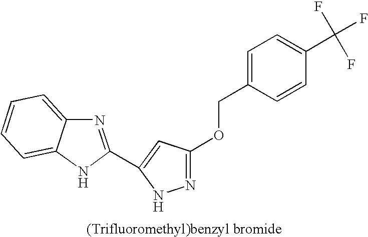 | 187 | 2-[5-(4-Trifluoromethyl-benzyloxy)-2H-pyrazol-3-yl]-1H-benzoimidazole |
| 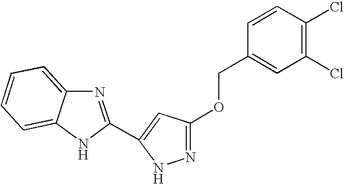 | 188 | 2-[5-(3,4-Dichloro-benzyloxy)-2H-pyrazol-3-yl]-1H-benzoimidazole |
| 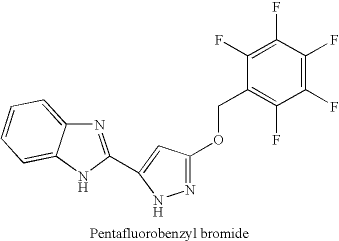 | 189 | 2-(5-Pentafluoro-phenylmethoxy-2H-pyrazol-3-yl)-1H-benzoimidazole |
| 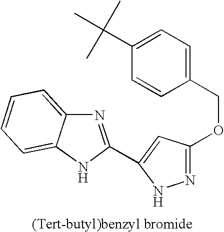 | 190 | 2-[5-(4-tert-Butyl-benzyloxy)-2H-pyrazol-3-yl]-1H-benzoimidazole |
| 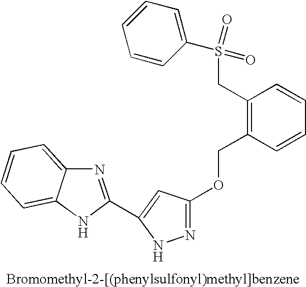 | 191 | 2-[5-(2-Benzenesulfonylmethylbenzyloxy)-2H-pyrazol-3-yl]-1H-benzoimidazole |
| 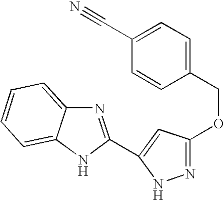 | 192 | 4-[5-(1H-Benzoimidazol-2-yl)-1H-pyrazol-3-yloxymethyl]-benzonitrile |
| 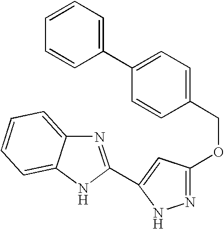 | 193 | 2-[5-(Biphenyl-4-ylmethoxy)-2H-pyrazol-3-yl]-1H-benzoimidazole |
| 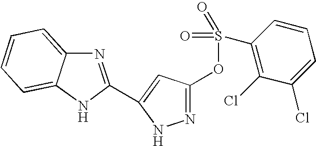 | 194 | 2,3-Dichloro-benzenesulfonic acid 5-(1H-benzoimidazol-2-yl)-1H-pyrazol-3-yl ester |
| 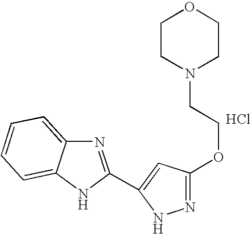 | 195 | 2-[5-(2-Morpholin-4-yl-ethoxy)-2H-pyrazol-3-yl]-1H-benzoimidazole |
| 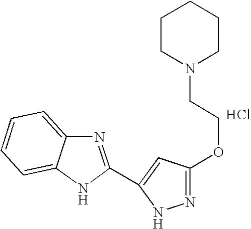 | 196 | 2-[5-(2-Piperidin-1-yl-ethoxy)-2H-pyrazol-3-yl]-1H-benzoimidazole |
| 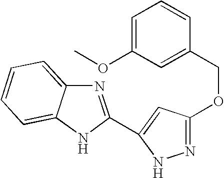 | 197 | 2-[5-(3-Methoxy-benzyloxy)-2H-pyrazol-3-yl]-1H-benzoimidazole |
| 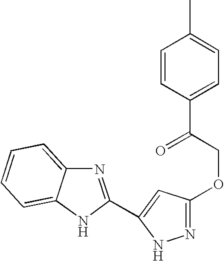 | 198 | 2-[5-(1H-Benzoimidazol-2-yl)-1H-pyrazol-3-yloxy]-1-p-tolyl-ethanone |
| 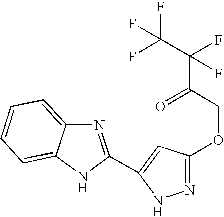 | 199 | 1-[5-(1H-Benzoimidazol-2-yl)-1H-pyrazol-3-yloxy]-3,3,4,4,4-pentafluoro-butan-2-one |
| 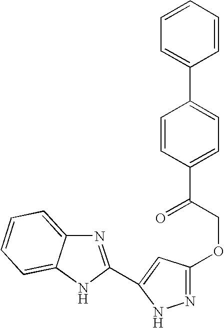 | 200 | 2-[5-(1H-Benzoimidazol-2-yl)-1H-pyrazol-3-yloxy]-1-biphenyl-4-yl-ethanone |
| 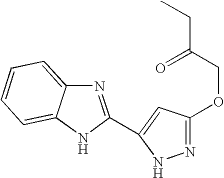 | 201 | 1-[5-(1H-Benzoimidazol-2-yl)-1H-pyrazol-3-yloxy]-butan-2-one |
| 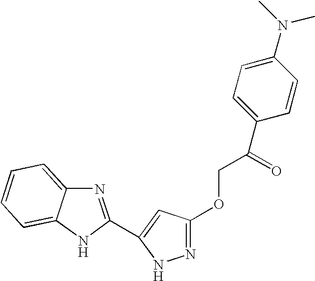 | 202 | 2-[5-(1H-Benzoimidazol-2-yl)-1H-pyrazol-3-yloxy]-1-(4-dimethylamino-phenyl)-ethanone |
| 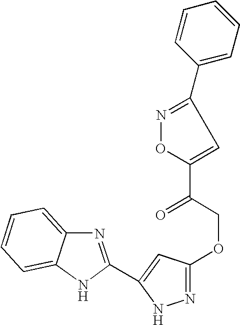 | 203 | 2-[5-(1H-Benzoimidazol-2-yl)-1H-pyrazol-3-yloxy]-1-(3-phenyl-isoxazol-5-yl)-ethanone |
| 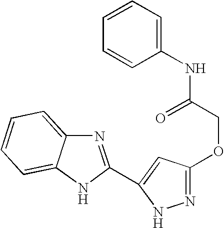 | 204 | 2-[5-(1H-Benzoimidazol-2-yl)-1H-pyrazol-3-yloxy]-N-phenyl-acetamide |
| 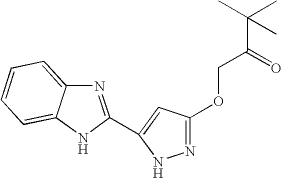 | 205 | 1-[5-(1H-Benzoimidazol-2-yl)-1H-pyrazol-3-yloxy]-3,3-dimethyl-butan-2-one |
| 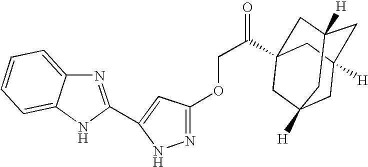 | 206 | 1-Adamantan-1-yl-2-[5-(1H-benzoimidazol-2-yl)-1H-pyrazol-3-yloxy]-ethanone |
| 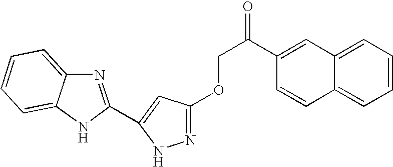 | 207 | 2-[5-(1H-Benzoimidazol-2-yl)-1H-pyrazol-3-yloxy]-1-naphthalen-2-yl-ethanone |
| 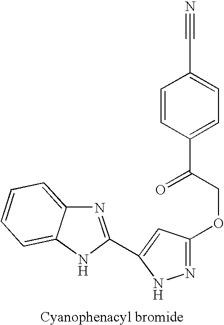 | 208 | 4-{2-[5-(1H-Benzoimi-dazol-2-yl)-1H-pyrazol-3-yloxy)acetyl}-benzonitrile |
| 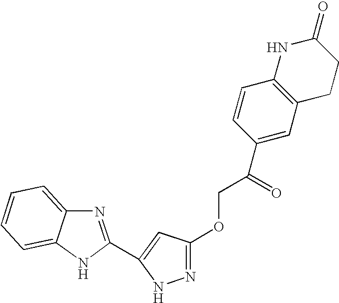 | 209 | 6-{2-[5-(1H-Benzoimi-dazol-2-yl)-1H-pyrazol-3-yloxy]-acetyl}-3,4-dihydro-1H-quinolin-2-one |
| 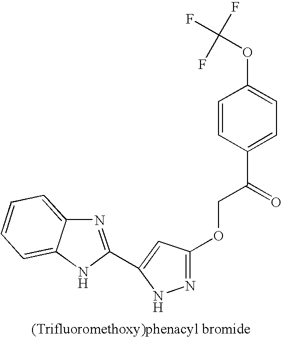 | 210 | 2-[5-(1H-Benzoimidazol-2-yl)-1H-pyrazol-3-yloxy]-1-(4-trifluoromethoxy-phenyl)-ethanone |
| 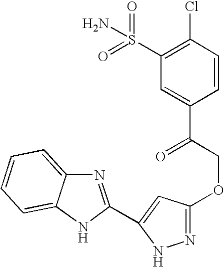 | 211 | 5-{2-[5-(1H-Benzoimida-zol-2-yl)-1H-pyrazol-3-yloxy]-acetyl}-2-chloro-benzenesulfonamide |
| 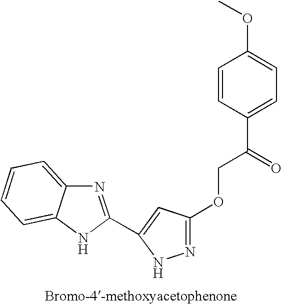 | 212 | 2-[5-(1H-Benzoimidazol-2-yl)-1H-pyrazol-3-yloxy]-1-(4-methoxy-phenyl)-ethanone |
| 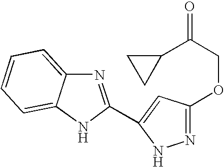 | 213 | 2-[5-(1H-Benzoimidazol-2-yl)-1H-pyrazol-3-yloxy]-1-cyclopropyl-ethanone |
| 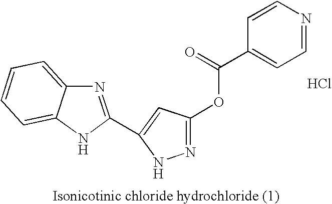 | 214 | Isonicotinic acid 5-(1H-benzoimidazol-2-yl)-1H-pyrazol-3-yl ester |
| 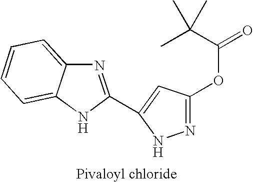 | 215 | 2,2-Dimethyl-propionicacid 5-(1H-benzoimidazol-2-yl)-1H-pyrazol-3-yl ester |
| 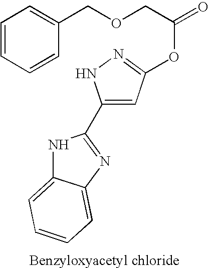 | 216 | Benzyloxy-acetic acid 5-(1H-benzoimidazol-2-yl)-1H-pyrazol-3-yl ester |
| 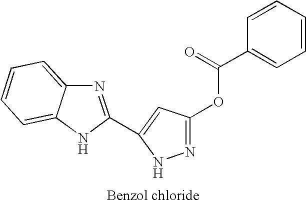 | 217 | Benzoic acid 5-(1H-benzoimidazol-2-yl)-1H-pyrazol-3-yl ester |
| 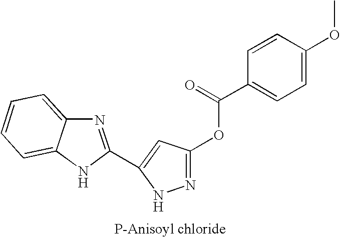 | 218 | 4-Methoxy-benzoic acid5-(1H-benzoimidazol-2-yl)-1H-pyrazol-3-yl ester |
| 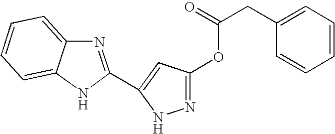 | 219 | Phenyl-acetic acid 5-(1H-benzoimidazol-2-yl)-1H-pyrazol-3-yl ester |
| 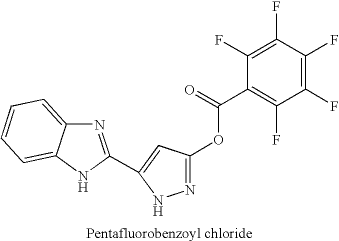 | 220 | 2,3,4,5,6-Pentafluoro-benzoic acid 5-(1H-benzoimidazol-2-yl)-1H-pyrazol-3-yl ester |
| 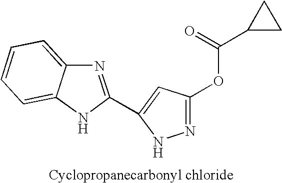 | 221 | Cyclopropanecarboxylicacid 5-(1H-benzoimidazol-2-yl)-1H-pyrazol-3-yl ester |
| 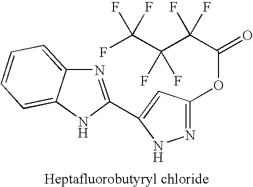 | 222 | 2,2,3,3,4,4,4-Heptafluoro-butyric acid5-(1H-benzoimidazol-2-yl)-1H-pyrazol-3-yl ester |
| 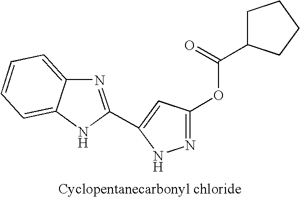 | 223 | Cyclopentanecarboxylicacid 5-(1H-benzoimidazol-2-yl)-1H-pyrazol-3-yl ester |
| 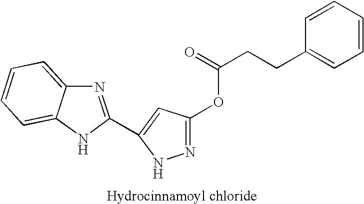 | 224 | 3-Phenyl-propionic acid5-(1H-benzoimidazol-2-yl)-1H-pyrazol-3-yl ester |
| 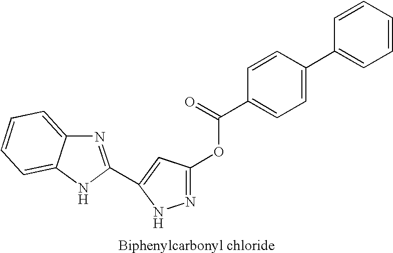 | 225 | Biphenyl-4-carboxylicacid 5-(1H-benzoimidazol-2-yl)-1H-pyrazol-3-yl ester |
| 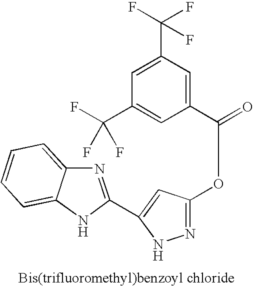 | 226 | 3,5-Bis-trifluoromethyl-benzoic acid 5-(1H-benzoimidazol-2-yl)-1H-pyrazol-3-yl ester |
| 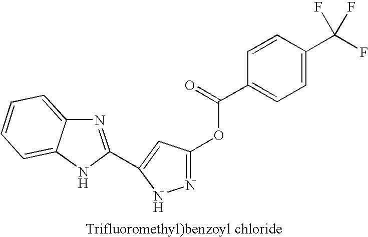 | 227 | 4-Trifluoromethyl-benzoic acid 5-(1H-benzoimidazol-2-yl)-1H-pyrazol-3-yl ester |
| 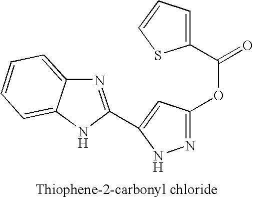 | 228 | Thiophene-2-carboxylicacid 5-(1H-benzoimidazol-2-yl)-1H-pyrazol-3-yl ester |

### Example 229

### Pharmaceutical Composition

Tablets corresponding to the formula below were prepared:

| | |
|---|---|
| Product of Example 1 | 0.2 g |
| Excipient for a finished tablet containing | 1 g |
| (details of the excipient: lactose, talc, starch, | |
| magnesium stearate). | |

Example 1 is taken as pharmaceutical preparation example, it being possible for this preparation to be produced, if desired, with other products in examples in the present invention.

### Biological Section

### In Vitro Test

Assessment of the inhibitory effect of the compounds on KDR:

### I) Biochemical Activity:

The inhibitory effect of the compounds is determined in a test of phosphorylation of a substrate by the enzyme KDR in vitro by the flasplate technique (96-well plate, NEN). The cytoplasmic domain of human KDR enzyme is cloned in the form of a GST fusion into the baculovirus expression vector pFastBac. The protein is expressed in the SF21 cells and purified to about 60% homogeneity.

The kinase activity of KDR is measured in 20 mM MOPS, 10 mM MgCl2, 10 mM MnCl2, 1 mM DTT, 2.5 mM EGTA, 10 mM β-glycerophosphate, pH 7.2 in the presence of 10 mM MgCl2, 100 μM Na3VO4, 1 mM NaF. 10 μl of the compound are added to 70 μl of kinase buffer containing 100 ng of KDR enzyme at 4° C. The reaction is initiated by adding 20 μl of solution containing 2 μg of substrate (fragment SH2-SH3 of PLCγ expressed in the form of a GST fusion protein), 2 μCi γ33P[ATP] and 2 μM cold ATP. After incubating for 1 h at 37° C., the reaction is quenched by adding 1 volume (100 μl) of 200 mM EDTA. The incubation buffer is removed and the wells are washed three times with 300 μl of PBS. The radioactivity is measured in each well using a Top Count NXT instrument (Packard).

Background noise is determined by measuring the radio-activity in wells in quadruplet containing radioactive ATP and the substrate alone.

An activity control is measured in wells in quadruplet containing all the reagents (γ33P-[ATP], KDR and the substrate PLCγ) and in the absence of compound.

The inhibition of the KDR activity with the compound of the invention is expressed as a percentage of inhibition of the control activity determined in the absence of compound.

The compound SU5614 (Calbiochem) (1 μM) is included in each plate as inhibition control.

The IC50 values for the compounds are calculated by plotting the dose-response curves. The IC₅₀ corresponds to the concentration of compound that induces a 50% inhibition of the kinase activity.

### II) Cellular Activity on Endothelial Cells

### 1) Inhibition of the VEGF-Dependent Proliferation of HDMECs

The anti-KDR activity of the molecules is assessed by incorporating [14C]-thymidine into HDMECs (Human Dermal Microvascular Endothelial Cells) in response to VEGF. HDMECs (Promocell, passage 5 to 7) are inoculated in 100 μl at 5000 cells per well in Cytostar (Amersham) 96-well plates precoated with attachment factor (AF, Cascad Biologics) at 37° C., 5% CO2, on day 1. On day 2, the complete medium (basal medium supplemented with 5% FCS and a mixture of growth factors) is replaced with minimum medium (basal medium supplemented with 5% FCS) and the cells are incubated for 24 hours. On day 3, the medium is replaced with 200 μl of fresh medium that has or has not been supplemented with 100 ng/ml of VEGF (R&D System) and containing or not containing the compound of the invention and 0.1 μCi [14C]-thymidine. The cells are incubated at 37° C. under 5% CO2 for 4 days. The incorporation of [14C]-thymidine is then quantified by counting the radioactivity. The tests are performed in 3 wells. The final concentration of DMSO in the test is 0.1%. The % of inhibition is calculated as follows: [cpm(+VEGF)−cpm (+VEGF+cpd)/cpm(+VEGF)−cpm (BM5% FCS)]×100.

### 2) Inhibition of the Production of TF (Tissue Factor) by Endothelial Cells in Response to VEGF

The endothelial cells are inoculated at 20,000 cells per well in a 96-well plate precoated with attachment factor. After culturing for 8 hours, the medium is changed and the cells are preincubated with the compounds (0.1% DMSO final) in basal medium for 16 hours. The synthesis of the TF (tissue factor) is induced by adding VEGF (100 ng/ml final). After incubating for 6 hours, the cells are rinsed and lysed. The tissue factor is then detected by means of the Imubind ELISA test.

### 3) Effect of the Molecules on the VEGF-Independent Growth of HDMECs

The HDMECs (5000 cells per well) are inoculated in complete medium in Cytostar (Amersham) 96-well plates precoated with attachment factor (AF, Cascad Biologics) at 37° C., 5% CO2, on day 1. The whole medium is then removed and the cells are incubated in 200 μl of complete medium containing the molecules of the invention and [14C]-thymidine (0.1 μCi). The incorporation of the [14C]-thymidine is measured using a Wallac counter after incubating for 3 days. The % of inhibition is calculated as follows: [cpm(CM)−cpm (CM+cpd)/cpm(CM)]×100.

Table VI below gives the results obtained in the above tests for the products indicated as examples in the present patent invention.

TABLE VI

| | | % of inhibition of the |
|---|---|---|
| | IC50 (μM) on | phosphorylation of |
| | inhibition of the | PLCγ by KDR (product |
| | phosphorylation | tested at a |
| Example No. | of PLCγ by KDR | concentration of 10 μM) |
| 1 | 0.47 | |
| 2 | 0.45 | |
| 3 | — | 91.8 |
| 4 | 0.45 | |
| 5 | — | 91.9 |
| 6 | 0.33 | |
| 7 | 0.72 | |
| 8 | 0.67 | |
| 9 | 0.35 | |
| 10 | 0.34 | |
| 11 | 0.26 | |
| 12 | 0.16 | |
| 13 | 0.61 | |
| 14 | 1.2 | |
| 15 | 0.8 | |
| 16 | 2 | |
| 18 | — | 91.2 |
| 20 | 3.4 | |
| 21 | — | 35 |
| 23 | 2 | |

The pharmacological results obtained in the above tests for products indicated in examples in the present invention are given in table VII below, the degrees of activities of the products being indicated by + signs according to the ranges of activity indicated as follows i.e.:

### + for IC50 >3 μM

++ for IC50 >0.3 μM and IC50 <3 μM micromolar

### +++ IC50 <0.3 μM

TABLE VII

| Ex. No. | Activity |
|---|---|
| 28 | +++ |
| 29 | ++ |
| 30 | +++ |
| 31 | +++ |
| 32 | ++ |
| 33 | ++ |
| 34 | ++ |
| 35 | ++ |
| 36 | ++ |
| 37 | +++ |
| 38 | ++ |
| 39 | ++ |
| 40 | ++ |
| 41 | ++ |
| 42 | ++ |
| 43 | ++ |
| 44 | ++ |
| 45 | +++ |
| 46 | ++ |
| 47 | ++ |
| 48 | ++ |
| 49 | ++ |
| 50 | ++ |
| 51 | ++ |
| 52 | ++ |
| 53 | ++ |
| 54 | ++ |
| 55 | ++ |
| 56 | +++ |
| 57 | ++ |
| 58 | +++ |
| 59 | ++ |
| 60 | + |
| 61 | + |
| 62 | +++ |
| 63 | +++ |
| 64 | ++ |
| 65 | ++ |
| 66 | ++ |
| 67 | ++ |
| 68 | ++ |
| 69 | ++ |
| 70 | ++ |
| 71 | +++ |
| 72 | +++ |
| 73 | ++ |
| 74 | + |
| 75 | + |
| 76 | + |
| 77 | + |
| 78 | + |
| 79 | + |
| 80 | ++ |
| 81 | + |
| 82 | ++ |
| 83 | + |
| 84 | + |
| 85 | + |
| 86 | + |
| 87 | + |
| 88 | + |
| 89 | +++ |
| 90 | ++ |
| 91 | ++ |
| 92 | ++ |
| 93 | + |
| 94 | ++ |
| 95 | + |
| 96 | + |
| 97 | + |
| 98 | + |
| 99 | + |
| 100 | ++ |
| 101 | + |
| 102 | ++ |
| 103 | ++ |
| 104 | + |
| 105 | + |
| 106 | + |
| 107 | + |
| 108 | ++ |
| 109 | ++ |
| 110 | ++ |
| 111 | + |
| 112 | + |
| 113 | ++ |
| 114 | ++ |
| 115 | ++ |
| 116 | + |
| 117 | ++ |
| 118 | + |
| 119 | ++ |
| 120 | ++ |
| 121 | ++ |
| 122 | ++ |
| 123 | + |
| 124 | + |
| 125 | + |
| 126 | + |
| 127 | ++ |
| 128 | ++ |
| 129 | + |
| 130 | + |
| 131 | + |
| 132 | + |
| 133 | ++ |
| 134 | + |
| 135 | ++ |
| 136 | + |
| 137 | ++ |
| 138 | + |
| 139 | + |
| 140 | + |
| 141 | ++ |
| 142 | ++ |
| 143 | + |
| 144 | ++ |
| 145 | + |
| 146 | ++ |
| 147 | ++ |
| 148 | ++ |
| 149 | ++ |
| 150 | ++ |
| 151 | ++ |
| 152 | ++ |
| 153 | ++ |
| 154 | ++ |
| 155 | ++ |
| 156 | ++ |
| 157 | +++ |
| 158 | ++ |
| 159 | ++ |
| 160 | ++ |
| 161 | ++ |
| 162 | + |
| 163 | + |
| 164 | + |
| 165 | + |
| 166 | ++ |
| 167 | +++ |
| 168 | +++ |
| 169 | +++ |
| 170 | ++ |
| 171 | ++ |
| 172 | ++ |
| 173 | ++ |
| 174 | ++ |
| 175 | ++ |
| 176 | +++ |
| 177 | +++ |
| 178 | +++ |
| 179 | +++ |
| 180 | +++ |
| 181 | ++ |
| 182 | ++ |
| 183 | + |
| 184 | ++ |
| 185 | ++ |
| 186 | + |
| 187 | + |
| 188 | ++ |
| 189 | + |
| 190 | + |
| 191 | ++ |
| 192 | + |
| 193 | ++ |
| 194 | + |
| 195 | + |
| 196 | + |
| 197 | ++ |
| 198 | + |
| 199 | + |
| 200 | + |
| 201 | + |
| 202 | + |
| 203 | + |
| 204 | + |
| 205 | + |
| 206 | + |
| 207 | + |
| 208 | + |
| 209 | + |
| 210 | + |
| 211 | + |
| 212 | + |
| 213 | + |
| 214 | ++ |
| 215 | + |
| 216 | + |
| 217 | ++ |
| 218 | + |
| 219 | + |
| 220 | + |
| 221 | + |
| 222 | + |
| 223 | + |
| 224 | ++ |
| 225 | + |
| 226 | + |
| 227 | + |
| 228 | + |

What is claimed is:

1) A compound of formula (I): 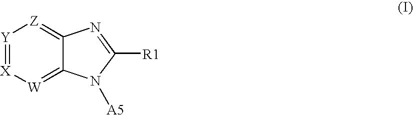
wherein
X is C—R2;
Y is C—R2 or C—R3
W and Z are each C—R3;
R1 is aryl or heteroaryl wherein heteroaryl is selected from the group consisting of pyrazol-4-yl, triazolyl, imidazolyl, indolyl, indazolyl, and thienopyrazolyl, wherein said aryl or heteroaryl is optionally substituted with one or more X1, X2 or X3 selected from the group consisting of H, halogen, haloalkyl, OH, R4, NO₂, CN, S(O)nR4, OR4, NY1Y2, COR4, —C(═O)NY1Y2, —C(═O)OR4, —C(═O)OH, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2, —OS(O)nR4, —OC(═O)R4 and optionally substituted thienyl; or
R1 is a saturated 5- or 6-membered monocyclic heterocyclic radical or a bicyclic heterocyclic radical having no more than 10 members wherein said monocyclic or bicyclic radicals contain at least two heteroatoms which are nitrogen and optionally contain other heteroatoms selected from the group consisting of O, N and S and wherein said monocyclic or bicyclic radicals are optionally substituted with one or more X1, X2 or X3 as defined above;
R2 and R3 may be identical or different and are selected independently of each other from the group consisting of H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2 and —OC(═O)R4, or
R2 is H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, N(R6)C(═O)OR4, —S(O)nOR4, —S(O)nNY1Y2, —OC(═O)NY1Y2 and —OC(═O)R4 and R3 is alkyl, haloalkyl, halogen or OR6, or
R2 and R3 together form a 5- to 6-membered ring containing one or more hetero atoms, which may be identical or different and selected from the group consisting of O, N and S;
R4 is alkyl, alkenyl, alkynyl, cycloalkyl, aryl, heteroaryl, cycloalkylalkyl, heterocycloalkyl, heteroarylalkyl or arylalkyl, wherein said alkyl, alkenyl, alkynyl, cycloalkyl, aryl, heteroaryl, cycloalkylalkyl, heterocycloalkyl, heteroarylalkyl or arylalkyl may be optionally substituted with one or more halogen, alkyl, hydroxyalkyl, OH, ORS, C(═O)NY3Y4, NY3Y4, alk-NY3Y4, C(═O)OR6 or optionally substituted aryl;
R5 and R6 may be identical or different and are each independently selected from the group consisting of alkyl, alkenyl, cycloalkyl, heterocycloalkyl, aryl, heteroaryl, arylalkyl, cycloalkylalkyl, heteroarylalkyl and heterocycloalkylalkyl;
Y1 and Y2 may be identical or different and are each independently selected from the group consisting of H and optionally substituted alkyl, alkenyl, cycloalkyl, heterocycloalkyl, heterocycloalkylalkyl, aryl, arylalkyl, heteroaryl, heteroarylalkyl, alkoxyalkyl and aryloxyalkyl; or
Y1 and Y2 together with the nitrogen atom to which they are attached form a ring;
Y3 and Y4 may be identical or different and are selected independently of each other from the group consisting of hydrogen, alkenyl, alkyl, aryl, arylalkyl, cycloalkyl, heteroaryl and heteroarylalkyl, or
Y3 and Y4 together with the nitrogen atom to which they are attached form an optionally substituted ring;
A5 is H or alkyl;
n is an integer from 0, 1 or 2;
wherein said alkyl, alk, alkenyl, cycloalkyl, heterocycloalkyl, aryl, arylalkyl, heteroaryl and heteroarylalkyl as defined above may be further optionally substituted with one or more halogen, hydroxyl, cyano, alkyl, alkoxy, acylamino (NH—COalk), —C(═O)OR6, acyl —C(═O)R6, hydroxyalkyl, carboxyalkyl, S(O)n-alk, S(O)n—NH2, S(O)n—NH(alk), S(O)n—N(alk)2, CF3, OCF3, NO2, arylalkoxy, aryl, heteroaryl, aryloxy, aryloxyalkyl, —C(═O)—NY3Y4 or NY3Y4, wherein the latter radicals containing alkyl, alkoxy, aryl or heteroaryl may be optionally substituted with one or more halogen, alkyl, acylamino or free, salified or esterified carboxyl; and
wherein said phenyl as defined above may be further optionally substituted by dioxole,
wherein when R1 is indazol-3-yl and the compound of formula (I) is the compound of formula (F) 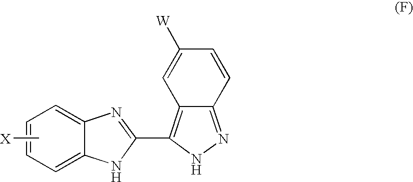
and X is H, R2 or R3 as defined above, then W is H or unsubstituted alkyl; or
a stereoisomer, a racemate, an enantiomer or a diastereoisomer of said compound, or
a pharmaceutically acceptable salt thereof.

2) The compound according to claim 1 wherein
R1 is pyrazol-4-yl, triazolyl or indazolyl;
R2 and R3 may be identical or different and are selected independently of each other from the group consisting of H, R4, halogen, OH, OR4, COR4, —C((═O)NY1Y2, —C(═O)OR4 and —C(═O)OH, or
R2 is H, R4, halogen, OH, OR4, —C(═O)NY1Y2, —C(═O)OR4, or —C(═O)OH, and R3 is alkyl, halogen or OR6, or
R2 and R3 together with the carbons to which they are attached form a methylenedioxybenzimidazole or a ethylenedioxybenzimidazole, and
R6 is H or C1-C4alkyl.

3) The compound of formula (I) according to claim 2 wherein R1 is indazolyl.

4) The compound of formula (I) according to claim 3 selected from the group consisting of:
2-(1H-indazol-3-yl)-1H-benzimidazole-5-carboxylic acid benzylamide,
2-(1H-indazol-3-yl)-1H-benzimidazole-5-carboxylic acid N-methylamide,
2-(1H-indazol-3-yl)-1H-benzimidazole-5-carboxylic acid N-ethylamide,
2-(1H-indazol-3-yl)-1H-benzimidazole-5-carboxylic acid N-isopropylamide,
2-(1H-indazol-3-yl)-1H-benzimidazole-5-carboxylic acid N-phenylamide,
2-(1H-indazol-3-yl)-1H-benzimidazole-5-carboxylic acid N-phenethylamide,
2-(1H-indazol-3-yl)-1H-benzimidazole-5-carboxylic acid N-morpholinoamide,
2-(1H-indazol-3-yl)-1H-benzimidazole-5-carboxylic acid N—(N′-methylpiperazino)amide,
2-(1H-indazol-3-yl)-1H-benzimidazole-5-carboxylic acid N-pyrrolidinoamide,
2-(1H-indazol-3-yl)-1H-benzimidazole-5-carboxylic acid N-(isobutyl)amide,
2-(1H-indazol-3-yl)-1H-benzimidazole-5-carboxylic acid N-(cyclohexylmethyl)amide,
2-(1H-indazol-3-yl)-1H-benzimidazole-5-carboxylic acid N-(2-furfuryl)amide,
2-(1H-indazol-3-yl)-1H-benzimidazole-5-carboxylic acid N-benzyl-N-methylamide,
methyl 2-(1H-indazol-3-yl)-3H-benzimidazole-5-carboxylate,
5,6-dimethyl-2-(1H-indazol-3-yl)-1H-benzimidazole,
5-methoxy-2-(1H-indazol-3-yl)-1H-benzimidazole,
2-(1H-indazol-3-yl)-3H-benzimidazole-4-carboxylic acid,
5-bromo 2-(1H-indazol-3-yl)-3H-benzimidazole,
2-(1H-indazol-3-yl)-1H-benzimidazole-5-carboxylic acid 4-(aminosulphonyl)benzylamide,
2-(1H-indazol-3-yl)-1H-benzimidazole-5-carboxylic acid 4-bromobenzylamide,
2-(1H-indazol-3-yl)-1H-benzimidazole-5-carboxylic acid 4-(methanesulphonyl)benzylamide,
2-(1H-indazol-3-yl)-1H-benzimidazole-5-carboxylic acid 4-nitrobenzylamide,
2-(1H-indazol-3-yl)-1H-benzimidazole-5-carboxylic acid 2-methylbenzylamide,
2-(1H-indazol-3-yl)-1H-benzimidazole-5-carboxylic acid
(6-chloropyridin-3-ylmethyl)amide,
2-(1H-indazol-3-yl)-1H-benzimidazole-5-carboxylic acid
(2,3-dihydrobenzofuran-5-ylmethyl)amide,
2-(1H-indazol-3-yl)-1H-benzimidazole-5-carboxylic acid 2-(methylsulphanyl)benzylamide,
2-(1H-indazol-3-yl)-1H-benzimidazole-5-carboxylic acid
(benzo[b]thiophen-3-ylmethyl)amide,
2-(1H-indazol-3-yl)-1H-benzimidazole-5-carboxylic acid 3-methylbenzylamide,
2-(1H-indazol-3-yl)-1H-benzimidazole-5-carboxylic acid 3-chlorobenzylamide, and
2-(1H-indazol-3-yl)-3H-benzimidazole-4-carboxylic acid 2-(methylsulphanyl)benzylamide.

5) The compound according to claim 2 wherein R1 is pyrazol-4-yl.

6) A process for the preparation of a compound of formula I 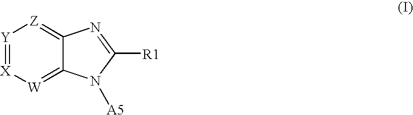
wherein
X is C—R2;
Y is C—R2 or C—R3
W and Z are each C—R3;
R1 is aryl or heteroaryl wherein heteroaryl is selected from the group consisting of pyrazol-4-yl, triazolyl, imidazolyl, indolyl, indazolyl, and thienopyrazolyl, wherein said aryl or heteroaryl is optionally substituted with one or more X1, X2 or X3 selected from the group consisting of H, halogen, haloalkyl, OH, R4, NO2, CN, S(O)nR4, OR4, NY1Y2, COR4, —C(═O)NY1Y2, —C(═O)OR4, —C(═O)OH, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2, —OS(O)nR4, —OC(═O)R4 and optionally substituted thienyl; or
R1 is a saturated 5- or 6-membered monocyclic heterocyclic radical or a bicyclic heterocyclic radical having no more than 10 members wherein said monocyclic or bicyclic radicals contain at least two heteroatoms which are nitrogen and optionally contain other heteroatoms selected from the group consisting of O, N and S and wherein said monocyclic or bicyclic radicals are optionally substituted with one or more X1, X2 or X3 as defined above;
R2 and R3 may be identical or different and are selected independently of each other from the group consisting of H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2 and —OC(═O)R4, or
R2 is H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, N(R6)C(═O)OR4, —S(O)nOR4, —S(O)nNY1Y2, —OC(═O)NY1Y2 and —OC(═O)R4 and R3 is alkyl, haloalkyl, halogen or OR6, or
R2 and R3 together form a 5- to 6-membered ring containing one or more hetero atoms, which may be identical or different and selected from the group consisting of O, N and S;
R4 is alkyl, alkenyl, alkynyl, cycloalkyl, aryl, heteroaryl, cycloalkylalkyl, heterocycloalkyl, heteroarylalkyl or arylalkyl, wherein said alkyl, alkenyl, alkynyl, cycloalkyl, aryl, heteroaryl, cycloalkylalkyl, heterocycloalkyl, heteroarylalkyl or arylalkyl may be optionally substituted with one or more halogen, alkyl, hydroxyalkyl, OH, OR5, C(═O)NY3Y4, NY3Y4, alk-NY3Y4, C(═O)OR6 or optionally substituted aryl;
R5 and R6 may be identical or different and are each independently selected from the group consisting of alkyl, alkenyl, cycloalkyl, heterocycloalkyl, aryl, heteroaryl, arylalkyl, cycloalkylalkyl, heteroarylalkyl and heterocycloalkylalkyl;
Y1 and Y2 may be identical or different and are each independently selected from the group consisting of H and optionally substituted alkyl, alkenyl, cycloalkyl, heterocycloalkyl, heterocycloalkylalkyl, aryl, arylalkyl, heteroaryl, heteroarylalkyl, alkoxyalkyl and aryloxyalkyl; or
Y1 and Y2 together with the nitrogen atom to which they are attached form a ring;
Y3 and Y4 may be identical or different and are selected independently of each other from the group consisting of hydrogen, alkenyl, alkyl, aryl, arylalkyl, cycloalkyl, heteroaryl and heteroarylalkyl, or
Y3 and Y4 together with the nitrogen atom to which they are attached form an optionally substituted ring;
A5 is H or alkyl;
n is an integer from 0, 1 or 2;
wherein said alkyl, alk, alkenyl, cycloalkyl, heterocycloalkyl, aryl, arylalkyl, heteroaryl and heteroarylalkyl as defined above may be further optionally substituted with one or more halogen, hydroxyl, cyano, alkyl, alkoxy, acylamino (NH—COalk), —C(═O)OR6, acyl —C(═O)R6, hydroxyalkyl, carboxyalkyl, S(O)n-alk, S(O)n—NH2, S(O)n—NH(alk), S(O)n—N(alk)2, CF3, OCF3, NO2, arylalkoxy, aryl, heteroaryl, aryloxy, aryloxyalkyl, —C(═O)—NY3Y4 or NY3Y4, wherein the latter radicals containing alkyl, alkoxy, aryl or heteroaryl may be optionally substituted with one or more halogen, alkyl, acylamino or free, salified or esterified carboxyl; and
wherein said phenyl as defined above may be further optionally substituted by dioxole,
wherein when R1 is indazol-3-yl and the compound of formula (I) is the compound of formula (F) 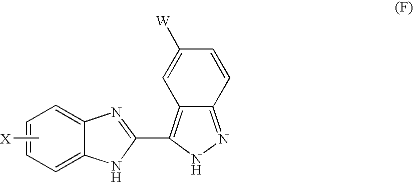
and X is H, R2 or R3 as defined above, then W is H or unsubstituted alkyl;
comprising:
protecting each reactive function of a R1—CO2H wherein R1 is as defined above with a suitable protecting group to provide a carboxylic acid of formula (D)
R1′—CO₂H (D)
wherein R1′ is a protected R1;
esterifying the carboxylic acid of formula (D) to provide a carboxylic acid ester of formula (II)
R1′-CO₂alkyl (II);
reducing said carboxylic acid ester of formula (II) with a suitable reducing agent to provide an alcohol of formula (III)
R1′-CH₂OH (III);
oxidizing said alcohol of formula (III) with a suitable oxidizing agent to provide an aldehyde of formula (IV)
R1′—C(═O)H (IV); and
condensing said aldehyde of formula (IV) with a diamine of formula (V) 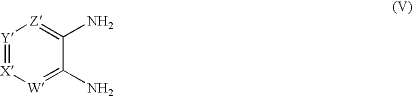
wherein W′, X′, Y′ and Z′ each has the meaning as defined above for W, X, Y and Z and wherein each W, X, Y and Z reactive function is optionally protected with a suitable protecting group to provide a compound of formula (I′) 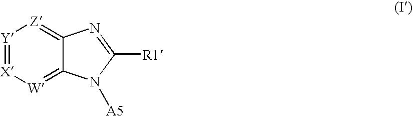
wherein R1′, W′, X′, Y′ and Z′ each has the meaning as defined above and A5 is H; or
condensing the carboxylic acid of formula (D) with a diamine of formula (V) 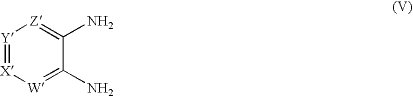
wherein R1, W′, X′, Y′ and Z′ each has the meaning as defined above for R1, W, X, Y and Z and wherein each said R1, W, X, Y and Z reactive function is optionally protected with a suitable protecting group to provide a compound of formula (I′) 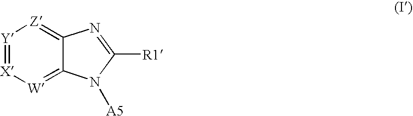
wherein R1′, W′, X′, Y′ and Z′ each has the meaning as defined above and A5 is H; and
deprotecting said compound of formula (I′) to provide a compound of formula (I) 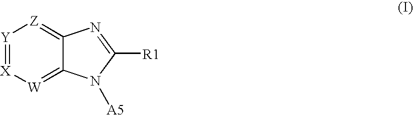
wherein R1, W, X, Y and Z each has the meaning as defined above and A5 is H.

7) A method for treating or preventing a disease or disorder by inhibiting a kinase protein comprising administering to a patient in need thereof a therapeutically effective amount of a compound of formula (I) according to claim 1.

8) The method of claim 7 wherein the kinase protein is selected from the group consisting of tyrosine protein kinase, FGFR1, FGFR2, FGFR3, FGFR4, FGFR5, flt-1, IGF-1R, KDR, PDGFR, tie2 and VEGFR.

9) The method according to claim 8 wherein the kinase protein is tyrosine protein kinase.

10) The method according to claim 8 wherein the kinase protein is KDR.

11) The method according to claim 8 wherein the kinase protein is tie2.

12) The method according to claim 7 wherein the disease or disorder is selected from the group consisting of a disorder of proliferation of blood vessels, uncontrolled angiogenesis, a fibrotic disorder, a disorder of proliferation of “mesangial” cells, a metabolic disorder, an allergy, asthma, thrombosis, a disease of the nervous system, retinopathy, psoriasis, rheumatoid arthritis, diabetes, muscle degeneration, a tumor and a cancer.

13) The method according to claim 12 wherein the disease or disorder is uncontrolled angiogenesis.

14) The method according to claim 12 wherein the disease or disorder is a tumor.

15) The method according to claim 14 wherein the tumor is a solid tumor.

16) The method according to claim 12 wherein the disease or disorder is a cancer.

17) The method according to claim 16 wherein the cancer is selected from the group consisting of breast cancer, stomach cancer, cancer of the ovaries, cancer of the colon, lung cancer, brain cancer, cancer of the larynx, cancer of the lymphatic system, cancer of the genito-urinary tract including cancer of the bladder and cancer of the prostate, bone cancer and cancer of the pancreas.

18) A pharmaceutical composition comprising a pharmaceutical carrier and a compound of formula (I), or a stereoisomer, a racemate, an enantiomer or a diastereoisomer of said compound or a pharmaceutically acceptable salt thereof, 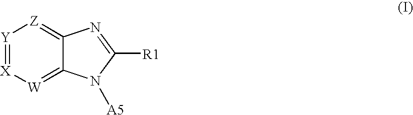
wherein
X is C—R2;
Y is C—R2 or C—R3
W and Z are each C—R3;
R1 is aryl or heteroaryl wherein heteroaryl is selected from the group consisting of pyrazol-4-yl, triazolyl, imidazolyl, indolyl, indazolyl, and thienopyrazolyl, wherein said aryl or heteroaryl is optionally substituted with one or more X1, X2 or X3 selected from the group consisting of H, halogen, haloalkyl, OH, R4, NO2, CN, S(O)nR4, OR4, NY1Y2, COR4, —C(═O)NY1Y2, —C(═O)OR4, —C(═O)OH, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2, —OS(O)nR4, —OC(═O)R4 and optionally substituted thienyl; or
R1 is a saturated 5- or 6-membered monocyclic heterocyclic radical or a bicyclic heterocyclic radical having no more than 10 members wherein said monocyclic or bicyclic radicals contain at least two heteroatoms which are nitrogen and optionally contain other heteroatoms selected from the group consisting of O, N and S and wherein said monocyclic or bicyclic radicals are optionally substituted with one or more X1, X2 or X3 as defined above;
R2 and R3 may be identical or different and are selected independently of each other from the group consisting of H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, —N(R6)C(═O)OR4, —S(O)nOR4, —S(O)nNY1Y2, —OC(═O)NY1Y2 and —OC(═O)R4, or
R2 is H, R4, halogen, haloalkyl, OH, NO2, CN, OR4, COR4, S(O)nR4, —C(═O)NY1Y2, —C(═O)OR4, —C(═O)OH, —NY1Y2, —N(R6)C(═O)R4, —N(R6)SO2R4, —N(R6)C(═O)NY1Y2, N(R6)C(═O)OR4, —S(O)nOR4, —S(O)nNY1Y2, —OC(═O)NY1Y2 and —OC(═O)R4 and R3 is alkyl, haloalkyl, halogen or OR6, or
R2 and R3 together form a 5- to 6-membered ring containing one or more hetero atoms, which may be identical or different and selected from the group consisting of O, N and S;
R4 is alkyl, alkenyl, alkynyl, cycloalkyl, aryl, heteroaryl, cycloalkylalkyl, heterocycloalkyl, heteroarylalkyl or arylalkyl, wherein said alkyl, alkenyl, alkynyl, cycloalkyl, aryl, heteroaryl, cycloalkylalkyl, heterocycloalkyl, heteroarylalkyl or arylalkyl may be optionally substituted with one or more halogen, alkyl, hydroxyalkyl, OH, OR5, C(═O)NY3Y4, NY3Y4, alk-NY3Y4, C(═O)OR6 or optionally substituted aryl;
R5 and R6 may be identical or different and are each selected from the group consisting of alkyl, alkenyl, cycloalkyl, heterocycloalkyl, aryl, heteroaryl, arylalkyl, cycloalkylalkyl, heteroarylalkyl and heterocycloalkylalkyl;
Y1 and Y2 may be identical or different and are each selected from the group consisting of H and optionally substituted alkyl, alkenyl, cycloalkyl, heterocycloalkyl, heterocycloalkylalkyl, aryl, arylalkyl, heteroaryl, heteroarylalkyl, alkoxyalkyl and aryloxyalkyl; or
Y1 and Y2 together with the nitrogen atom to which they are attached form a ring;
Y3 and Y4 may be identical or different and are selected from the group consisting of hydrogen, alkenyl, alkyl, aryl, arylalkyl, cycloalkyl, heteroaryl and heteroarylalkyl, or
Y3 and Y4 together with the nitrogen atom to which they are attached form an optionally substituted ring;
A5 is H or alkyl;
n is an integer from 0, 1 or 2;
wherein said alkyl, alk, alkenyl, cycloalkyl, heterocycloalkyl, aryl, arylalkyl, heteroaryl and heteroarylalkyl as defined above may be further optionally substituted with one or more halogen, hydroxyl, cyano, alkyl, alkoxy, acylamino (NH—COalk), —C(═O)OR6, acyl —C(═O)R6, hydroxyalkyl, carboxyalkyl, S(O)n-alk, S(O)n—NH2, S(O)n—NH(alk), S(O)n—N(alk)2, CF3, OCF3, NO2, arylalkoxy, aryl, heteroaryl, aryloxy, aryloxyalkyl, —C(═O)—NY3Y4 or NY3Y4, wherein the latter radicals containing alkyl, alkoxy, aryl or heteroaryl may be optionally substituted with one or more halogen, alkyl, acylamino or free, salified or esterified carboxyl; and
wherein said phenyl as defined above may be further optionally substituted by dioxole,
wherein when R1 is indazol-3-yl and the compound of formula (I) is the compound of formula (F) 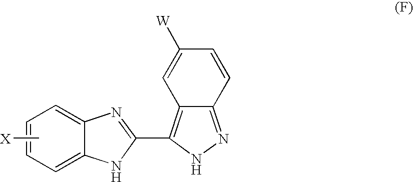
and X is H, R2 or R3 as defined above, then W is H or unsubstituted alkyl.

19) The pharmaceutical composition of claim 18 further comprising one or more antimitotic compounds.

20) The pharmaceutical composition of claim 19 wherein said one or more antimitotic compounds is selected from the group consisting of taxol, cis-platin and DNA-intercalating agents.
